SEQUENCE LISTING (101 sequences)

SEQ ID NO: 1 - DNA, 38881 bp, synthetic construct
  misc_feature 1..38881: note = pSIM4363 vector
  source 1..38881: mol_type = other DNA
tggcaggatatataccggtgtaaacgaagtgtgtgtggttgatccaaaatctatcgtacctttagaaagtgtagctatgaaggatagtctcacttatgaagaactacctattgagattcttgatcgtcaggtccgaaggttgagaaaaatagaagtcgcttcagttacggctttgtggaggagtaagggtaccacattgattgagttttatatgcaatatagtaataataataatatttcttataaagcaagaggtcaatttttttttattataccaacgtcactaaattatatttgataatgtaaaacaattcaattttacttaaatatcatgaaataaactatttttataaccaaattactaaatttttccaataaaaaaaagtcattaagaagacataaaataaatttgagtaaaaagagtgaagtcgactgacttttttttttatcataagaaaataaattattaactttaacctaataaaacactaatataatttcatggaatctaatacttacctcttagaaataagaaaaagtgtttctaatagaccctcaatttacattaaatattttcaatcaaatttaaataacaaatatcaatatgaggtcaataacaatatcaaaataatatgaaaaaagagcaatacataatataagaaagaagatttaagtgcgattatcaaggtagtattatatcctaatttgctaatatttaaactcttatatttaaggtcatgttcatgataaacttgaaatgcgctatattagagcatatattaaaataaaaaaatacctaaaataaaattaagttatttttagtatatatttttttacatgacctacatttttctgggtttttctaaaggagcgtgtaagtgtcgacctcattctcctaattttccccaccacataaaaattaaaaaggaaaggtagcttttgcgtgttgttttggtacactacacctcattattacacgtgtcctcatataattggttaaccctatgaggcggtttcgtctagagtcggccatgccatctataaaatgaagctttctgcacctcatttttttcatcttctatctgatttctattataatttctctcaattgccttcaaatttctctttaaggttagaaatcttctctatttttggtttttgtctgtttagattctcgaattagctaatcaggtgctgttatagcccttaattttgagttttttttcggttgttttgatggaaaaggcctaaaatttgagtttttttacgttggtttgatggaaaaggcctacaattggagttttccccgttgttttgatgaaaaagcccctagtttgagattttttttctgtcgattcgattctaaaggtttaaaattagagtttttacatttgtttgatgaaaaaggccttaaatttgagtttttccggttgatttgatgaaaaagccctagaatttgtgttttttcgtcggtttgattctgaaggcctaaaatttgagtttttccggctgttttgatgaaaaagccctaaatttgagtttctccggctgttttgatgaaaaagccctaaatttgagttttttccccgtgttttagattgtttggttttaattctcgaatcagctaatcagggagtgtgaaaagccctaaaatttgagtttttttcgttgttctgattgttgtttttatgaatttgcagatgcagatctttgtgaaaactctcaccggaaagactatcaccctagaggtggaaagttctgatacaatcgacaacgttaaggctaagatccaggataaggaaggaattcccccggatcagcaaaggcttatcttcgccggaaagcagttggaggacggacgtactctagctgattacaacatccagaaggagtctaccctccatttggtgctccgtctacgtggaggtggatccatggcggctgctgcctcaccatctccatgtttctccaaaaccctacctccatcttcctccaaatcttccaccattcttcctagatctaccttccctttccacaatcaccctcaaaaagcctcaccccttcatctcacccacacccatcatcatcgtcgtggtttcgccgtttccaatgtcgtcatatccactaccacccataacgacgtttctgaacctgaaacattcgtttcccgtttcgcccctgacgaacccagaaagggttgtgatgttcttgtggaggcacttgaaagggagggggttacggatgtatttgcgtacccaggaggtgcttctatggagattcatcaggctttgacacgttcgaatattattcgtaatgtgctgccacgtcatgagcaaggtggtgtgtttgctgcagagggttacgcacgggcgactgggttccctggtgtttgcattgctacctctggtccgggagctacgaatcttgttagtggtcttgcggatgctttgttggatagtattccgattgttgctattacgggtcaagtgccgaggaggatgattggtactgatgcgtttcaggaaacgcctattgttgaggtaacgagatctattacgaagcataattatcttgttatggatgtagaggatattcctagggttgttcgtgaagcgttttttctagcgaaatcgggacggcctgggccggttttgattgatgtacctaaggatattcagcaacaattggtgatacctaattgggatcagccaatgaggttgcctggttacatgtctagattacctaaattgcctaatgagatgcttttggaacaaattattaggctgatttcggagtcgaagaagcctgttttgtatgtgggtggtgggtgtttgcaatcaagtgaggagctgagacgatttgtggagcttacgggtattcctgtggcgagtactttgatgggtcttggagcttttccaactggggatgagctttcccttcaaatgttgggtatgcatgggactgtgtatgctaattatgctgtggatggtagtgatttgttgcttgcatttggggtgaggtttgatgatcgagttactggtaaattggaagcttttgctagccgagcgaaaattgtccacattgatattgattcggctgagattggaaagaacaagcaacctcatgtttccatttgtgcagatatcaagttggcattacagggtttgaattccatattggagggtaaagaaggtaagctgaagttggacttttctgcttggagacaggagttaacggaacagaaggtgaagtacccattgagttttaagacttttggtgaagccatccctccacaatatgctattcaggttcttgatgagttaactaacggaaatgccattattagtactggtgtggggcaacaccagatgtgggctgcccaatactataagtacaaaaagccacaccaatggttgacatctggtggattaggagcaatgggatttggtttgcctgctgcaataggtgcggctgttggaagaccgggtgagattgtggttgacattgatggtgacgggagttttatcatgaatgtgcaggagttagcaacaattaaggtggagaatctcccagttaagattatgttgctgaataatcaacacttgggaatggtggttcaactggaggatcgattctataaggctaacagagcacacacttacttgggtgatcctgctaatgaggaagagatcttccctaatatgttgaaattcgcagaggcttgtggcgtacctgctgcaagagtgtcacacagggatgatcttagagctgccattcaaaagatgttagacactcctgggccatacttgttggatgtgattgtacctcatcaggagcacgttctacctatgattcccattggcggtgctttcaaagatgtgatcacagagggtgatgggagacgttcatattgaactagtttttaatgtttagcaaatgtcctatcagttttctctttttgtcgaacggtaatttagagttttttttgctatatggattttcgtttttgatgtatgtgacaaccctcgggattgttgatttatttcaaaactaagagtttttgcttattgttctcgtctattttggatatcaatcttagttttatatcttttctagttctctacgtgttaaatgttcaacacactagcaatttggctgcagcgtatggattatggaactatcaagtctgtgggatcgataaatatgcttctcaggaatttgagattttacagtctttatgctcattgggttgagtataatatagtaaaaaaataggagctcgattctttgaaaagaggcttcatactccctccgttcatcttcacttgttcattatattatttaagaatatttaataatatttatccaatttataaaatcaatgaataatttatcattttattcttgttattaaatattatttattttccatattcagaattatggagtattaaatgaaatatttagttcatgaattaactagtacaaatttttttaaaatcaccttatcgagcatattaaatgtaaataattaacgcagcaaactaacctatgagattttcatattctcagctttgcaagccgtttggaaagacttattggggaacgtagtcttacaagtaacagcaatagtgccgagatcaccaaatgcattgtgctcagtgtgagaggttgcacgaagcttcttagtcaagcatttctcatttgtatatcttttacgattctatctattaatgatttttgtatacatttgattggcatatttctatattttccaacccataggtatttgtataatattttatatttatgcaaatcatcatataagcaagaaattcgaatcaaagagcaaatatatcaaaaatttcaaaaagataccatacatgcaccacaaataaggtctacttttttctattgaaaataagatttttattttattactccattaattaaagataaataaacataaaataaaacaaagagaaatataaaagtaatttgtttttacaaatcacaagcaattaacttgaaacagtaatcaaactttttctcttattattagtattataataccaacaggcatcagaagattctctccaacacagaatcattttcaaaggcatccctgcaatttattagacatggaatatttagttgatgaactaaatacaaatttaaaaaaatcatggatattaaacgtaaataattatttatagtacctgtgatattctcaactttgcaagccgttccgagaccctgagactaattggggaaatgtttgggccttgtacaagaaatagctttttcaatttaggcatactcatatcatccatgcacgttacttcagacaacccttgtgcatcctcaatatacaaaacttcaagttgcggaaagctatcaccagagagatccattttttctgcatcatgtctgcaccaaagcaatataagtatccttaatttgggcaatttcttaagcactgccaatagatgaccgtctaccatcaagccgacaagattaagcttgacaagatttggagggaatgcttcaatgttcaagagcattgctgagaaaccaaccaaacccaactcgacaatatttggatgggacgacaagtttatttgctcactcgtgttcttgaaaaatctgagcttcagaacctccaacgctctaggcacagggctcaatgctgataatatcttaatggtagaaccggatacatccattatacacaatgttcttaaatttatcaatcggtgcaacaatcttggttcacaaaatttatcatccacccacatcaaagtttgtagattattaggaggcatgattcggcaaaatggacttgcaaaagagacacagtaacattcttctgtgtaacaaagatgtctcaatattttagactcccaaacaccaaaaggaagtttactagagctatgaaatatatccagggtctctagacatttgagcttgacaatactatttggcaattttactctaattgccccctccaatcgcacataacgtaggcaactcatgttcccaatttttttggggaaagtaacatgctgataacccttttgagacatgactacaaccaacacttgcaataatttgaagttaagatgagccatttgagaaaatatgcaacggtcttttgtgaaacagaaaagtgaacgaagcttcataggcttaggatttaaatggaagaactcattcatggcattatcatcagagtagaatgtaatccttcgcaccctagcaacattgctaggatcaccaaatgcattgtgctccgtgtgaaagaagttactttccttagccaagtccacacacaaactatgtaacaagtcatgtatgcgacaacttgaaattcttccatcatatgtccttttggcaacttgaatcaagtttcttgaaaccaaatcatttaggacatcatccgccaaactttcagcctctcgcccattgccagtatttacaactatcagcttctcagcaatccacatatttgtcaaatcaaaagcacgaatttcatggtcctcggggtaaagaccaaagtacaagaaacatggccttaatgcaatgggcaaatcattgtaactcagagccaataccttaccacatccatcttgaattttatgagccatactctcaagtactctgttccatgcatgttctgttcttcctcttgcccttaacatgcctgcagtcaccacaattgctagcggtatacctccacatctctcaactatacatctaccaatatttaccaagtctggtgaagcattggcccaattatcattaacaaaattaaagattttcttggtaaagagttcaaagcttttctctgaatctaggggttgcaacacgtggattgagaaatcccctcctatgtatctgcctacattactatttcgagaggttataattatcctactgccaatttttgaatcacattcaggaaggacaagttttagatcatcccaaatttcaacatcccaaatgtcatctaagagaataacatacctttttattttcaagagtgatcgtaggttgttctccaagttttctttcctttcctcttccgtcagtccaacttgtttggctatgtcatgtaagatttctcccgcccttggctgttgtgaaacatagaccagtcccgaacactcaaattgatgacagacatgcctataaagtttcttggcaagagttgtttttcccaaaccgggcatgccaactattgaaacaactccataaggcaaatcatgatcaagtaatttggcttgtagtgtattgaagtcatcttccagaccgatgacctctgtttcatcagcatgaaggaacaatcttctccggtccaatggaatgcaatcatcattattgttacttgtatctgtgatgctgtaagttgtccttacacggtcaatatcagcaactcttctttttatcttctcaatctccatagcaaactcatcggcaaaagaaaccgtcttaaggcaacaaatgaacttattggattgttgaatttttggaagaaactcatctaatagatcctccacatcacctgccagttgttgaatatcttttaataagtttttcacccttgaatcgcctccaacttcctttgcctttgcattgtctacatatgatcgaatgtgtctcatttctctctggagccaatcgatgtcctctttcaaccaatataaacgcgtaccttcttgaaagagtacatttccagctatttctattgatttattgatgactgctgtgagaagaatttcagccatctcctttaatttttcgttaaatttcttttttctaaatgtgaggatgaggaagggaaagtaagagtcaacggcccaagtcttgtaaacacaataattcatcttttgttagctggtatttcgttttcaagcatgaataacaaacgttgaaagtcagaaaattcaggcagtactacgcgtaaaacgaaattagaaagaagaagataattggtaaaggaaaagaaaggtcagcttagaatagttggagttcagggccaagtctcctaataatgtcaagtaaatgaaaaacgaaagaagaaaggaacaacacgtgctgtggatagatactagtagaatagtcaaaagaaattacaccgttcgtcccaatttaagtgtcattcaattaaattcgacatggaatttaaaaaacaaacagtaacttttaatattttgtgactgatgattgataatgtaccttttttttttgaaaaaattacgtaactaagcaaacatctttagtatataactatcaatcacaccatatgttaaaaaaatttattatatatctagcgccattttatgtttcctaactgtttgatgaacgtcttgcaaaactttacccaatttgggttcatggctgtgagtttgggctattatgagactcaatttgtatgttccatgaaaattaggcttttatgcaacttgattctctcaaaatcttttgccatactcatacagagtgggcgttcggttcgatatttatttaactttgatttgatttggtcgtgagacatcatcataatgactcgagtagaatgtagggtgtataactgggcccgtccatatgtggaagctactctctttgtccaaaaatagttgtccactattgacttacacacaccttaagaaataataaataataggggtactattactacactatcctttgaatattataaatttaatgctctgggaaaatgtattagatattgaatactccctccgtttcaaaaagaatagtctactttgacttggcaaagagttttaaaaaataaagaaaatttttcaatcttgtagttctaaattaatgttatgtcaaatgtaccaaaatatcttttaatcttgtagccttgaacatgccacgtggaaagttgaaattaaagtgttgatcaaaaaggaaagaggtcactattttctaaacggaggggtggtatttaattgtaggggtaaaatagacacaaaaggataaattatcttttgattttctaaattggacaagtattgttggacaactatttttcgtatagtgggcaagtaaagatggacgggggaggtattaccaagtctaaaaatgggaagctatggccaagtctaaactgatactccatgtgttcctttttatttgtcacgtttttttttttttgagaaatcaaactacatgaattttgaccaatattttaagatctactttttcaaatagttctcaaacatctaaattttaaataataaattattctaattcaaattagcttcaaaaattagtcaaattgactttcagaaagtcaattgtgacgattaaaaaagaatggagagagtaagtttttttaaaatcttattctccattatttacatgacgagtcttggagaattttcactttgttaaggattcgattcttcatatcgtaatcccttcccctattcttcctttcacctgcccctttttgaaaattaaatatgacccagaggctgcttttttatatattgcttcacataccccatttttttattttttattttgaaaaacggaaaaagacatgacgagccccactagtctttcattatttctcattttttgtacacattgaatttctttggaatttaataaaggtagtacttagatttgaccaatccaaactatgtgcctttggcgtgttgctttttttaaaaaaattattttttgaaacttttgataataagttgtcacatgtgtagcatttttgactttgatcttcaaggcttattgagggcaagtggggtcgaaaattcaatatcaccgatttataattggatggggatttttatttacatctggcattagtcgattttgaaaagaatgtctctttcgtaatttgagaactcgttaatcacaatattgcacatcaacagataacatgtttaagatcacaaaattaaaaaacaatttgatatattattacacatatctttagtttaacactataagattcaaaaatcttctttggtttctgaaacttcattgtcgaatcaaataaaaacgaaaaagttgaaatagagagagaaaaggtagtttccttgaaatttctctagatttggagcaatccaatgtttttctgtatcaaaattttacatattgatggtgagtactgtcaataatgtagtactccctctgtcccaatttatatgactcactttcctttttggtcagtcccaaaaagaatgacacatttctacatttagtaacaatttatttttaaaatgacttttttactcttaatgaaatgatttctagccacacaaatatctaccactcattttagaccacaaatttcaaaagtcttcctttctttgttaaactccgtgccgagtcaaactatgtcatataaattgggacggagggagtatgatatttcttctcttttgagtaatatttaatatcttcaattaaatcctatccactcctcatctattcttgttcatcatttttgagaattaatttgatggcagcttatagtgctgtaatttctcttcttcaaacacttattgatcaacaaaatatttcagaactctttcatggtcacactgcccaaacgctcgattctcttcacactacagctgaatattttcaacatgtccttgaaaatattacaagatttgactctgaaaagatcaaatctttggaggaaaaaataagagttgttgttagctatgcagaagatgttgttgcaatgaaaatttctcaaatcatcataggctcaagctggacatttggaattttacaacaccaggatttactaccacttgttgaaaaaatggacacaacaaagaaacaagtgatggacattctttctcatgatgatgatcaaattcttgaattaactgcaggggattccttgattggcacttcttctacaacttatccaatgttggaagatgatatcgtgcagggaattgatgatgacttggagatcatagttaaaagattgacaggaccaccacgggatttagacgttgtcacaataacaggtatgggtggcattggtaaaacaacactcgctagaaaggcttatgatcatctcacaatcaggtatcactttgacattcttgtttggattacaatatctcaagaatttcgatgtagaaatgtattgttagaagctttacattgcatttcaaagtcaacagatattgtgaacacaaaagattatgataagaaggatgacaatgagttagctgacatagtacagaaaaaactaaagggtccaagatacctagttgttgttgatgatatttggagtagagatgtttgggatagtataagaggaatatttcctaattacaacaacgggagtcgaatcttattgactactagggaaaacgaggtagcaatgtatgcaaatacttgtagccctcatgagatgagtcttttgagtttagaaaacggttggaggttactttgtgataaggtgtttggaccaaaacatgatcatcctcctgaattggaagaaattggtaaggaaatagttgaaaaatgccaaggactacccttaacaatttcagtgattgcgggacatgtttctaaaatgcccaggacattagaatgttggaaggatgtcgcccgaaccttaagtgaaatcatctctagtcatccagataattgcttaggagtgctcggtttgagttaccatcacttgcctaatcacctcaaaccttgctttctttctatgagtagtttcccagaagattttcaggttgaaactcggagattgatctacttatggatcgcagaaggtttcataaggacttgcgaaaatggtaaaagtttggaggaagttgcagtagattatttggaggaccttattagcaggaacttgatacaagctagaaaaaggagattcaatggtgagataaaagcatgtggaatacatgatctactgcgtgagttctgtttgatagaagctgaaataacgaagcatatgcatgttgagagaacttacccaactcttccaacacaaaagaataatgttcgtcgcttcagttttcaaacaaaattttattcagttgatgattgtaataagctattaccacctgttgccagatctatctacttcttttctcagttggatctacctgttgtaccctataagaggtatctcaggtgttgtttgcccatccaccgtgatgatcgtataatacatgatttttactctcgtttcaaccttctcagggtattggtcatctccaagacaaatgaatacttcgagtcatttccacttgtgattacaaagttgtttcatttgagatatctccaagttcgatttcttggagacattcctgaatcaatctcaaaccttcaaaatttgcaaactctaatttgtagtggtggtactttacctgggaagatatggatgatgaagaacttgaggtatataagtataataggcaacaaagtcacttatttaccaagtcctagaacagaaagtcttgtgaatctagaggagttttctgttctttgttacagaagttgtacaaaagaagtcatttctggcattcccaatctaaagagattgaccattgatgtactttctagcattaacaactattttcccaatggactaatagatatgtccagcttgacaaaactcgaagcattcaagtgtaataggtgtttatattcgaatttcaatagttctgttattccaacatcacttaaggattttgtttttccaacatcacttaagaggttgagtttaaactattatgctagtcattttttttgggaagaaatatcatcaactattatcatgttgcctaatcttgaagagctgaagcttaaagattgtcgatccgatgaatatgatgaatggagtttgagtgataaagacaaattcaaaagcttgaagttgttggtactaaccgacattttttttgatcgttgggaagctaccagtgataacttcccaaatctaaaacgccttgttctgaacaagtgcgacttagaaattccatcagattttggggaaatttgtactttggaatcaattgagttacatgattgcagcactagtgctgaggattctgcacgagagattgaacaagaacaagaggagatgggaaataatatccttaaggtctacatacatggcagtcgcagtaagttctaaacagttattcgattctctcatctttgttaaatcttacctccttgaagttatgatgatgacttcgattttttgtcttgcataggcaggaaaatggaagcatatttcgaggaatcatctaatgccatcttccaaatgcagaaccttttagattaatcttttatgttctgattgtttgttaaattcatgttttaggaataaacacgtttaatacatgtttcgctctttctttactatgtaaacagtaactattgatgtattgttatatctattttgtgttatgtgatagttcgaaatgaataatgtgttggggttttggtctttttcccttcctatgtggataaataaaagcccaattggtctagcccacttatgcccataagcccaatttatggagcatacataagactttctttaagtcttattttcatatatcacacataatttttacaagagagataagcatacatattagagagaaaagagagaaagtgcagattttcgcacaaaccctagcagccaatttcaaatcgcgattcccgcttcgtttcttatctgattaagctgatttttggacagcacgttcctctcaacttaatctttgattgagaactgacagaggtcgatttggagtcccgtagctccagatttttgctcgtgaacagcagctgctatttggtgattttgctccttttacttctctagttctttggtgctatcttgtagttgttgttgctcgtttttggcacttgttttggtgaccattttggagaggatcccccggggatctagaatttaagctttatggatgtaatattatttttaaagttatatgttcacatctattatttgttataattttcatgaaaatttatatatatatattttgcatgtatgttgcagtgaatatcttagattagtgtattttattatcagggtttttgtaatatttcatttttttatgtgggcacctaacatagctatagagagctttctttcaagttctcacgatcggacgtataaaaagtacctacataaaactttactcaggagaaagaagaggaaagcagtagaaaaagaatacaagaaactgaagatcaaaagcagcataaaaagtcaaaacctctcactacaaatatctttcctatttttatgcctcatatattattttatctaccctccccatcatgtttcattccttcaaaccatattatataggaaactcaattttgtaaatatgtacgtctacctggctaatattcatattacaagttgactaagacttgtggagattgtcagcttgtgaatgaagatgatgtctgatgcaactttggtgttgtggtttatgagaattgacgggagcctacgagcaaccataaagttgtctccatgtaacctatagatcatgggtttgagcggtgaaagaagccactaatttttctacagtatcctcctgggatgcgatccctccattaatcctaccaacgttggatgttttgtgcatgaggctgctttgtttacgggacattcctgatgtttcttgtttttcatagttcccctttagataaagtaaacaagagagtttgaagatgaacttaccttttgacagtctttagtttagcaatattagaagcccttgtttgagaaataatggcaaagagagttgtttgctaaatgaagtatagttgaatgataaattttatatacctagaaacaaggatcaaattcccacttcatacttccttttttctcctttcattagtggtgcattcatgctctagaaattctagattcgcttctgaggtgtaaatttgtattagcagtaaaaatctcaaagaggaatgtacaatctcttagagtcgagatacacctacacatcttgcttttactgttctatcgggagttacttcatgtattgcttgacataaaactgattgtggattcgtcttggtagatatccaggtaatacatcctgtctgctctagccacactaaccatatgcatcaggcttgaaatcgccctccgggatagtatgtgcttgtcaacttagagttggaaccaccatctgctgactgctcaaacttcacctcattaagatatttttcgagtttgtcatcaatcctatactttatgaatttaaagttgccacctgcagaaaagaagtcacaaatagaagaatattagtgattaaaatgagagtctattaaaaattgaaaaagaattaactaggatgaagaatgttgatattcatgacaaattcaatacttctgtagcttgtggctctaacacatttcagattctcaaaagtgtcttcctcccccatgttccattcttctccctgaatgattgtataataaaggattcctctccaacctccagtcttgctattgttgatagtgcatcggatgtcagaggaaagtcatacaacaacaattttttcaaattcgagattagagactgcccctttggcgatgaggctctgttggctaatgctgcaaagctggagacaatgcgatccctttggatgtctaattgttcagtaagttttgaagcatgtaagctgttggtccagaagttgccagggcttaatgttgaagttatagacgagaggggtcatccagatacgagaccagaaagttgccctgttgagaaactttacatatacaggacagtgtcaggaaggaggttcgacactcggattattgatgaagatgcagcattgacttgttcttctgttgaaatggtgctgtcaatgtacttgtggtcatgaattgtcgtaattctttttgaagattaatgtgaacctttcattcactttgagtgctggaaatcccatttgttgaacgtttagaagaaggtatttcagtgttctactacgagagttaatttgataaacaaacacttattttcgttcagagtgggatttcagcctacgttggttgaatgttcgagaaaaggaagtcatgagtgcttataagctctctacaacctcttcacttcctggcctagcttttgagatgaaagttcgttgggtttgtgataaagtagtatagatgtcattttaaatcagtaccttgattgaggttgaacgatagtgctctgagcatatgagacttcagaccaagttggacagccaaaggcaaatccacattgtatttataaaaactataatttttgttaaaaacttctgagttttctaaaagaaactcagaaaagtcaaataaaaatgtttcataacaactgtaacagaattttcttaaccatatacaatctacccagcaaagtgcaatctacttttcaaccataatgctcaaaaactacttaaataacgggatatccttctggccaaggatctgaagctcgtcccctcccctcatatcttcagcatattccttaatcttgagagcggaattttcaagttgagggctccttacaagttcgataattttcaaggaataaatatccccaaaactagacggaatctcctcaagattatgacagtccgacagttctaatttctcaagcgtgggaaaagattcctctccaacctcccacttggaaagaatcacttgactcaacatcaaacatttgagattctcaaaggtgtcttcttctcccatgttccattcttccccatggatgattgtacgataaaggtacaactcttcaaggttcagcagtctcgctattgttgatagtgaatcggatgtcagaggaaattcatgcaattgcaatcttttcaaactcgaaggaaagtgaaaatcccatggccgatttatggctgcagaggacccactgtcatttgtgtttgatctttcaaaatctacagtgagtttttctagttcagttaggaaatccaatttcgggaaccaatattgctctgttgaataatcccatgactccttgagtttgaaatgaagcacttgaagattgggaagccttttgaaaatatcctctgtatctttccaataggaaagcacgagttcccctaatgctgtcaagttctctaactttgtgtcctctgctatcagtattgattcatctgcatccatatcaaagaaagaacaagcagtcgtgaacagcacttgcaactttacaagatcccaaattctcggtaatagtatcaaggttgattctttgttatccacaaacaagatttctagattccagaggtttgagaaagacaaaggcagagatttaacttctgtcccaatgcttaagtacctcaaatgattcaacatgcatatttcattcagcaaagaatctttaaccatgataaaagaggattccaggtgcaaggttctaagaagcctcaagtgtcttagatgaaatgtatcagaaagatggtcgtccagctcatctccatttatggtcaaagaatagaggtgtttaccggaatgccttttcttatttgaaccgaacaggacaaaattaagcccaaagtgctcttcatcatcatcataatcaatgctaatttgacgtggcaacaaatctgatggagcacttgaacttatccgatcacacaacttttcctttcttgcttttatcaaacaaaagtcatgcacaagatcatgaagttggcaagtagggtaatcacctatctcattgaaacaaattaccaagctactggaaattaaatcatccaaataaattttcaccacttcttccagactcttcatatctgtcttttccacaaatccttcaaaaccccaaataagtttcaactcatggattgtcgttacccagtccttcggcgcacttgcaaagtacagcaagcatggcttcaggtgatcaggtaagtggtcataacttatttctataactttcatcacttccacttcattcttcaaaataaaggaatgcaaattatttacaacttcaagccacacactctttttcttttccctcccagcaatgattccagcaatcagatccaccaccaaaggaagccctttacaattttcggctatttctttaccaacatccaatagttcatcagggcaactctcgtttccaaatgcccttttctctaataactcccaactttcttctgatcttagcaatcgaaggttaagaggatcagtgtagagctttccatgcaaagcaactttcttttctcgagttgtcaaaataattctacttcctttcataccatcaggaaaaggtcttgttagctcatcccatgtattagtatcccacacgtcatctaagacaataagatacctctttccaaacaattgtttccgtagtttatcagcaacatcaatattctcactcaattttgaatttgagtcactaacttgattgaaaattttatccaacaacttcttctcgtcatatacttggtcgaccgtgcaccatgcacgaaggtcgaaatggctagaaactgatttatcattgtatactttgtacgccaaagtagttttacctaaacccggcataccaatgatcgaaatgacatctagatctgccggtccactggtgagctttctaagtatcaagtttgtctcctcaccaaaacctacaattattttatcagttgtcaatgacttgctctcaactggtttcttgggagagttcacaacgatgagacctctgttcttggaaatgttctcatgtaaatcagagacctcttctttgataagcatcatcttctttctggtaatgggaagtgagaaaataagatgtaagagaccattatctcgaacaataattgaatctatgacatcttttgcctcatatgccacatctagaacacgttcccagagatctttatacaatccttgctcaatattcgcgaaaaaagatcttatgaattccaagtcttctttcaccagcccaatttgttcctttatcaaagcaattgaataagcattggaatccagcaaatcatctaagtgtctctgtagcagatgcatgaagagaggtccatcactcatggggaagcaatattgagatgaatccgggactttcagataaacatgtttgagatcttccttaaggagttcaatattttccagaaactttagggttgcacaatttgtttcgtcagtactctctttaatccttaatttctcttccaagtcgcgtacaagagttgatacctccctggtaagtgctacaacacgagccaagagatcaaaaagtttgtcatgatgaataaagtccttgggcggcatatcagaaagaataatcaataggaattccatcatgacatgaatgtttcgagcccctgaagtgttaggggtaataacagttatcatatgctcttgtagatgaatcagatattctctgagaatgtccggagaggtttccaggagcttcttaatgaagcgtccaatttctgttgaagttgaagctttcaaagttttataacatatgtgcataacctccaattcagttggaacaatcttcaagagtagatgtgctagcttgaagagttgagggtctttatcattctgatcatcctcatctagctcggagagttgagagtcttcatcagcctgatcctcccaaaggaagcgtcctactctctcagccatcagttgaaacagagataagacattctcaaccatctcatgcttaatgcaacaattcactatcaatccatggaaatctcttatgttgccacatacattctgaagaacctcatattgagtcactccaggaaacatcttttcagcacgatgcttggatagatgagagagattcaagaggaggaagcccaattgctcatccatcatggtggcatctgatttagaacgatgatacaagcttatacagtcatccatattaccggcgaggctagtaaggacatatttacacccgacgtctttgccatcatcatccaaaattggttgaagcagattctcaacctcttgtctttttctagtcattatatcttcaaacttctccaaatcggaataagaaagctggacatatgtacaaataaatgtcagcttcaatttcaggctttcaatcagatccacatcaacagccttttgatcttcttcattctttaatctctctaggaaatccagaacattggcagcatccttgcgaagagcagaaaatgactcctgccaaaataacacatatcaaatttttaaagccacatgatatctacaattcaatactttacagttcactctatcaaataacataccaatgagttgtttgcttcttcattatcttttcgtttttccatgattctacttttcctttccaagttctcaagacctgatgcataaaaagtacataaaactttatcaggagaaagaagatgaaatgataagaataatcaataatttcactattttcacgcgaacttaagaggacttaattagtggaagatgataaaggtacacttgcgatttgaattgaagaaaatgacttgtaaaaatggggttcatccagaaacgagaccaactggtaccgaacctttctatctctaatagcgtatagtaatatattttcattagtgaggggaatctttcgtcatcttagaaaaaatctaaaacatctgcttctgttcactccacactctccaaccccatccctctcacccggtaagaattcacccgcaccggatattaataccaaattaatacaacttatcatgggtaagtaataatttaatgaggtgaaagtatacattaactaaccacaattaagtgatttgttgtaaatcggtggtgtgagtaagtatttaacctctctctttctcctgcaatttatttccctttctctttgttagatccaactaacctactaaagttctttcatttttcttacatcacttctacttttccataactgtacttcaatagagatgagagtagcaaatattagatgagttgactaacctttttagttgatgaaactatatatcctctgcttgctttgttattagatgtatagagctcaaaattctggaaataaacagaggaataattaagaccaaatatgatgcatagtattgttccaaaaagtcaagaaagcaaaagcatgaatcaattacagtggggcctcctcacttgtctgccacaaattaggaaaaagataaagaaaatgagaaaatagtcaaatgtcccctaacctatactcaaattttcaattacagacttaaatttcacaagggtctttgttaattaccatcgattttcaagacctccaatgttaattggatgtgtttttatttttaatttattatttgatttattcatatttattcattattaacacttttactgtgtgtctttacaggtacacatgtttgcttctcattgaagctattcgaatgaagttcgaacaacattctttatgttaagcagttagttaaatgcactgcagtgcactaacagaaaatgtacacctatcagtctagttgttagagtcagttaaattaactgcttcagattagaatataaatagagatatatagattgtaatagtatagttcttctcattatttttttcattcattttggttcatgagttcaatatagaatttttctctcttcttctccttcaatcagtctttctaaaatagttaattctctccattgtttagttagaattcactgaaattacatggtatcagagcatttgaagcagatctagggttttatctcgggattttttgagcatagatcgagagaatctgcaatggcgattgaagaggaatcagtggatgaaattcatggtagaactggaggagctgtaccgaggggaggttcggtgattctagatcgcggccgcgaaccatgcatctcaatcttaatactaaaaaatgcaacaaaattctagtggagggaccagtaccagtacattagatattatcttttattactataataatattttaattaacacgagacataggaatgtcaagtggtagcggtaggagggagttggttcagttttttagatactaggagacagaaccggaggggcccattgcaaggcccaagttgaagtccagccgtgaatcaacaaagagagggcccataatactgtcgatgagcatttccctataatacagtgtccacagttgccttccgctaagggatagccacccgctattctcttgacacgtgtcactgaaacctgctacaaataaggcaggcacctcctcattctcacactcactcactcacacagctcaacaagtggtaacttttactcatctcctccaattatttctgatttcatgcatgtttccctacattctattatgaatcgtgttatggtgtataaacgttgtttcatatctcatctcatctattctgattttgattctcttgcctactgaatttgaccctactgtaatcggtgataaatgtgaatgcttcctcttcttcttcttcttctcagaaatcaatttctgttttgtttttgttcatctgtagcttggtagattcccctttttgtagaccacacatcacgactagtcattacacattcctcccggatcaacccgattccggccaccggaagtcccttaaaatcatctccggcattttcctctcctctttccttttgctttctgtagccttctttccgatcctcaacaaccagtcaccggacttgcagagtaactcccgttcgccggcgccgccgtcaagaggtgtttctcagggagtctccgataagacttttcgagatgtcgtcaatgctagtcacgtttcttatgcgtggtccaatgctatgcttagctggcaaagaactgcttaccattttcaacctcaaaaaaattggatgaacgatcctaatggtccattgtaccacaagggatggtatcatcttttttatcaatacaatccagattcagctatttggggaaatatcacatggggccatgccgtatccaaggacttgatccactggctctacttgccttttgccatggttcctgatcaatggtacgatataaacggtgtctggactgggtccgctctcgagataattgtaactgatttattcttgaataacaacttcaatgaaatcaagcaacaaagctgatttcaacataaaaaaacagaacaagaaaacaaaaacagagcatcatccatcaaagtgtaatctcagcagattcaatagagactatctagagtgtatgggtgatccttctcttattataccgactaaagacattggtattaaggatatcttatcttttgaggagattcccgttcagattctggagcgtcaggttcgcaagttgagaaccaatgaggtaacatcagtcaaggtcttatggaggaatcagtagtctctattgaatctgctgagattacactttgatggatgatgctctgtttttgttttcttgttctgtttttttatgttgaaatcagctttgttgcttgatttcattgaagttgttattcaagaataaatcagttacaattattctcgagagcggacccagtccagacaccgttaatatcgtaccattgatcaggaaccatggcaaaaggcaagtagagccagtggatcaagtccttggatacggcatggccccatgtgatatttccccaaatagctgaatctggattgtattgataaaaaagatgataccatcccttgtggtacaatggaccattaggatcgttcatccaatttttttgaggttgaaaatggtaagcagttctttgccagctaagcatagcattggaccacgcataagaaacgtgactagcattgacgacatctcgaaaagtcttatcggagactccctgagaaacacctcttgacggcggcgccggcgaacgggagttactctgcaagtccggtgactggttgttgaggatcggaaagaaggctacagaaagcaaaaggaaagaggagaggaaaatgccggagatgattttaagggacttccggtggccggaatcgggttgatccgggaggaatgtggatccgttaacaagcttgttatcaactgatctctctactagagagtgtgtggtagagtgtgattgcacttgaattacagctgagatttttagttttataatactgatatcaagggtaactttatagacctgattttgtcggaatttacgagaggaccataggtatgattggtacacacgtcttgtgattgagggtattttggggatttaagcatttcttgtgattgaggctattttggggattgaaatatgacttttgtacttagcttaaaaaaaatatctaatgatgaaaaagaatatactattagaatcattatttttcgaaaaacagttactcaattttaatttgtttgaactacgttatttttcaaaaataaaattaaaagatattttgatatatttaacttatttttaatttaaaacaataaaaattcaataataaaaaaattcttatcaaattaaataaattaaactgaacaaagtattcaaaagattatttattagtgaacgtcatttcacttgtttgcacgagagttagatatgatcacatacgcttgagagaataaaaaaagagtggcggacgtgtataaaccgttgatgattaaatgtgattattagtgagaaaatgaattgtttaatcaagattttttgtatttaaataattggtatgatcacatgcgcttgagagaataaaaaaagagtgacggatgtgtatgaaccgttgatgattaaatgtgattattagtgagaaactgaattgtttaatcaagattttttgtatttaaataattggtatgatcacatgcgcttgagagaataaaaaaagagtgacagacgtgtatgaaccgttgatgattaaatgtgattattagtgagaaaataaattgtttaatcaagattttttgtacttaaataattggtacttgcagacataggggaggaaatgaaatgacaaaagagatttggaataattcgaaattgccagaattgatattatagtttatactatgctgtcactttcccaatgaaagttggtgtatcaagtttcaagaaaataattataacataatgatgatgactaaagacccatgttgctatgcttaaatggacgatcaaatttcatcgtccgtccaatttaccagtgatttcacagaacatcaatcccgtcatacttcttttctaattggttttcaaattacacatcgtttatttattcttaatcaaatagtgcatgatatgtttgacacacaaaactaaatattggacgtaagtaaatttattcggcgattcaagtaaaaagttggcatttatacttaactaatggatgtatgatatcacatgtgtttgaacatattaacttgatgtaaacaccgaatacgagtaaatacttatcaatgaaactgaccatactctttccacgagccgagcggtgtgccatttatgtccatctgccgacagccctggcacactgcggtgcaagaaacatgcaaggcccaagggcacatgaaaaaggagaggaaactcacatgctatgaaataaattcacatttgacaactccataaacagaaaatgcaaaaaaaaatgtctttatgtaccgaatttacttatttttgcacagatattatcctttcattaaaaggaacaaactaaagaacactcgatattaagaacaccaaactacaaataaacaaccataggaacatgataggtttgcgaacaataaatgcccattttcaagtacaacaaccccctccttgtgaagcagctccgtctcttccacctgaaggcccgggaatgcctccatatccaacttttattccatacgactgcaaaagaaaaaagggaagcgtaagtttcggaaaccatctattattacgtaaatgataattatacatgttggaaatcaattatcactgagatatacaagtatttactgaaatatttgcgacgtaagtttttgttttcttggttctcaaaacaataattaggacactaggattgttccaaacagaactgaaaccgataacccgaataaaaaaagttattggtttatcaatattgggttattagtttagcggttttgataacgatttagattttttttgttatcgggttatcggttcttaacggtttgaagttttttcttaacgggttaatcgataacccgatagtaaattaaataattatatttataccattttgtatataaagtctttgacttagagtttggtttcccactttcagttttggttgtctcagacacttctcgagggtaccacattgattgagttttatatgcaatatagtaataataataatatttcttataaagcaagaggtcaatttttttttattataccaacgtcactaaattatatttgataatgtaaaacaattcaattttacttaaatatcatgaaataaactatttttataaccaaattactaaatttttccaataaaaaaaagtcattaagaagacataaaataaatttgagtaaaaagagtgaagtcgactgactttttttttttatcataagaaaataaattattaactttaacctaataaaacactaatataatttcatggaatctaatacttacctcttagaaataagaaaaagtgtttctaatagaccctcaatttacattaaatattttcaatcaaatttaaataacaaatatcaatatgaggtcaataacaatatcaaaataatatgaaaaaagagcaatacataatataagaaagaagatttaagtgcgattatcaaggtagtattatatcctaatttgctaatatttaaactcttatatttaaggtcatgttcatgataaacttgaaatgcgctatattagagcatatattaaaataaaaaaatacctaaaataaaattaagttatttttagtatatatttttttacatgacctacatttttctgggtttttctaaaggagcgtgtaagtgtcgacctcattctcctaattttccccaccacataaaaattaaaaaggaaaggtagcttttgcgtgttgttttggtacactacacctcattattacacgtgtcctcatataattggttaaccctatgaggcggtttcgtctagagtcggccatgccatctataaaatgaagctttctgcacctcatttttttcatcttctatctgatttctattataatttctctcaattgccttcaaatttctctttaaggttagaaatcttctctatttttggtttttgtctgtttagattctcgaattagctaatcaggtgctgttatagcccttaattttgagttttttttcggttgttttgatggaaaaggcctaaaatttgagtttttttacgttggtttgatggaaaaggcctacaattggagttttccccgttgttttgatgaaaaagcccctagtttgagattttttttctgtcgattcgattctaaaggtttaaaattagagtttttacatttgtttgatgaaaaaggccttaaatttgagtttttccggttgatttgatgaaaaagccctagaatttgtgttttttcgtcggtttgattctgaaggcctaaaatttgagtttctccggctgttttgatgaaaaagccctaaatttgagtttctccggctgttttgatgaaaaagccctaaatttgagttttttccccgtgttttagattgtttggttttaattctcgaatcagctaatcagggagtgtgaaaagccctaaaatttgagtttttttcgttgttctgattgttgtttttatgaatttgcagatgcagatctttgtgaaaactctcaccggaaagactatcaccctagaggtggaaagttctgatacaatcgacaacgttaaggctaagatccaggataaggaaggaattcccccggatcagcaaaggcttatcttcgccggaaagcagttggaggacggacgtactctagctgattacaacatccagaaggagtctaccctccatttggtgctccgtctacgtggaggtggatccatgaagcggtacaacttgcatacggcataggagaaactgaaatgccaactgtgatgaatgggcttatggtttggtgcattgaaaatggaacctcgccaaacatcaacggagtttgggttatgatggatggagatgaacaagtcgaatacccactgaaaccaatcgttgagaatgcaaaaccaacacttaggcaaatcatggcacatttctcagatgttgcagaagcgtatatagaaatgcgcaacaaaaaggaaccatatatgccacgatatggtttagttcgtaatctgcgcgatggaagtttggctcgctatgcttttgacttttatgaggtcacatcacgaacaccagtgagggctagggaagcgcacattcaaatgaaggccgcagcattgaaatcagcccaacctcgacttttcgggttggacggtggcatcagtacacaagaggagaacacagagaggcacaccaccgaggatgtttctccaagtatgcatactctacttggagtcaagaacatgtgtaacttttactcatctcctccaattatttctgatttcatgcatgtttccctacattctattatgaatcgtgttatggtgtataaacgttgtttcatatctcatctcatctattctgattttgattctcttgcctactgaatttgaccctactgtaatcggtgataaatgtgaatgcttcctcttcttcttcttcttctcagaaatcaatttctgttttgtttttgttcatctgtagacatgttcttgactccaagtagagtatgcatacttggagaaacatcctcggtggtgtgcctctctgtgttctcctcttgtgtactgatgccaccgtccaacccgaaaagtcgaggttgggctgatttcaatgctgcggccttcatttgaatgtgcgcttccctagccctcactggtgttcgtgatgtgacctcataaaagtcaaaagcatagcgagccaaacttccatcgcgcagattacgaactaaaccatatcgtggcatatatggttcctttttgttgcgcatttctatatacgcttctgcaacatctgagaaatgtgccatgatttgcctaagtgttggttttgcattctcaacgattggtttcagtgggtattcgacttgttcatctccatccatcataacccaaactccgttgatgtttggcgaggttccattttcaatgcaccaaaccataagcccattcatcacagttggcatttcagtttctcctatgccgtatgcaagttgtaccgcttcatactagtttttaatgtttagcaaatgtcctatcagttttctctttttgtcgaacggtaatttagagttttttttgctatatggattttcgtttttgatgtatgtgacaaccctcgggattgttgatttatttcaaaactaagagtttttgcttattgttctcgtctattttggatatcaatcttagttttatatcttttctagttctctacgtgttaaatgttcaacacactagcaatttggctgcagcgtatggattatggaactatcaagtctgtgggatcgataaatatgcttctcaggaatttgagattttacagtctttatgctcattgggttgagtataatatagtaaaaaaataggagctcatcgttatgctataaatttcatatatttagttgggagtaggctttataccgagttggactacggtcagtcactttcaagtcctagaactacgtgcccctgtaggttataagtctcctctgtgggcatcaatttagtgatcatgccagtcatgcctctatacctctgacaggatatatggtactgtaaacactagttgtgaataagtcgctgtgtatgtttgtttgagatctctaagagaaaagagcgtttattagaataatcggatatttaaaagggcgtgaaaaggtttatccgttcgtccatttgtatgtggtcacctatctcgagcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaaaacgaactccaattcactgttccttgcattctaaaaccttaaataccagaaaacagctttttcaaagttgttttcaaagttggcgtataacatagtatcgacggagccgattttgaaaccgcggatcctgcagccaaagcacatacttatcgatttaaatttcatcgaagagattaatatcgaataatcatatacatactttaaatacataacaaattttaaatacatatatctggtatataattaattttttaaagtcatgaagtatgtatcaaatacacatatggaaaaaattaactattcataatttaaaaaatagaaaagatacatctagtgaaattaggtgcatgtatcaaatacattaggaaaagggcatatatcttgatctagataattaacgattttgatttatgtataatttccaaatgaaggtttatatctacttcagaaataacaatatacttttatcagaacattcaacaaagtaacaaccaactagagtgaaaaatacacattgttctctaaacatacaaaattgagaaaagaatctcaaaatttagagaaacaaatctgaatttctagaagaaaaaaataattatgcactttgctattgctcgaaaaataaatgaaagaaattagacttttttaaaagatgttagactagatatactcaaaagctatcaaaggagtaatattcttcttacattaagtattttagttacagtcctgtaattaaagacacattttagattgtatctaaacttaaatgtatctagaatacatatatttgaatgcatcatatacatgtatccgacacaccaattctcataaaaagcgtaatatcctaaactaatttatccttcaagtcaacttaagcccaatatacattttcatctctaaaggcccaagtggcacaaaatgtcaggcccaattacgaagaaaagggcttgtaaaaccctaataaagtggcactggcagagcttacactctcattccatcaacaaagaaaccctaaaagccgcagcgccactgatttctctcctccaggcgaagatgcagatcttcgtgaagaccctaacggggaagacgatcaccctagaggttgagtcttccgacaccatcgacaatgtcaaagccaagatccaggacaaggaagggattcccccagaccagcagcgtttgattttcgccggaaagcagcttgaggatggtcgtactcttgccgactacaacatccagaaggagtcaactctccatctcgtgctccgtctccgtggtggtggatccatggacctgcatctaattttcggtccaacttgcacaggaaagacgacgaccgcgatagctcttgcccagcagacagggcttccagtcctttcgcttgatcgggtccaatgctgtcctcaactatcaaccggaagcggacgaccaacagtggaagaactgaaaggaacgacgcgtctctaccttgatgatcggcctctggtggagggtatcatcgcagccaagcaagctcatcataggctgatcgaggaggtgtataatcatgaggccaacggcgggcttattcttgagggaggatccacctcgttgctcaactgcatggcgcgaaacagctattggagtgcagattttcgttggcatattattcgccacaagttacccgaccaagagaccttcatgaaagcggccaaggccagagttaagcagatgttgcaccccgctgcaggccattctattattcaagagttggtttatctttggaatgaacctcggctgaggcccattctgaaagagatcgatggatatcgatatgccatgttgtttgctagccagaaccagatcacggcagatatgctattgcagcttgacgcaaatatggaaggtaagttgattaatgggatcgctcaggagtatttcatccatgcgcgccaacaggaacagaaattcccccaagttaacgcagccgctttcgacggattcgaaggtcatccgttcggaatgtattaggttacgccagccctgcgtcgcacctgtcttcatctggataagatgttcgtaattgtttttggctttgtcctgttgtggcagggcggcaaatacttccgacaatccatcgtgtcttcaaactttatgctggtgaacaagtcttagtttccacgaaagtattatgttaaattttaaaatttcgatgtataatgtggctataattgtaaaaataaactatcgtaagtgtgcgtgttatgtataatttgtctaaatgtttaatatatatcatagaacgcaataaatattaaatatagcgcttttatgaaatataaatacatcattacaagttgtttatatttcgggtggactagtttttaatgtttagcaaatgtcctatcagttttctctttttgtcgaacggtaatttagagttttttttgctatatggattttcgtttttgatgtatgtgacaaccctcgggattgttgatttatttcaaaactaagagtttttgcttattgttctcgtctattttggatatcaatcttagttttatatcttttctagttctctacgtgttaaatgttcaacacactagcaatttggctgcagcgtatggattatggaactatcaagtctgtgggatcgataaatatgcttctcaggaatttgagattttacagtctttatgctcattgggttgagtataatatagtaaaaaaataggaattctatccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggaagctt

SEQ ID NO: 2 - DNA, 29956 bp, synthetic construct
  misc_feature 1..29956: note = pSIM4363 T-DNA
  source 1..29956: mol_type = other DNA
tggcaggatatataccggtgtaaacgaagtgtgtgtggttgatccaaaatctatcgtacctttagaaagtgtagctatgaaggatagtctcacttatgaagaactacctattgagattcttgatcgtcaggtccgaaggttgagaaaaatagaagtcgcttcagttacggctttgtggaggagtaagggtaccacattgattgagttttatatgcaatatagtaataataataatatttcttataaagcaagaggtcaatttttttttattataccaacgtcactaaattatatttgataatgtaaaacaattcaattttacttaaatatcatgaaataaactatttttataaccaaattactaaatttttccaataaaaaaaagtcattaagaagacataaaataaatttgagtaaaaagagtgaagtcgactgacttttttttttatcataagaaaataaattattaactttaacctaataaaacactaatataatttcatggaatctaatacttacctcttagaaataagaaaaagtgtttctaatagaccctcaatttacattaaatattttcaatcaaatttaaataacaaatatcaatatgaggtcaataacaatatcaaaataatatgaaaaaagagcaatacataatataagaaagaagatttaagtgcgattatcaaggtagtattatatcctaatttgctaatatttaaactcttatatttaaggtcatgttcatgataaacttgaaatgcgctatattagagcatatattaaaataaaaaaatacctaaaataaaattaagttatttttagtatatatttttttacatgacctacatttttctgggtttttctaaaggagcgtgtaagtgtcgacctcattctcctaattttccccaccacataaaaattaaaaaggaaaggtagcttttgcgtgttgttttggtacactacacctcattattacacgtgtcctcatataattggttaaccctatgaggcggtttcgtctagagtcggccatgccatctataaaatgaagctttctgcacctcatttttttcatcttctatctgatttctattataatttctctcaattgccttcaaatttctctttaaggttagaaatcttctctatttttggtttttgtctgtttagattctcgaattagctaatcaggtgctgttatagcccttaattttgagttttttttcggttgttttgatggaaaaggcctaaaatttgagtttttttacgttggtttgatggaaaaggcctacaattggagttttccccgttgttttgatgaaaaagcccctagtttgagattttttttctgtcgattcgattctaaaggtttaaaattagagtttttacatttgtttgatgaaaaaggccttaaatttgagtttttccggttgatttgatgaaaaagccctagaatttgtgttttttcgtcggtttgattctgaaggcctaaaatttgagtttttccggctgttttgatgaaaaagccctaaatttgagtttctccggctgttttgatgaaaaagccctaaatttgagttttttccccgtgttttagattgtttggttttaattctcgaatcagctaatcagggagtgtgaaaagccctaaaatttgagtttttttcgttgttctgattgttgtttttatgaatttgcagatgcagatctttgtgaaaactctcaccggaaagactatcaccctagaggtggaaagttctgatacaatcgacaacgttaaggctaagatccaggataaggaaggaattcccccggatcagcaaaggcttatcttcgccggaaagcagttggaggacggacgtactctagctgattacaacatccagaaggagtctaccctccatttggtgctccgtctacgtggaggtggatccatggcggctgctgcctcaccatctccatgtttctccaaaaccctacctccatcttcctccaaatcttccaccattcttcctagatctaccttccctttccacaatcaccctcaaaaagcctcaccccttcatctcacccacacccatcatcatcgtcgtggtttcgccgtttccaatgtcgtcatatccactaccacccataacgacgtttctgaacctgaaacattcgtttcccgtttcgcccctgacgaacccagaaagggttgtgatgttcttgtggaggcacttgaaagggagggggttacggatgtatttgcgtacccaggaggtgcttctatggagattcatcaggctttgacacgttcgaatattattcgtaatgtgctgccacgtcatgagcaaggtggtgtgtttgctgcagagggttacgcacgggcgactgggttccctggtgtttgcattgctacctctggtccgggagctacgaatcttgttagtggtcttgcggatgctttgttggatagtattccgattgttgctattacgggtcaagtgccgaggaggatgattggtactgatgcgtttcaggaaacgcctattgttgaggtaacgagatctattacgaagcataattatcttgttatggatgtagaggatattcctagggttgttcgtgaagcgttttttctagcgaaatcgggacggcctgggccggttttgattgatgtacctaaggatattcagcaacaattggtgatacctaattgggatcagccaatgaggttgcctggttacatgtctagattacctaaattgcctaatgagatgcttttggaacaaattattaggctgatttcggagtcgaagaagcctgttttgtatgtgggtggtgggtgtttgcaatcaagtgaggagctgagacgatttgtggagcttacgggtattcctgtggcgagtactttgatgggtcttggagcttttccaactggggatgagctttcccttcaaatgttgggtatgcatgggactgtgtatgctaattatgctgtggatggtagtgatttgttgcttgcatttggggtgaggtttgatgatcgagttactggtaaattggaagcttttgctagccgagcgaaaattgtccacattgatattgattcggctgagattggaaagaacaagcaacctcatgtttccatttgtgcagatatcaagttggcattacagggtttgaattccatattggagggtaaagaaggtaagctgaagttggacttttctgcttggagacaggagttaacggaacagaaggtgaagtacccattgagttttaagacttttggtgaagccatccctccacaatatgctattcaggttcttgatgagttaactaacggaaatgccattattagtactggtgtggggcaacaccagatgtgggctgcccaatactataagtacaaaaagccacaccaatggttgacatctggtggattaggagcaatgggatttggtttgcctgctgcaataggtgcggctgttggaagaccgggtgagattgtggttgacattgatggtgacgggagttttatcatgaatgtgcaggagttagcaacaattaaggtggagaatctcccagttaagattatgttgctgaataatcaacacttgggaatggtggttcaactggaggatcgattctataaggctaacagagcacacacttacttgggtgatcctgctaatgaggaagagatcttccctaatatgttgaaattcgcagaggcttgtggcgtacctgctgcaagagtgtcacacagggatgatcttagagctgccattcaaaagatgttagacactcctgggccatacttgttggatgtgattgtacctcatcaggagcacgttctacctatgattcccattggcggtgctttcaaagatgtgatcacagagggtgatgggagacgttcatattgaactagtttttaatgtttagcaaatgtcctatcagttttctctttttgtcgaacggtaatttagagttttttttgctatatggattttcgtttttgatgtatgtgacaaccctcgggattgttgatttatttcaaaactaagagtttttgcttattgttctcgtctattttggatatcaatcttagttttatatcttttctagttctctacgtgttaaatgttcaacacactagcaatttggctgcagcgtatggattatggaactatcaagtctgtgggatcgataaatatgcttctcaggaatttgagattttacagtctttatgctcattgggttgagtataatatagtaaaaaaataggagctcgattctttgaaaagaggcttcatactccctccgttcatcttcacttgttcattatattatttaagaatatttaataatatttatccaatttataaaatcaatgaataatttatcattttattcttgttattaaatattatttattttccatattcagaattatggagtattaaatgaaatatttagttcatgaattaactagtacaaatttttttaaaatcaccttatcgagcatattaaatgtaaataattaacgcagcaaactaacctatgagattttcatattctcagctttgcaagccgtttggaaagacttattggggaacgtagtcttacaagtaacagcaatagtgccgagatcaccaaatgcattgtgctcagtgtgagaggttgcacgaagcttcttagtcaagcatttctcatttgtatatcttttacgattctatctattaatgatttttgtatacatttgattggcatatttctatattttccaacccataggtatttgtataatattttatatttatgcaaatcatcatataagcaagaaattcgaatcaaagagcaaatatatcaaaaatttcaaaaagataccatacatgcaccacaaataaggtctacttttttctattgaaaataagatttttattttattactccattaattaaagataaataaacataaaataaaacaaagagaaatataaaagtaatttgtttttacaaatcacaagcaattaacttgaaacagtaatcaaactttttctcttattattagtattataataccaacaggcatcagaagattctctccaacacagaatcattttcaaaggcatccctgcaatttattagacatggaatatttagttgatgaactaaatacaaatttaaaaaaatcatggatattaaacgtaaataattatttatagtacctgtgatattctcaactttgcaagccgttccgagaccctgagactaattggggaaatgtttgggccttgtacaagaaatagctttttcaatttaggcatactcatatcatccatgcacgttacttcagacaacccttgtgcatcctcaatatacaaaacttcaagttgcggaaagctatcaccagagagatccattttttctgcatcatgtctgcaccaaagcaatataagtatccttaatttgggcaatttcttaagcactgccaatagatgaccgtctaccatcaagccgacaagattaagcttgacaagatttggagggaatgcttcaatgttcaagagcattgctgagaaaccaaccaaacccaactcgacaatatttggatgggacgacaagtttatttgctcactcgtgttcttgaaaaatctgagcttcagaacctccaacgctctaggcacagggctcaatgctgataatatcttaatggtagaaccggatacatccattatacacaatgttcttaaatttatcaatcggtgcaacaatcttggttcacaaaatttatcatccacccacatcaaagtttgtagattattaggaggcatgattcggcaaaatggacttgcaaaagagacacagtaacattcttctgtgtaacaaagatgtctcaatattttagactcccaaacaccaaaaggaagtttactagagctatgaaatatatccagggtctctagacatttgagcttgacaatactatttggcaattttactctaattgccccctccaatcgcacataacgtaggcaactcatgttcccaatttttttggggaaagtaacatgctgataacccttttgagacatgactacaaccaacacttgcaataatttgaagttaagatgagccatttgagaaaatatgcaacggtcttttgtgaaacagaaaagtgaacgaagcttcataggcttaggatttaaatggaagaactcattcatggcattatcatcagagtagaatgtaatccttcgcaccctagcaacattgctaggatcaccaaatgcattgtgctccgtgtgaaagaagttactttccttagccaagtccacacacaaactatgtaacaagtcatgtatgcgacaacttgaaattcttccatcatatgtccttttggcaacttgaatcaagtttcttgaaaccaaatcatttaggacatcatccgccaaactttcagcctctcgcccattgccagtatttacaactatcagcttctcagcaatccacatatttgtcaaatcaaaagcacgaatttcatggtcctcggggtaaagaccaaagtacaagaaacatggccttaatgcaatgggcaaatcattgtaactcagagccaataccttaccacatccatcttgaattttatgagccatactctcaagtactctgttccatgcatgttctgttcttcctcttgcccttaacatgcctgcagtcaccacaattgctagcggtatacctccacatctctcaactatacatctaccaatatttaccaagtctggtgaagcattggcccaattatcattaacaaaattaaagattttcttggtaaagagttcaaagcttttctctgaatctaggggttgcaacacgtggattgagaaatcccctcctatgtatctgcctacattactatttcgagaggttataattatcctactgccaatttttgaatcacattcaggaaggacaagttttagatcatcccaaatttcaacatcccaaatgtcatctaagagaataacatacctttttattttcaagagtgatcgtaggttgttctccaagttttctttcctttcctcttccgtcagtccaacttgtttggctatgtcatgtaagatttctcccgcccttggctgttgtgaaacatagaccagtcccgaacactcaaattgatgacagacatgcctataaagtttcttggcaagagttgtttttcccaaaccgggcatgccaactattgaaacaactccataaggcaaatcatgatcaagtaatttggcttgtagtgtattgaagtcatcttccagaccgatgacctctgtttcatcagcatgaaggaacaatcttctccggtccaatggaatgcaatcatcattattgttacttgtatctgtgatgctgtaagttgtccttacacggtcaatatcagcaactcttctttttatcttctcaatctccatagcaaactcatcggcaaaagaaaccgtcttaaggcaacaaatgaacttattggattgttgaatttttggaagaaactcatctaatagatcctccacatcacctgccagttgttgaatatcttttaataagtttttcacccttgaatcgcctccaacttcctttgcctttgcattgtctacatatgatcgaatgtgtctcatttctctctggagccaatcgatgtcctctttcaaccaatataaacgcgtaccttcttgaaagagtacatttccagctatttctattgatttattgatgactgctgtgagaagaatttcagccatctcctttaatttttcgttaaatttcttttttctaaatgtgaggatgaggaagggaaagtaagagtcaacggcccaagtcttgtaaacacaataattcatcttttgttagctggtatttcgttttcaagcatgaataacaaacgttgaaagtcagaaaattcaggcagtactacgcgtaaaacgaaattagaaagaagaagataattggtaaaggaaaagaaaggtcagcttagaatagttggagttcagggccaagtctcctaataatgtcaagtaaatgaaaaacgaaagaagaaaggaacaacacgtgctgtggatagatactagtagaatagtcaaaagaaattacaccgttcgtcccaatttaagtgtcattcaattaaattcgacatggaatttaaaaaacaaacagtaacttttaatattttgtgactgatgattgataatgtaccttttttttttgaaaaaattacgtaactaagcaaacatctttagtatataactatcaatcacaccatatgttaaaaaaatttattatatatctagcgccattttatgtttcctaactgtttgatgaacgtcttgcaaaactttacccaatttgggttcatggctgtgagtttgggctattatgagactcaatttgtatgttccatgaaaattaggcttttatgcaacttgattctctcaaaatcttttgccatactcatacagagtgggcgttcggttcgatatttatttaactttgatttgatttggtcgtgagacatcatcataatgactcgagtagaatgtagggtgtataactgggcccgtccatatgtggaagctactctctttgtccaaaaatagttgtccactattgacttacacacaccttaagaaataataaataataggggtactattactacactatcctttgaatattataaatttaatgctctgggaaaatgtattagatattgaatactccctccgtttcaaaaagaatagtctactttgacttggcaaagagttttaaaaaataaagaaaatttttcaatcttgtagttctaaattaatgttatgtcaaatgtaccaaaatatcttttaatcttgtagccttgaacatgccacgtggaaagttgaaattaaagtgttgatcaaaaaggaaagaggtcactattttctaaacggaggggtggtatttaattgtaggggtaaaatagacacaaaaggataaattatcttttgattttctaaattggacaagtattgttggacaactatttttcgtatagtgggcaagtaaagatggacgggggaggtattaccaagtctaaaaatgggaagctatggccaagtctaaactgatactccatgtgttcctttttatttgtcacgtttttttttttttgagaaatcaaactacatgaattttgaccaatattttaagatctactttttcaaatagttctcaaacatctaaattttaaataataaattattctaattcaaattagcttcaaaaattagtcaaattgactttcagaaagtcaattgtgacgattaaaaaagaatggagagagtaagtttttttaaaatcttattctccattatttacatgacgagtcttggagaattttcactttgttaaggattcgattcttcatatcgtaatcccttcccctattcttcctttcacctgcccctttttgaaaattaaatatgacccagaggctgcttttttatatattgcttcacataccccatttttttattttttattttgaaaaacggaaaaagacatgacgagccccactagtctttcattatttctcattttttgtacacattgaatttctttggaatttaataaaggtagtacttagatttgaccaatccaaactatgtgcctttggcgtgttgctttttttaaaaaaattattttttgaaacttttgataataagttgtcacatgtgtagcatttttgactttgatcttcaaggcttattgagggcaagtggggtcgaaaattcaatatcaccgatttataattggatggggatttttatttacatctggcattagtcgattttgaaaagaatgtctctttcgtaatttgagaactcgttaatcacaatattgcacatcaacagataacatgtttaagatcacaaaattaaaaaacaatttgatatattattacacatatctttagtttaacactataagattcaaaaatcttctttggtttctgaaacttcattgtcgaatcaaataaaaacgaaaaagttgaaatagagagagaaaaggtagtttccttgaaatttctctagatttggagcaatccaatgtttttctgtatcaaaattttacatattgatggtgagtactgtcaataatgtagtactccctctgtcccaatttatatgactcactttcctttttggtcagtcccaaaaagaatgacacatttctacatttagtaacaatttatttttaaaatgacttttttactcttaatgaaatgatttctagccacacaaatatctaccactcattttagaccacaaatttcaaaagtcttcctttctttgttaaactccgtgccgagtcaaactatgtcatataaattgggacggagggagtatgatatttcttctcttttgagtaatatttaatatcttcaattaaatcctatccactcctcatctattcttgttcatcatttttgagaattaatttgatggcagcttatagtgctgtaatttctcttcttcaaacacttattgatcaacaaaatatttcagaactctttcatggtcacactgcccaaacgctcgattctcttcacactacagctgaatattttcaacatgtccttgaaaatattacaagatttgactctgaaaagatcaaatctttggaggaaaaaataagagttgttgttagctatgcagaagatgttgttgcaatgaaaatttctcaaatcatcataggctcaagctggacatttggaattttacaacaccaggatttactaccacttgttgaaaaaatggacacaacaaagaaacaagtgatggacattctttctcatgatgatgatcaaattcttgaattaactgcaggggattccttgattggcacttcttctacaacttatccaatgttggaagatgatatcgtgcagggaattgatgatgacttggagatcatagttaaaagattgacaggaccaccacgggatttagacgttgtcacaataacaggtatgggtggcattggtaaaacaacactcgctagaaaggcttatgatcatctcacaatcaggtatcactttgacattcttgtttggattacaatatctcaagaatttcgatgtagaaatgtattgttagaagctttacattgcatttcaaagtcaacagatattgtgaacacaaaagattatgataagaaggatgacaatgagttagctgacatagtacagaaaaaactaaagggtccaagatacctagttgttgttgatgatatttggagtagagatgtttgggatagtataagaggaatatttcctaattacaacaacgggagtcgaatcttattgactactagggaaaacgaggtagcaatgtatgcaaatacttgtagccctcatgagatgagtcttttgagtttagaaaacggttggaggttactttgtgataaggtgtttggaccaaaacatgatcatcctcctgaattggaagaaattggtaaggaaatagttgaaaaatgccaaggactacccttaacaatttcagtgattgcgggacatgtttctaaaatgcccaggacattagaatgttggaaggatgtcgcccgaaccttaagtgaaatcatctctagtcatccagataattgcttaggagtgctcggtttgagttaccatcacttgcctaatcacctcaaaccttgctttctttctatgagtagtttcccagaagattttcaggttgaaactcggagattgatctacttatggatcgcagaaggtttcataaggacttgcgaaaatggtaaaagtttggaggaagttgcagtagattatttggaggaccttattagcaggaacttgatacaagctagaaaaaggagattcaatggtgagataaaagcatgtggaatacatgatctactgcgtgagttctgtttgatagaagctgaaataacgaagcatatgcatgttgagagaacttacccaactcttccaacacaaaagaataatgttcgtcgcttcagttttcaaacaaaattttattcagttgatgattgtaataagctattaccacctgttgccagatctatctacttcttttctcagttggatctacctgttgtaccctataagaggtatctcaggtgttgtttgcccatccaccgtgatgatcgtataatacatgatttttactctcgtttcaaccttctcagggtattggtcatctccaagacaaatgaatacttcgagtcatttccacttgtgattacaaagttgtttcatttgagatatctccaagttcgatttcttggagacattcctgaatcaatctcaaaccttcaaaatttgcaaactctaatttgtagtggtggtactttacctgggaagatatggatgatgaagaacttgaggtatataagtataataggcaacaaagtcacttatttaccaagtcctagaacagaaagtcttgtgaatctagaggagttttctgttctttgttacagaagttgtacaaaagaagtcatttctggcattcccaatctaaagagattgaccattgatgtactttctagcattaacaactattttcccaatggactaatagatatgtccagcttgacaaaactcgaagcattcaagtgtaataggtgtttatattcgaatttcaatagttctgttattccaacatcacttaaggattttgtttttccaacatcacttaagaggttgagtttaaactattatgctagtcattttttttgggaagaaatatcatcaactattatcatgttgcctaatcttgaagagctgaagcttaaagattgtcgatccgatgaatatgatgaatggagtttgagtgataaagacaaattcaaaagcttgaagttgttggtactaaccgacattttttttgatcgttgggaagctaccagtgataacttcccaaatctaaaacgccttgttctgaacaagtgcgacttagaaattccatcagattttggggaaatttgtactttggaatcaattgagttacatgattgcagcactagtgctgaggattctgcacgagagattgaacaagaacaagaggagatgggaaataatatccttaaggtctacatacatggcagtcgcagtaagttctaaacagttattcgattctctcatctttgttaaatcttacctccttgaagttatgatgatgacttcgattttttgtcttgcataggcaggaaaatggaagcatatttcgaggaatcatctaatgccatcttccaaatgcagaaccttttagattaatcttttatgttctgattgtttgttaaattcatgttttaggaataaacacgtttaatacatgtttcgctctttctttactatgtaaacagtaactattgatgtattgttatatctattttgtgttatgtgatagttcgaaatgaataatgtgttggggttttggtctttttcccttcctatgtggataaataaaagcccaattggtctagcccacttatgcccataagcccaatttatggagcatacataagactttctttaagtcttattttcatatatcacacataatttttacaagagagataagcatacatattagagagaaaagagagaaagtgcagattttcgcacaaaccctagcagccaatttcaaatcgcgattcccgcttcgtttcttatctgattaagctgatttttggacagcacgttcctctcaacttaatctttgattgagaactgacagaggtcgatttggagtcccgtagctccagatttttgctcgtgaacagcagctgctatttggtgattttgctccttttacttctctagttctttggtgctatcttgtagttgttgttgctcgtttttggcacttgttttggtgaccattttggagaggatcccccggggatctagaatttaagctttatggatgtaatattatttttaaagttatatgttcacatctattatttgttataattttcatgaaaatttatatatatatattttgcatgtatgttgcagtgaatatcttagattagtgtattttattatcagggtttttgtaatatttcatttttttatgtgggcacctaacatagctatagagagctttctttcaagttctcacgatcggacgtataaaaagtacctacataaaactttactcaggagaaagaagaggaaagcagtagaaaaagaatacaagaaactgaagatcaaaagcagcataaaaagtcaaaacctctcactacaaatatctttcctatttttatgcctcatatattattttatctaccctccccatcatgtttcattccttcaaaccatattatataggaaactcaattttgtaaatatgtacgtctacctggctaatattcatattacaagttgactaagacttgtggagattgtcagcttgtgaatgaagatgatgtctgatgcaactttggtgttgtggtttatgagaattgacgggagcctacgagcaaccataaagttgtctccatgtaacctatagatcatgggtttgagcggtgaaagaagccactaatttttctacagtatcctcctgggatgcgatccctccattaatcctaccaacgttggatgttttgtgcatgaggctgctttgtttacgggacattcctgatgtttcttgtttttcatagttcccctttagataaagtaaacaagagagtttgaagatgaacttaccttttgacagtctttagtttagcaatattagaagcccttgtttgagaaataatggcaaagagagttgtttgctaaatgaagtatagttgaatgataaattttatatacctagaaacaaggatcaaattcccacttcatacttccttttttctcctttcattagtggtgcattcatgctctagaaattctagattcgcttctgaggtgtaaatttgtattagcagtaaaaatctcaaagaggaatgtacaatctcttagagtcgagatacacctacacatcttgcttttactgttctatcgggagttacttcatgtattgcttgacataaaactgattgtggattcgtcttggtagatatccaggtaatacatcctgtctgctctagccacactaaccatatgcatcaggcttgaaatcgccctccgggatagtatgtgcttgtcaacttagagttggaaccaccatctgctgactgctcaaacttcacctcattaagatatttttcgagtttgtcatcaatcctatactttatgaatttaaagttgccacctgcagaaaagaagtcacaaatagaagaatattagtgattaaaatgagagtctattaaaaattgaaaaagaattaactaggatgaagaatgttgatattcatgacaaattcaatacttctgtagcttgtggctctaacacatttcagattctcaaaagtgtcttcctcccccatgttccattcttctccctgaatgattgtataataaaggattcctctccaacctccagtcttgctattgttgatagtgcatcggatgtcagaggaaagtcatacaacaacaattttttcaaattcgagattagagactgcccctttggcgatgaggctctgttggctaatgctgcaaagctggagacaatgcgatccctttggatgtctaattgttcagtaagttttgaagcatgtaagctgttggtccagaagttgccagggcttaatgttgaagttatagacgagaggggtcatccagatacgagaccagaaagttgccctgttgagaaactttacatatacaggacagtgtcaggaaggaggttcgacactcggattattgatgaagatgcagcattgacttgttcttctgttgaaatggtgctgtcaatgtacttgtggtcatgaattgtcgtaattctttttgaagattaatgtgaacctttcattcactttgagtgctggaaatcccatttgttgaacgtttagaagaaggtatttcagtgttctactacgagagttaatttgataaacaaacacttattttcgttcagagtgggatttcagcctacgttggttgaatgttcgagaaaaggaagtcatgagtgcttataagctctctacaacctcttcacttcctggcctagcttttgagatgaaagttcgttgggtttgtgataaagtagtatagatgtcattttaaatcagtaccttgattgaggttgaacgatagtgctctgagcatatgagacttcagaccaagttggacagccaaaggcaaatccacattgtatttataaaaactataatttttgttaaaaacttctgagttttctaaaagaaactcagaaaagtcaaataaaaatgtttcataacaactgtaacagaattttcttaaccatatacaatctacccagcaaagtgcaatctacttttcaaccataatgctcaaaaactacttaaataacgggatatccttctggccaaggatctgaagctcgtcccctcccctcatatcttcagcatattccttaatcttgagagcggaattttcaagttgagggctccttacaagttcgataattttcaaggaataaatatccccaaaactagacggaatctcctcaagattatgacagtccgacagttctaatttctcaagcgtgggaaaagattcctctccaacctcccacttggaaagaatcacttgactcaacatcaaacatttgagattctcaaaggtgtcttcttctcccatgttccattcttccccatggatgattgtacgataaaggtacaactcttcaaggttcagcagtctcgctattgttgatagtgaatcggatgtcagaggaaattcatgcaattgcaatcttttcaaactcgaaggaaagtgaaaatcccatggccgatttatggctgcagaggacccactgtcatttgtgtttgatctttcaaaatctacagtgagtttttctagttcagttaggaaatccaatttcgggaaccaatattgctctgttgaataatcccatgactccttgagtttgaaatgaagcacttgaagattgggaagccttttgaaaatatcctctgtatctttccaataggaaagcacgagttcccctaatgctgtcaagttctctaactttgtgtcctctgctatcagtattgattcatctgcatccatatcaaagaaagaacaagcagtcgtgaacagcacttgcaactttacaagatcccaaattctcggtaatagtatcaaggttgattctttgttatccacaaacaagatttctagattccagaggtttgagaaagacaaaggcagagatttaacttctgtcccaatgcttaagtacctcaaatgattcaacatgcatatttcattcagcaaagaatctttaaccatgataaaagaggattccaggtgcaaggttctaagaagcctcaagtgtcttagatgaaatgtatcagaaagatggtcgtccagctcatctccatttatggtcaaagaatagaggtgtttaccggaatgccttttcttatttgaaccgaacaggacaaaattaagcccaaagtgctcttcatcatcatcataatcaatgctaatttgacgtggcaacaaatctgatggagcacttgaacttatccgatcacacaacttttcctttcttgcttttatcaaacaaaagtcatgcacaagatcatgaagttggcaagtagggtaatcacctatctcattgaaacaaattaccaagctactggaaattaaatcatccaaataaattttcaccacttcttccagactcttcatatctgtcttttccacaaatccttcaaaaccccaaataagtttcaactcatggattgtcgttacccagtccttcggcgcacttgcaaagtacagcaagcatggcttcaggtgatcaggtaagtggtcataacttatttctataactttcatcacttccacttcattcttcaaaataaaggaatgcaaattatttacaacttcaagccacacactctttttcttttccctcccagcaatgattccagcaatcagatccaccaccaaaggaagccctttacaattttcggctatttctttaccaacatccaatagttcatcagggcaactctcgtttccaaatgcccttttctctaataactcccaactttcttctgatcttagcaatcgaaggttaagaggatcagtgtagagctttccatgcaaagcaactttcttttctcgagttgtcaaaataattctacttcctttcataccatcaggaaaaggtcttgttagctcatcccatgtattagtatcccacacgtcatctaagacaataagatacctctttccaaacaattgtttccgtagtttatcagcaacatcaatattctcactcaattttgaatttgagtcactaacttgattgaaaattttatccaacaacttcttctcgtcatatacttggtcgaccgtgcaccatgcacgaaggtcgaaatggctagaaactgatttatcattgtatactttgtacgccaaagtagttttacctaaacccggcataccaatgatcgaaatgacatctagatctgccggtccactggtgagctttctaagtatcaagtttgtctcctcaccaaaacctacaattattttatcagttgtcaatgacttgctctcaactggtttcttgggagagttcacaacgatgagacctctgttcttggaaatgttctcatgtaaatcagagacctcttctttgataagcatcatcttctttctggtaatgggaagtgagaaaataagatgtaagagaccattatctcgaacaataattgaatctatgacatcttttgcctcatatgccacatctagaacacgttcccagagatctttatacaatccttgctcaatattcgcgaaaaaagatcttatgaattccaagtcttctttcaccagcccaatttgttcctttatcaaagcaattgaataagcattggaatccagcaaatcatctaagtgtctctgtagcagatgcatgaagagaggtccatcactcatggggaagcaatattgagatgaatccgggactttcagataaacatgtttgagatcttccttaaggagttcaatattttccagaaactttagggttgcacaatttgtttcgtcagtactctctttaatccttaatttctcttccaagtcgcgtacaagagttgatacctccctggtaagtgctacaacacgagccaagagatcaaaaagtttgtcatgatgaataaagtccttgggcggcatatcagaaagaataatcaataggaattccatcatgacatgaatgtttcgagcccctgaagtgttaggggtaataacagttatcatatgctcttgtagatgaatcagatattctctgagaatgtccggagaggtttccaggagcttcttaatgaagcgtccaatttctgttgaagttgaagctttcaaagttttataacatatgtgcataacctccaattcagttggaacaatcttcaagagtagatgtgctagcttgaagagttgagggtctttatcattctgatcatcctcatctagctcggagagttgagagtcttcatcagcctgatcctcccaaaggaagcgtcctactctctcagccatcagttgaaacagagataagacattctcaaccatctcatgcttaatgcaacaattcactatcaatccatggaaatctcttatgttgccacatacattctgaagaacctcatattgagtcactccaggaaacatcttttcagcacgatgcttggatagatgagagagattcaagaggaggaagcccaattgctcatccatcatggtggcatctgatttagaacgatgatacaagcttatacagtcatccatattaccggcgaggctagtaaggacatatttacacccgacgtctttgccatcatcatccaaaattggttgaagcagattctcaacctcttgtctttttctagtcattatatcttcaaacttctccaaatcggaataagaaagctggacatatgtacaaataaatgtcagcttcaatttcaggctttcaatcagatccacatcaacagccttttgatcttcttcattctttaatctctctaggaaatccagaacattggcagcatccttgcgaagagcagaaaatgactcctgccaaaataacacatatcaaatttttaaagccacatgatatctacaattcaatactttacagttcactctatcaaataacataccaatgagttgtttgcttcttcattatcttttcgtttttccatgattctacttttcctttccaagttctcaagacctgatgcataaaaagtacataaaactttatcaggagaaagaagatgaaatgataagaataatcaataatttcactattttcacgcgaacttaagaggacttaattagtggaagatgataaaggtacacttgcgatttgaattgaagaaaatgacttgtaaaaatggggttcatccagaaacgagaccaactggtaccgaacctttctatctctaatagcgtatagtaatatattttcattagtgaggggaatctttcgtcatcttagaaaaaatctaaaacatctgcttctgttcactccacactctccaaccccatccctctcacccggtaagaattcacccgcaccggatattaataccaaattaatacaacttatcatgggtaagtaataatttaatgaggtgaaagtatacattaactaaccacaattaagtgatttgttgtaaatcggtggtgtgagtaagtatttaacctctctctttctcctgcaatttatttccctttctctttgttagatccaactaacctactaaagttctttcatttttcttacatcacttctacttttccataactgtacttcaatagagatgagagtagcaaatattagatgagttgactaacctttttagttgatgaaactatatatcctctgcttgctttgttattagatgtatagagctcaaaattctggaaataaacagaggaataattaagaccaaatatgatgcatagtattgttccaaaaagtcaagaaagcaaaagcatgaatcaattacagtggggcctcctcacttgtctgccacaaattaggaaaaagataaagaaaatgagaaaatagtcaaatgtcccctaacctatactcaaattttcaattacagacttaaatttcacaagggtctttgttaattaccatcgattttcaagacctccaatgttaattggatgtgtttttatttttaatttattatttgatttattcatatttattcattattaacacttttactgtgtgtctttacaggtacacatgtttgcttctcattgaagctattcgaatgaagttcgaacaacattctttatgttaagcagttagttaaatgcactgcagtgcactaacagaaaatgtacacctatcagtctagttgttagagtcagttaaattaactgcttcagattagaatataaatagagatatatagattgtaatagtatagttcttctcattatttttttcattcattttggttcatgagttcaatatagaatttttctctcttcttctccttcaatcagtctttctaaaatagttaattctctccattgtttagttagaattcactgaaattacatggtatcagagcatttgaagcagatctagggttttatctcgggattttttgagcatagatcgagagaatctgcaatggcgattgaagaggaatcagtggatgaaattcatggtagaactggaggagctgtaccgaggggaggttcggtgattctagatcgcggccgcgaaccatgcatctcaatcttaatactaaaaaatgcaacaaaattctagtggagggaccagtaccagtacattagatattatcttttattactataataatattttaattaacacgagacataggaatgtcaagtggtagcggtaggagggagttggttcagttttttagatactaggagacagaaccggaggggcccattgcaaggcccaagttgaagtccagccgtgaatcaacaaagagagggcccataatactgtcgatgagcatttccctataatacagtgtccacagttgccttccgctaagggatagccacccgctattctcttgacacgtgtcactgaaacctgctacaaataaggcaggcacctcctcattctcacactcactcactcacacagctcaacaagtggtaacttttactcatctcctccaattatttctgatttcatgcatgtttccctacattctattatgaatcgtgttatggtgtataaacgttgtttcatatctcatctcatctattctgattttgattctcttgcctactgaatttgaccctactgtaatcggtgataaatgtgaatgcttcctcttcttcttcttcttctcagaaatcaatttctgttttgtttttgttcatctgtagcttggtagattcccctttttgtagaccacacatcacgactagtcattacacattcctcccggatcaacccgattccggccaccggaagtcccttaaaatcatctccggcattttcctctcctctttccttttgctttctgtagccttctttccgatcctcaacaaccagtcaccggacttgcagagtaactcccgttcgccggcgccgccgtcaagaggtgtttctcagggagtctccgataagacttttcgagatgtcgtcaatgctagtcacgtttcttatgcgtggtccaatgctatgcttagctggcaaagaactgcttaccattttcaacctcaaaaaaattggatgaacgatcctaatggtccattgtaccacaagggatggtatcatcttttttatcaatacaatccagattcagctatttggggaaatatcacatggggccatgccgtatccaaggacttgatccactggctctacttgccttttgccatggttcctgatcaatggtacgatataaacggtgtctggactgggtccgctctcgagataattgtaactgatttattcttgaataacaacttcaatgaaatcaagcaacaaagctgatttcaacataaaaaaacagaacaagaaaacaaaaacagagcatcatccatcaaagtgtaatctcagcagattcaatagagactatctagagtgtatgggtgatccttctcttattataccgactaaagacattggtattaaggatatcttatcttttgaggagattcccgttcagattctggagcgtcaggttcgcaagttgagaaccaatgaggtaacatcagtcaaggtcttatggaggaatcagtagtctctattgaatctgctgagattacactttgatggatgatgctctgtttttgttttcttgttctgtttttttatgttgaaatcagctttgttgcttgatttcattgaagttgttattcaagaataaatcagttacaattattctcgagagcggacccagtccagacaccgttaatatcgtaccattgatcaggaaccatggcaaaaggcaagtagagccagtggatcaagtccttggatacggcatggccccatgtgatatttccccaaatagctgaatctggattgtattgataaaaaagatgataccatcccttgtggtacaatggaccattaggatcgttcatccaatttttttgaggttgaaaatggtaagcagttctttgccagctaagcatagcattggaccacgcataagaaacgtgactagcattgacgacatctcgaaaagtcttatcggagactccctgagaaacacctcttgacggcggcgccggcgaacgggagttactctgcaagtccggtgactggttgttgaggatcggaaagaaggctacagaaagcaaaaggaaagaggagaggaaaatgccggagatgattttaagggacttccggtggccggaatcgggttgatccgggaggaatgtggatccgttaacaagcttgttatcaactgatctctctactagagagtgtgtggtagagtgtgattgcacttgaattacagctgagatttttagttttataatactgatatcaagggtaactttatagacctgattttgtcggaatttacgagaggaccataggtatgattggtacacacgtcttgtgattgagggtattttggggatttaagcatttcttgtgattgaggctattttggggattgaaatatgacttttgtacttagcttaaaaaaaatatctaatgatgaaaaagaatatactattagaatcattatttttcgaaaaacagttactcaattttaatttgtttgaactacgttatttttcaaaaataaaattaaaagatattttgatatatttaacttatttttaatttaaaacaataaaaattcaataataaaaaaattcttatcaaattaaataaattaaactgaacaaagtattcaaaagattatttattagtgaacgtcatttcacttgtttgcacgagagttagatatgatcacatacgcttgagagaataaaaaaagagtggcggacgtgtataaaccgttgatgattaaatgtgattattagtgagaaaatgaattgtttaatcaagattttttgtatttaaataattggtatgatcacatgcgcttgagagaataaaaaaagagtgacggatgtgtatgaaccgttgatgattaaatgtgattattagtgagaaactgaattgtttaatcaagattttttgtatttaaataattggtatgatcacatgcgcttgagagaataaaaaaagagtgacagacgtgtatgaaccgttgatgattaaatgtgattattagtgagaaaataaattgtttaatcaagattttttgtacttaaataattggtacttgcagacataggggaggaaatgaaatgacaaaagagatttggaataattcgaaattgccagaattgatattatagtttatactatgctgtcactttcccaatgaaagttggtgtatcaagtttcaagaaaataattataacataatgatgatgactaaagacccatgttgctatgcttaaatggacgatcaaatttcatcgtccgtccaatttaccagtgatttcacagaacatcaatcccgtcatacttcttttctaattggttttcaaattacacatcgtttatttattcttaatcaaatagtgcatgatatgtttgacacacaaaactaaatattggacgtaagtaaatttattcggcgattcaagtaaaaagttggcatttatacttaactaatggatgtatgatatcacatgtgtttgaacatattaacttgatgtaaacaccgaatacgagtaaatacttatcaatgaaactgaccatactctttccacgagccgagcggtgtgccatttatgtccatctgccgacagccctggcacactgcggtgcaagaaacatgcaaggcccaagggcacatgaaaaaggagaggaaactcacatgctatgaaataaattcacatttgacaactccataaacagaaaatgcaaaaaaaaatgtctttatgtaccgaatttacttatttttgcacagatattatcctttcattaaaaggaacaaactaaagaacactcgatattaagaacaccaaactacaaataaacaaccataggaacatgataggtttgcgaacaataaatgcccattttcaagtacaacaaccccctccttgtgaagcagctccgtctcttccacctgaaggcccgggaatgcctccatatccaacttttattccatacgactgcaaaagaaaaaagggaagcgtaagtttcggaaaccatctattattacgtaaatgataattatacatgttggaaatcaattatcactgagatatacaagtatttactgaaatatttgcgacgtaagtttttgttttcttggttctcaaaacaataattaggacactaggattgttccaaacagaactgaaaccgataacccgaataaaaaaagttattggtttatcaatattgggttattagtttagcggttttgataacgatttagattttttttgttatcgggttatcggttcttaacggtttgaagttttttcttaacgggttaatcgataacccgatagtaaattaaataattatatttataccattttgtatataaagtctttgacttagagtttggtttcccactttcagttttggttgtctcagacacttctcgagggtaccacattgattgagttttatatgcaatatagtaataataataatatttcttataaagcaagaggtcaatttttttttattataccaacgtcactaaattatatttgataatgtaaaacaattcaattttacttaaatatcatgaaataaactatttttataaccaaattactaaatttttccaataaaaaaaagtcattaagaagacataaaataaatttgagtaaaaagagtgaagtcgactgactttttttttttatcataagaaaataaattattaactttaacctaataaaacactaatataatttcatggaatctaatacttacctcttagaaataagaaaaagtgtttctaatagaccctcaatttacattaaatattttcaatcaaatttaaataacaaatatcaatatgaggtcaataacaatatcaaaataatatgaaaaaagagcaatacataatataagaaagaagatttaagtgcgattatcaaggtagtattatatcctaatttgctaatatttaaactcttatatttaaggtcatgttcatgataaacttgaaatgcgctatattagagcatatattaaaataaaaaaatacctaaaataaaattaagttatttttagtatatatttttttacatgacctacatttttctgggtttttctaaaggagcgtgtaagtgtcgacctcattctcctaattttccccaccacataaaaattaaaaaggaaaggtagcttttgcgtgttgttttggtacactacacctcattattacacgtgtcctcatataattggttaaccctatgaggcggtttcgtctagagtcggccatgccatctataaaatgaagctttctgcacctcatttttttcatcttctatctgatttctattataatttctctcaattgccttcaaatttctctttaaggttagaaatcttctctatttttggtttttgtctgtttagattctcgaattagctaatcaggtgctgttatagcccttaattttgagttttttttcggttgttttgatggaaaaggcctaaaatttgagtttttttacgttggtttgatggaaaaggcctacaattggagttttccccgttgttttgatgaaaaagcccctagtttgagattttttttctgtcgattcgattctaaaggtttaaaattagagtttttacatttgtttgatgaaaaaggccttaaatttgagtttttccggttgatttgatgaaaaagccctagaatttgtgttttttcgtcggtttgattctgaaggcctaaaatttgagtttctccggctgttttgatgaaaaagccctaaatttgagtttctccggctgttttgatgaaaaagccctaaatttgagttttttccccgtgttttagattgtttggttttaattctcgaatcagctaatcagggagtgtgaaaagccctaaaatttgagtttttttcgttgttctgattgttgtttttatgaatttgcagatgcagatctttgtgaaaactctcaccggaaagactatcaccctagaggtggaaagttctgatacaatcgacaacgttaaggctaagatccaggataaggaaggaattcccccggatcagcaaaggcttatcttcgccggaaagcagttggaggacggacgtactctagctgattacaacatccagaaggagtctaccctccatttggtgctccgtctacgtggaggtggatccatgaagcggtacaacttgcatacggcataggagaaactgaaatgccaactgtgatgaatgggcttatggtttggtgcattgaaaatggaacctcgccaaacatcaacggagtttgggttatgatggatggagatgaacaagtcgaatacccactgaaaccaatcgttgagaatgcaaaaccaacacttaggcaaatcatggcacatttctcagatgttgcagaagcgtatatagaaatgcgcaacaaaaaggaaccatatatgccacgatatggtttagttcgtaatctgcgcgatggaagtttggctcgctatgcttttgacttttatgaggtcacatcacgaacaccagtgagggctagggaagcgcacattcaaatgaaggccgcagcattgaaatcagcccaacctcgacttttcgggttggacggtggcatcagtacacaagaggagaacacagagaggcacaccaccgaggatgtttctccaagtatgcatactctacttggagtcaagaacatgtgtaacttttactcatctcctccaattatttctgatttcatgcatgtttccctacattctattatgaatcgtgttatggtgtataaacgttgtttcatatctcatctcatctattctgattttgattctcttgcctactgaatttgaccctactgtaatcggtgataaatgtgaatgcttcctcttcttcttcttcttctcagaaatcaatttctgttttgtttttgttcatctgtagacatgttcttgactccaagtagagtatgcatacttggagaaacatcctcggtggtgtgcctctctgtgttctcctcttgtgtactgatgccaccgtccaacccgaaaagtcgaggttgggctgatttcaatgctgcggccttcatttgaatgtgcgcttccctagccctcactggtgttcgtgatgtgacctcataaaagtcaaaagcatagcgagccaaacttccatcgcgcagattacgaactaaaccatatcgtggcatatatggttcctttttgttgcgcatttctatatacgcttctgcaacatctgagaaatgtgccatgatttgcctaagtgttggttttgcattctcaacgattggtttcagtgggtattcgacttgttcatctccatccatcataacccaaactccgttgatgtttggcgaggttccattttcaatgcaccaaaccataagcccattcatcacagttggcatttcagtttctcctatgccgtatgcaagttgtaccgcttcatactagtttttaatgtttagcaaatgtcctatcagttttctctttttgtcgaacggtaatttagagttttttttgctatatggattttcgtttttgatgtatgtgacaaccctcgggattgttgatttatttcaaaactaagagtttttgcttattgttctcgtctattttggatatcaatcttagttttatatcttttctagttctctacgtgttaaatgttcaacacactagcaatttggctgcagcgtatggattatggaactatcaagtctgtgggatcgataaatatgcttctcaggaatttgagattttacagtctttatgctcattgggttgagtataatatagtaaaaaaataggagctcatcgttatgctataaatttcatatatttagttgggagtaggctttataccgagttggactacggtcagtcactttcaagtcctagaactacgtgcccctgtaggttataagtctcctctgtgggcatcaatttagtgatcatgccagtcatgcctctatacctctgacaggatatatggtactgtaaac

SEQ ID NO: 3 - DNA, 33302 bp, synthetic construct
  misc_feature 1..33302: note = pSIM4617 T-DNA
  source 1..33302: mol_type = other DNA
tggcaggatatattgtggtgtaaacaaattgacgcttagacaacttaataacacattgcggacgtttttaatgtactgaattaaatgtttagttcctcgttttctctcgttggacgcaaagagggaactaaacacttaattacgcgttttataacggtcctaaggtagcgaaaaatagggataacagggtaatcatcctcgagcacattgattgagttttatatgcaatatagtaataataataatatttcttataaagcaagaggtcaatttttttttattataccaacgtcactaaattatatttgataatgtaaaacaattcaattttacttaaatatcatgaaataaactatttttataaccaaattactaaatttttccaataaaaaaaagtcattaagaagacataaaataaatttgagtaaaaagagtgaagtcgactgacttttttttttttatcataagaaaataaattattaactttaacctaataaaacactaatataatttcatggaatctaatacttacctcttagaaataagaaaaagtgtttctaatagaccctcaatttacattaaatattttcaatcaaatttaaataacaaatatcaatatgaggtcaataacaatatcaaaataatatgaaaaaagagcaatacataatataagaaagaagatttaagtgcgattatcaaggtagtattatatcctaatttgctaatatttaaactcttatatttaaggtcatgttcatgataaacttgaaatgcgctatattagagcatatattaaaataaaaaaatacctaaaataaaattaagttatttttagtatatatttttttacatgacctacatttttctgggtttttctaaaggagcgtgtaagtgtcgacctcattctcctaattttccccaccacataaaaattaaaaaggaaaggtagcttttgcgtgttgttttggtacactacacctcattattacacgtgtcctcatataattggttaaccctatgaggcggtttcgtctagagtcggccatgccatctataaaatgaagctttctgcacctcatttttttcatcttctatctgatttctattataatttctctcaattgccttcaaatttctctttaaggttagaaatcttctctatttttggtttttgtctgtttagattctcgaattagctaatcaggtgctgttatagcccttaattttgagttttttttcggttgttttgatggaaaaggcctaaaatttgagtttttttacgttggtttgatggaaaaggcctacaattggagttttccccgttgttttgatgaaaaagcccctagtttgagattttttttctgtcgattcgattctaaaggtttaaaattagagtttttacatttgtttgatgaaaaaggccttaaatttgagtttttccggttgatttgatgaaaaagccctagaatttgtgttttttcgtcggtttgattctgaaggcctaaaatttgagtttctccggctgttttgatgaaaaagccctaaatttgagtttctccggctgttttgatgaaaaagccctaaatttgagttttttccccgtgttttagattgtttggttttaattctcgaatcagctaatcagggagtgtgaaaagccctaaaatttgagtttttttcgttgttctgattgttgtttttatgaatttgcagatgcagatctttgtgaaaactctcaccggaaagactatcaccctagaggtggaaagttctgatacaatcgacaacgttaaggctaagatccaggataaggaaggaattcccccggatcagcaaaggcttatcttcgccggaaagcagttggaggacggacgtactctagctgattacaacatccagaaggagtctaccctccatttggtgctccgtctacgtggaggtggatccatggcggctgctgcctcaccatctccatgtttctccaaaaccctacctccatcttcctccaaatcttccaccattcttcctagatctaccttccctttccacaatcaccctcaaaaagcctcaccccttcatctcacccacacccatcatcatcgtcgtggtttcgccgtttccaatgtcgtcatatccactaccacccataacgacgtttctgaacctgaaacattcgtttcccgtttcgcccctgacgaacccagaaagggttgtgatgttcttgtggaggcacttgaaagggagggggttacggatgtatttgcgtacccaggaggtgcttctatggagattcatcaggctttgacacgttcgaatattattcgtaatgtgctgccacgtcatgagcaaggtggtgtgtttgctgcagagggttacgcacgggcgactgggttccctggtgtttgcattgctacctctggtccgggagctacgaatcttgttagtggtcttgcggatgctttgttggatagtattccgattgttgctattacgggtcaagtgccgaggaggatgattggtactgatgcgtttcaggaaacgcctattgttgaggtaacgagatctattacgaagcataattatcttgttatggatgtagaggatattcctagggttgttcgtgaagcgttttttctagcgaaatcgggacggcctgggccggttttgattgatgtacctaaggatattcagcaacaattggtgatacctaattgggatcagccaatgaggttgcctggttacatgtctagattacctaaattgcctaatgagatgcttttggaacaaattattaggctgatttcggagtcgaagaagcctgttttgtatgtgggtggtgggtgtttgcaatcaagtgaggagctgagacgatttgtggagcttacgggtattcctgtggcgagtactttgatgggtcttggagcttttccaactggggatgagctttcccttcaaatgttgggtatgcatgggactgtgtatgctaattatgctgtggatggtagtgatttgttgcttgcatttggggtgaggtttgatgatcgagttactggtaaattggaagcttttgctagccgagcgaaaattgtccacattgatattgattcggctgagattggaaagaacaagcaacctcatgtttccatttgtgcagatatcaagttggcattacagggtttgaattccatattggagggtaaagaaggtaagctgaagttggacttttctgcttggagacaggagttaacggaacagaaggtgaagtacccattgagttttaagacttttggtgaagccatccctccacaatatgctattcaggttcttgatgagttaactaacggaaatgccattattagtactggtgtggggcaacaccagatgtgggctgcccaatactataagtacaaaaagccacaccaatggttgacatctggtggattaggagcaatgggatttggtttgcctgctgcaataggtgcggctgttggaagaccgggtgagattgtggttgacattgatggtgacgggagttttatcatgaatgtgcaggagttagcaacaattaaggtggagaatctcccagttaagattatgttgctgaataatcaacacttgggaatggtggttcaactggaggatcgattctataaggctaacagagcacacacttacttgggtgatcctgctaatgaggaagagatcttccctaatatgttgaaattcgcagaggcttgtggcgtacctgctgcaagagtgtcacacagggatgatcttagagctgccattcaaaagatgttagacactcctgggccatacttgttggatgtgattgtacctcatcaggagcacgttctacctatgattcccattggcggtgctttcaaagatgtgatcacagagggtgatgggagacgttcatattgaactagtttttaatgtttagcaaatgtcctatcagttttctctttttgtcgaacggtaatttagagttttttttgctatatggattttcgtttttgatgtatgtgacaaccctcgggattgttgatttatttcaaaactaagagtttttgcttattgttctcgtctattttggatatcaatcttagttttatatcttttctagttctctacgtgttaaatgttcaacacactagcaatttggctgcagcgtatggattatggaactatcaagtctgtgggatcgataaatatgcttctcaggaatttgagattttacagtctttatgctcattgggttgagtataatatagtaaaaaaataggaattagcttatcgataccgtcgacctcgatcgagggggggcccatggtaccagttatacaccctacattctactcgagtcattatgatgatgtctcacgaccaaatcaaatcaaagttaaataaatatcgaaccgaacgcccactctgtatgagtatggcaaaagattttgagagaatcaagttgcataaaagcctaattttcatggaacatacaaattgagtctcataatagcccaaactcacagccatgaacccaaattgggtaaagttttgcaagacgttcatcaaacagttaggaaacataaaatggcgctagatatataataaatttttttaacatatggtgtgattgatagttatatactaaagatgtttgcttagttacgtaattttttcaaaaaaaaaaggtacattatcaatcatcagtcacaaaatattaaaagttactgtttgttttttaaattccatgtcgaatttaattgaatgacacttaaattgggacgaacggtgtaatttcttttgactattctactagtatctatccacagcacgtgttgttcctttcttctttcgtttttcatttacttgacattattaggagacttggccctgaactccaactattctaagctgacctttcttttcctttaccaattatcttcttctttctaatttcgttttacgcgtagtactgcctgaattttctgactttcaacgtttgttattcatgcttgaaaacgaaataccagctaacaaaagatgaattattgtgtttacaagacttgggccgttgactcttactttcccttcctcatcctcacatttagaaaaaagaaatttaacgaaaaattaaaggagatggctgaaattcttctcacagcagtcatcaataaatcaatagaaatagctggaaatgtactctttcaagaaggtacgcgtttatattggttgaaagaggacatcgattggctccagagagaaatgagacacattcgatcatatgtagacaatgcaaaggcaaaggaagttggaggcgattcaagggtgaaaaacttattaaaagatattcaacaactggcaggtgatgtggaggatctattagatgagtttcttccaaaaattcaacaatccaataagttcatttgttgccttaagacggtttcttttgccgatgagtttgctatggagattgagaagataaaaagaagagttgctgatattgaccgtgtaaggacaacttacagcatcacagatacaagtaacaataatgatgattgcattccattggaccggagaagattgttccttcatgctgatgaaacagaggtcatcggtctggaagatgacttcaatacactacaagccaaattacttgatcatgatttgccttatggagttgtttcaatagttggcatgcccggtttgggaaaaacaactcttgccaagaaactttataggcatgtctgtcatcaatttgagtgttcgggactggtctatgtttcacaacagccaagggcgggagaaatcttacatgacatagccaaacaagttggactgacggaagaggaaaggaaagaaaacttggagaacaacctacgatcactcttgaaaataaaaaggtatgttattctcttagatgacatttgggatgttgaaatttgggatgatctaaaacttgtccttcctgaatgtgattcaaaaattggcagtaggataattataacctctcgaaatagtaatgtaggcagatacataggaggggatttctcaatccacgtgttgcaacccctagattcagagaaaagctttgaactctttaccaagaaaatctttaattttgttaatgataattgggccaatgcttcaccagacttggtaaatattggtagatgtatagttgagagatgtggaggtataccgctagcaattgtggtgactgcaggcatgttaagggcaagaggaagaacagaacatgcatggaacagagtacttgagagtatggctcataaaattcaagatggatgtggtaaggtattggctctgagttacaatgatttgcccattgcattaaggccatgtttcttgtactttggtctttaccccgaggaccatgaaattcgtgcttttgatttgacaaatatgtggattgctgagaagctgatagttgtaaatactggcaatgggcgagaggctgaaagtttggcggatgatgtcctaaatgatttggtttcaagaaacttgattcaagttgccaaaaggacatatgatggaagaatttcaagttgtcgcatacatgacttgttacatagtttgtgtgtggacttggctaaggaaagtaacttctttcacacggagcacaatgcatttggtgatcctagcaatgttgctagggtgcgaaggattacattctactctgatgataatgccatgaatgagttcttccatttaaatcctaagcctatgaagcttcgttcacttttctgtttcacaaaagaccgttgcatattttctcaaatggctcatcttaacttcaaattattgcaagtgttggttgtagtcatgtctcaaaagggttatcagcatgttactttccccaaaaaaattgggaacatgagttgcctacgttatgtgcgattggagggggcaattagagtaaaattgccaaatagtattgtcaagctcaaatgtctagagaccctggatatatttcatagctctagtaaacttccttttggtgtttgggagtctaaaatattgagacatctttgttacacagaagaatgttactgtgtctcttttgcaagtccattttgccgaatcatgcctcctaataatctacaaactttgatgtgggtggatgataaattttgtgaaccaagattgttgcaccgattgataaatttaagaacattgtgtataatggatgtatccggttctaccattaagatattatcagcattgagccctgtgcctagagcgttggaggttctgaagctcagatttttcaagaacacgagtgagcaaataaacttgtcgtcccatccaaatattgtcgagttgggtttggttggtttctcagcaatgctcttgaacattgaagcattccctccaaatcttgtcaagcttaatcttgtcggcttgatggtagacggtcatctattggcagtgcttaagaaattgcccaaattaaggatacttatattgctttggtgcagacatgatgcagaaaaaatggatctctctggtgatagctttccgcaacttgaagttttgtatattgaggatgcacaagggttgtctgaagtaacgtgcatggatgatatgagtatgcctaaattgaaaaagctatttcttgtacaaggcccaaacatttccccaattagtctcagggtctcggaacggcttgcaaagttgagaatatcacaggtactataaataattatttacgtttaatatccatgatttttttaaatttgtatttagttcatcaactaaatattccatgtctaataaattgcagggatgcctttgaaaatgattctgtgttggagagaatcttctgatgcctgttggtattataatactaataataagagaaaaagtttgattactgtttcaagttaattgcttgtgatttgtaaaaacaaattacttttatatttctctttgttttattttatgtttatttatctttaattaatggagtaataaaataaaaatcttattttcaatagaaaaaagtagaccttatttgtggtgcatgtatggtatctttttgaaatttttgatatatttgctctttgattcgaatttcttgcttatatgatgatttgcataaatataaaatattatacaaatacctatgggttggaaaatatagaaatatgccaatcaaatgtatacaaaaatcattaatagatagaatcgtaaaagatatacaaatgagaaatgcttgactaagaagcttcgtgcaacctctcacactgagcacaatgcatttggtgatctcggcactattgctgttacttgtaagactacgttccccaataagtctttccaaacggcttgcaaagctgagaatatgaaaatctcataggttagtttgctgcgttaattatttacatttaatatgctcgataaggtgattttaaaaaaatttgtactagttaattcatgaactaaatatttcatttaatactccataattctgaatatggaaaataaataatatttaataacaagaataaaatgataaattattcattgattttataaattggataaatattattaaatattcttaaataatataatgaacaagtgaagatgaacggagggagtatgaagcctcttttcaaaggggcccaaaaacgttaaatctagaaggcctcccgggaaaggatccaaagaattcaaagtcgacaaaatcgataaagagctcaaacctgcaggaaaggtaccaaaccatggaaaggcgcgccgtttaaacaaactgataattgccaacacaattaacatctcaattaaggtaactaaaaaactccttatacctcgaggaaacgccggcgaaagcggccgcaaacgtcgacgaaagcgatcgcaaattaattaaaaacgcgcgcgaaacctcagcaaagacgtcaaaaccggtaaacctaggaaaactagtaaaaagcttagagctagcaaacaattgaaagggcccgtccatatgtggaagctactctctttgtccaaaaatagttgtccactattgacttacacacaccttaagaaataataaataataggggtactattactacactatcctttgaatattataaatttaatgctctgggaaaatgtattagatattgaatactccctccgtttcaaaaagaatagtctactttgacttggcaaagagttttaaaaaataaagaaaatttttcaatcttgtagttctaaattaatgttatgtcaaatgtaccaaaatatcttttaatcttgtagccttgaacatgccacgtggaaagttgaaattaaagtgttgatcaaaaaggaaagaggtcactattttctaaacggaggggtggtatttaattgtaggggtaaaatagacacaaaaggataaattatcttttgattttctaaattggacaagtattgttggacaactatttttcgtatagtgggcaagtaaagatggacgggggaggtattaccaagtctaaaaatgggaagctatggccaagtctaaactgatactccatgtgttcctttttatttgtcacgtttttttttttttgagaaatcaaactacatgaattttgaccaatattttaagatctactttttcaaatagttctcaaacatctaaattttaaataataaattattctaattcaaattagcttcaaaaattagtcaaattgactttcagaaagtcaattgtgacgattaaaaaagaatggagagagtaagtttttttaaaatcttattctccattatttacatgacgagtcttggagaattttcactttgttaaggattcgattcttcatatcgtaatcccttcccctattcttcctttcacctgcccctttttgaaaattaaatatgacccagaggctgcttttttatatattgcttcacataccccatttttttattttttattttgaaaaacggaaaaagacatgacgagccccactagtctttcattatttctcattttttgtacacattgaatttctttggaatttaataaaggtagtacttagatttgaccaatccaaactatgtgcctttggcgtgttgctttttttaaaaaaattattttttgaaacttttgataataagttgtcacatgtgtagcatttttgactttgatcttcaaggcttattgagggcaagtggggtcgaaaattcaatatcaccgatttataattggatggggatttttatttacatctggcattagtcgattttgaaaagaatgtctctttcgtaatttgagaactcgttaatcacaatattgcacatcaacagataacatgtttaagatcacaaaattaaaaaacaatttgatatattattacacatatctttagtttaacactataagattcaaaaatcttctttggtttctgaaacttcattgtcgaatcaaataaaaacgaaaaagttgaaatagagagagaaaaggtagtttccttgaaatttctctagatttggagcaatccaatgtttttctgtatcaaaattttacatattgatggtgagtactgtcaataatgtagtactccctctgtcccaatttatatgactcactttcctttttggtcagtcccaaaaagaatgacacatttctacatttagtaacaatttatttttaaaatgacttttttactcttaatgaaatgatttctagccacacaaatatctaccactcattttagaccacaaatttcaaaagtcttcctttctttgttaaactccgtgccgagtcaaactatgtcatataaattgggacggagggagtatgatatttcttctcttttgagtaatatttaatatcttcaattaaatcctatccactcctcatctattcttgttcatcatttttgagaattaatttgatggcagcttatagtgctgtaatttctcttcttcaaacacttattgatcaacaaaatatttcagaactctttcatggtcacactgcccaaacgctcgattctcttcacactacagctgaatattttcaacatgtccttgaaaatattacaagatttgactctgaaaagatcaaatctttggaggaaaaaataagagttgttgttagctatgcagaagatgttgttgcaatgaaaatttctcaaatcatcataggctcaagctggacatttggaattttacaacaccaggatttactaccacttgttgaaaaaatggacacaacaaagaaacaagtgatggacattctttctcatgatgatgatcaaattcttgaattaactgcaggggattccttgattggcacttcttctacaacttatccaatgttggaagatgatatcgtgcagggaattgatgatgacttggagatcatagttaaaagattgacaggaccaccacgggatttagacgttgtcacaataacaggtatgggtggcattggtaaaacaacactcgctagaaaggcttatgatcatctcacaatcaggtatcactttgacattcttgtttggattacaatatctcaagaatttcgatgtagaaatgtattgttagaagctttacattgcatttcaaagtcaacagatattgtgaacacaaaagattatgataagaaggatgacaatgagttagctgacatagtacagaaaaaactaaagggtccaagatacctagttgttgttgatgatatttggagtagagatgtttgggatagtataagaggaatatttcctaattacaacaacgggagtcgaatcttattgactactagggaaaacgaggtagcaatgtatgcaaatacttgtagccctcatgagatgagtcttttgagtttagaaaacggttggaggttactttgtgataaggtgtttggaccaaaacatgatcatcctcctgaattggaagaaattggtaaggaaatagttgaaaaatgccaaggactacccttaacaatttcagtgattgcgggacatgtttctaaaatgcccaggacattagaatgttggaaggatgtcgcccgaaccttaagtgaaatcatctctagtcatccagataattgcttaggagtgctcggtttgagttaccatcacttgcctaatcacctcaaaccttgctttctttctatgagtagtttcccagaagattttcaggttgaaactcggagattgatctacttatggatcgcagaaggtttcataaggacttgcgaaaatggtaaaagtttggaggaagttgcagtagattatttggaggaccttattagcaggaacttgatacaagctagaaaaaggagattcaatggtgagataaaagcatgtggaatacatgatctactgcgtgagttctgtttgatagaagctgaaataacgaagcatatgcatgttgagagaacttacccaactcttccaacacaaaagaataatgttcgtcgcttcagttttcaaacaaaattttattcagttgatgattgtaataagctattaccacctgttgccagatctatctacttcttttctcagttggatctacctgttgtaccctataagaggtatctcaggtgttgtttgcccatccaccgtgatgatcgtataatacatgatttttactctcgtttcaaccttctcagggtattggtcatctccaagacaaatgaatacttcgagtcatttccacttgtgattacaaagttgtttcatttgagatatctccaagttcgatttcttggagacattcctgaatcaatctcaaaccttcaaaatttgcaaactctaatttgtagtggtggtactttacctgggaagatatggatgatgaagaacttgaggtatataagtataataggcaacaaagtcacttatttaccaagtcctagaacagaaagtcttgtgaatctagaggagttttctgttctttgttacagaagttgtacaaaagaagtcatttctggcattcccaatctaaagagattgaccattgatgtactttctagcattaacaactattttcccaatggactaatagatatgtccagcttgacaaaactcgaagcattcaagtgtaataggtgtttatattcgaatttcaatagttctgttattccaacatcacttaaggattttgtttttccaacatcacttaagaggttgagtttaaactattatgctagtcattttttttgggaagaaatatcatcaactattatcatgttgcctaatcttgaagagctgaagcttaaagattgtcgatccgatgaatatgatgaatggagtttgagtgataaagacaaattcaaaagcttgaagttgttggtactaaccgacattttttttgatcgttgggaagctaccagtgataacttcccaaatctaaaacgccttgttctgaacaagtgcgacttagaaattccatcagattttggggaaatttgtactttggaatcaattgagttacatgattgcagcactagtgctgaggattctgcacgagagattgaacaagaacaagaggagatgggaaataatatccttaaggtctacatacatggcagtcgcagtaagttctaaacagttattcgattctctcatctttgttaaatcttacctccttgaagttatgatgatgacttcgattttttgtcttgcataggcaggaaaatggaagcatatttcgaggaatcatctaatgccatcttccaaatgcagaaccttttagattaatcttttatgttctgattgtttgttaaattcatgttttaggaataaacacgtttaatacatgtttcgctctttctttactatgtaaacagtaactattgatgtattgttatatctattttgtgttatgtgatagttcgaaatgaataatgtgttggggttttggtctttttcccttcctatgtggataaataaaagcccaattggtctagcccacttatgcccataagcccaatttatggagcatacataagactttctttaagtcttattttcatatatcacacataatttttacaagagagataagcatacatattagagagaaaagagagaaagtgcagattttcgcacaaaccctagcagccaatttcaaatcgcgattcccgcttcgtttcttatctgattaagctgatttttggacagcacgttcctctcaacttaatctttgattgagaactgacagaggtcgatttggagtcccgtagctccagatttttgctcgtgaacagcagctgctatttggtgattttgctccttttacttctctagttctttggtgctatcttgtagttgttgttgctcgtttttggcacttgttttggtgaccattttggagaggatcccccggggatctagaatttaagctttatggatgtaatattatttttaaagttatatgttcacatctattatttgttataattttcatgaaaatttatatatatatattttgcatgtatgttgcagtgaatatcttagattagtgtattttattatcagggtttttgtaatatttcatttttttatgtgggcacctaacatagctatagagagctttctttcaagttctcacgatcggacgtataaaaagtacctacataaaactttactcaggagaaagaagaggaaagcagtagaaaaagaatacaagaaactgaagatcaaaagcagcataaaaagtcaaaacctctcactacaaatatctttcctatttttatgcctcatatattattttatctaccctccccatcatgtttcattccttcaaaccatattatataggaaactcaattttgtaaatatgtacgtctacctggctaatattcatattacaagttgactaagacttgtggagattgtcagcttgtgaatgaagatgatgtctgatgcaactttggtgttgtggtttatgagaattgacgggagcctacgagcaaccataaagttgtctccatgtaacctatagatcatgggtttgagcggtgaaagaagccactaatttttctacagtatcctcctgggatgcgatccctccattaatcctaccaacgttggatgttttgtgcatgaggctgctttgtttacgggacattcctgatgtttcttgtttttcatagttcccctttagataaagtaaacaagagagtttgaagatgaacttaccttttgacagtctttagtttagcaatattagaagcccttgtttgagaaataatggcaaagagagttgtttgctaaatgaagtatagttgaatgataaattttatatacctagaaacaaggatcaaattcccacttcatacttccttttttctcctttcattagtggtgcattcatgctctagaaattctagattcgcttctgaggtgtaaatttgtattagcagtaaaaatctcaaagaggaatgtacaatctcttagagtcgagatacacctacacatcttgcttttactgttctatcgggagttacttcatgtattgcttgacataaaactgattgtggattcgtcttggtagatatccaggtaatacatcctgtctgctctagccacactaaccatatgcatcaggcttgaaatcgccctccgggatagtatgtgcttgtcaacttagagttggaaccaccatctgctgactgctcaaacttcacctcattaagatatttttcgagtttgtcatcaatcctatactttatgaatttaaagttgccacctgcagaaaagaagtcacaaatagaagaatattagtgattaaaatgagagtctattaaaaattgaaaaagaattaactaggatgaagaatgttgatattcatgacaaattcaatacttctgtagcttgtggctctaacacatttcagattctcaaaagtgtcttcctcccccatgttccattcttctccctgaatgattgtataataaaggattcctctccaacctccagtcttgctattgttgatagtgcatcggatgtcagaggaaagtcatacaacaacaattttttcaaattcgagattagagactgcccctttggcgatgaggctctgttggctaatgctgcaaagctggagacaatgcgatccctttggatgtctaattgttcagtaagttttgaagcatgtaagctgttggtccagaagttgccagggcttaatgttgaagttatagacgagaggggtcatccagatacgagaccagaaagttgccctgttgagaaactttacatatacaggacagtgtcaggaaggaggttcgacactcggattattgatgaagatgcagcattgacttgttcttctgttgaaatggtgctgtcaatgtacttgtggtcatgaattgtcgtaattctttttgaagattaatgtgaacctttcattcactttgagtgctggaaatcccatttgttgaacgtttagaagaaggtatttcagtgttctactacgagagttaatttgataaacaaacacttattttcgttcagagtgggatttcagcctacgttggttgaatgttcgagaaaaggaagtcatgagtgcttataagctctctacaacctcttcacttcctggcctagcttttgagatgaaagttcgttgggtttgtgataaagtagtatagatgtcattttaaatcagtaccttgattgaggttgaacgatagtgctctgagcatatgagacttcagaccaagttggacagccaaaggcaaatccacattgtatttataaaaactataatttttgttaaaaacttctgagttttctaaaagaaactcagaaaagtcaaataaaaatgtttcataacaactgtaacagaattttcttaaccatatacaatctacccagcaaagtgcaatctacttttcaaccataatgctcaaaaactacttaaataacgggatatccttctggccaaggatctgaagctcgtcccctcccctcatatcttcagcatattccttaatcttgagagcggaattttcaagttgagggctccttacaagttcgataattttcaaggaataaatatccccaaaactagacggaatctcctcaagattatgacagtccgacagttctaatttctcaagcgtgggaaaagattcctctccaacctcccacttggaaagaatcacttgactcaacatcaaacatttgagattctcaaaggtgtcttcttctcccatgttccattcttccccatggatgattgtacgataaaggtacaactcttcaaggttcagcagtctcgctattgttgatagtgaatcggatgtcagaggaaattcatgcaattgcaatcttttcaaactcgaaggaaagtgaaaatcccatggccgatttatggctgcagaggacccactgtcatttgtgtttgatctttcaaaatctacagtgagtttttctagttcagttaggaaatccaatttcgggaaccaatattgctctgttgaataatcccatgactccttgagtttgaaatgaagcacttgaagattgggaagccttttgaaaatatcctctgtatctttccaataggaaagcacgagttcccctaatgctgtcaagttctctaactttgtgtcctctgctatcagtattgattcatctgcatccatatcaaagaaagaacaagcagtcgtgaacagcacttgcaactttacaagatcccaaattctcggtaatagtatcaaggttgattctttgttatccacaaacaagatttctagattccagaggtttgagaaagacaaaggcagagatttaacttctgtcccaatgcttaagtacctcaaatgattcaacatgcatatttcattcagcaaagaatctttaaccatgataaaagaggattccaggtgcaaggttctaagaagcctcaagtgtcttagatgaaatgtatcagaaagatggtcgtccagctcatctccatttatggtcaaagaatagaggtgtttaccggaatgccttttcttatttgaaccgaacaggacaaaattaagcccaaagtgctcttcatcatcatcataatcaatgctaatttgacgtggcaacaaatctgatggagcacttgaacttatccgatcacacaacttttcctttcttgcttttatcaaacaaaagtcatgcacaagatcatgaagttggcaagtagggtaatcacctatctcattgaaacaaattaccaagctactggaaattaaatcatccaaataaattttcaccacttcttccagactcttcatatctgtcttttccacaaatccttcaaaaccccaaataagtttcaactcatggattgtcgttacccagtccttcggcgcacttgcaaagtacagcaagcatggcttcaggtgatcaggtaagtggtcataacttatttctataactttcatcacttccacttcattcttcaaaataaaggaatgcaaattatttacaacttcaagccacacactctttttcttttccctcccagcaatgattccagcaatcagatccaccaccaaaggaagccctttacaattttcggctatttctttaccaacatccaatagttcatcagggcaactctcgtttccaaatgcccttttctctaataactcccaactttcttctgatcttagcaatcgaaggttaagaggatcagtgtagagctttccatgcaaagcaactttcttttctcgagttgtcaaaataattctacttcctttcataccatcaggaaaaggtcttgttagctcatcccatgtattagtatcccacacgtcatctaagacaataagatacctctttccaaacaattgtttccgtagtttatcagcaacatcaatattctcactcaattttgaatttgagtcactaacttgattgaaaattttatccaacaacttcttctcgtcatatacttggtcgaccgtgcaccatgcacgaaggtcgaaatggctagaaactgatttatcattgtatactttgtacgccaaagtagttttacctaaacccggcataccaatgatcgaaatgacatctagatctgccggtccactggtgagctttctaagtatcaagtttgtctcctcaccaaaacctacaattattttatcagttgtcaatgacttgctctcaactggtttcttgggagagttcacaacgatgagacctctgttcttggaaatgttctcatgtaaatcagagacctcttctttgataagcatcatcttctttctggtaatgggaagtgagaaaataagatgtaagagaccattatctcgaacaataattgaatctatgacatcttttgcctcatatgccacatctagaacacgttcccagagatctttatacaatccttgctcaatattcgcgaaaaaagatcttatgaattccaagtcttctttcaccagcccaatttgttcctttatcaaagcaattgaataagcattggaatccagcaaatcatctaagtgtctctgtagcagatgcatgaagagaggtccatcactcatggggaagcaatattgagatgaatccgggactttcagataaacatgtttgagatcttccttaaggagttcaatattttccagaaactttagggttgcacaatttgtttcgtcagtactctctttaatccttaatttctcttccaagtcgcgtacaagagttgatacctccctggtaagtgctacaacacgagccaagagatcaaaaagtttgtcatgatgaataaagtccttgggcggcatatcagaaagaataatcaataggaattccatcatgacatgaatgtttcgagcccctgaagtgttaggggtaataacagttatcatatgctcttgtagatgaatcagatattctctgagaatgtccggagaggtttccaggagcttcttaatgaagcgtccaatttctgttgaagttgaagctttcaaagttttataacatatgtgcataacctccaattcagttggaacaatcttcaagagtagatgtgctagcttgaagagttgagggtctttatcattctgatcatcctcatctagctcggagagttgagagtcttcatcagcctgatcctcccaaaggaagcgtcctactctctcagccatcagttgaaacagagataagacattctcaaccatctcatgcttaatgcaacaattcactatcaatccatggaaatctcttatgttgccacatacattctgaagaacctcatattgagtcactccaggaaacatcttttcagcacgatgcttggatagatgagagagattcaagaggaggaagcccaattgctcatccatcatggtggcatctgatttagaacgatgatacaagcttatacagtcatccatattaccggcgaggctagtaaggacatatttacacccgacgtctttgccatcatcatccaaaattggttgaagcagattctcaacctcttgtctttttctagtcattatatcttcaaacttctccaaatcggaataagaaagctggacatatgtacaaataaatgtcagcttcaatttcaggctttcaatcagatccacatcaacagccttttgatcttcttcattctttaatctctctaggaaatccagaacattggcagcatccttgcgaagagcagaaaatgactcctgccaaaataacacatatcaaatttttaaagccacatgatatctacaattcaatactttacagttcactctatcaaataacataccaatgagttgtttgcttcttcattatcttttcgtttttccatgattctacttttcctttccaagttctcaagacctgatgcataaaaagtacataaaactttatcaggagaaagaagatgaaatgataagaataatcaataatttcactattttcacgcgaacttaagaggacttaattagtggaagatgataaaggtacacttgcgatttgaattgaagaaaatgacttgtaaaaatggggttcatccagaaacgagaccaactggtaccgaacctttctatctctaatagcgtatagtaatatattttcattagtgaggggaatctttcgtcatcttagaaaaaatctaaaacatctgcttctgttcactccacactctccaaccccatccctctcacccggtaagaattcacccgcaccggatattaataccaaattaatacaacttatcatgggtaagtaataatttaatgaggtgaaagtatacattaactaaccacaattaagtgatttgttgtaaatcggtggtgtgagtaagtatttaacctctctctttctcctgcaatttatttccctttctctttgttagatccaactaacctactaaagttctttcatttttcttacatcacttctacttttccataactgtacttcaatagagatgagagtagcaaatattagatgagttgactaacctttttagttgatgaaactatatatcctctgcttgctttgttattagatgtatagagctcaaaattctggaaataaacagaggaataattaagaccaaatatgatgcatagtattgttccaaaaagtcaagaaagcaaaagcatgaatcaattacagtggggcctcctcacttgtctgccacaaattaggaaaaagataaagaaaatgagaaaatagtcaaatgtcccctaacctatactcaaattttcaattacagacttaaatttcacaagggtctttgttaattaccatcgattttcaagacctccaatgttaattggatgtgtttttatttttaatttattatttgatttattcatatttattcattattaacacttttactgtgtgtctttacaggtacacatgtttgcttctcattgaagctattcgaatgaagttcgaacaacattctttatgttaagcagttagttaaatgcactgcagtgcactaacagaaaatgtacacctatcagtctagttgttagagtcagttaaattaactgcttcagattagaatataaatagagatatatagattgtaatagtatagttcttctcattatttttttcattcattttggttcatgagttcaatatagaatttttctctcttcttctccttcaatcagtctttctaaaatagttaattctctccattgtttagttagaattcactgaaattacatggtatcagagcatttgaagcagatctagggttttatctcgggattttttgagcatagatcgagagaatctgcaatggcgattgaagaggaatcagtggatgaaattcatggtagaactggaggagctgtaccgaggggaggttcggtgattctagatcctctagagtcgacctcgagggggggcccaaaaacgttaaatctagaaggcctcccgggaaaggatccaaagaattcaaagtcgacaaaatcgataaagagctcaaacctgcaggaaaggtaccaaaccatggaaaggcgcgcctttataacggtcctaaggtagcgaaaaataggaataacagggtaattttacgcgtaaatgtttagttcctcgttttctctcgttggacgcaaagagggaactaaacacttaattacgcgttttataacggtcctaaggtagcgaaaaatagggataacagggtaatcatcctcgaggaaacgccggcgaaagcggccgcgaaccatgcatctcaatcttaatactaaaaaatgcaacaaaattctagtggagggaccagtaccagtacattagatattatcttttattactataataatattttaattaacacgagacataggaatgtcaagtggtagcggtaggagggagttggttcagttttttagatactaggagacagaaccggaggggcccattgcaaggcccaagttgaagtccagccgtgaatcaacaaagagagggcccataatactgtcgatgagcatttccctataatacagtgtccacagttgccttccgctaagggatagccacccgctattctcttgacacgtgtcactgaaacctgctacaaataaggcaggcacctcctcattctcacactcactcactcacacagctcaacaagtggtaacttttactcatctcctccaattatttctgatttcatgcatgtttccctacattctattatgaatcgtgttatggtgtataaacgttgtttcatatctcatctcatctattctgattttgattctcttgcctactgaatttgaccctactgtaatcggtgataaatgtgaatgcttcctcttcttcttcttcttctcagaaatcaatttctgttttgtttttgttcatctgtagcttggtagattcccctttttgtagaccacacatcacgactagtcattacacattcctcccggatcaacccgattccggccaccggaagtcccttaaaatcatctccggcattttcctctcctctttccttttgctttctgtagccttctttccgatcctcaacaaccagtcaccggacttgcagagtaactcccgttcgccggcgccgccgtcaagaggtgtttctcagggagtctccgataagacttttcgagatgtcgtcaatgctagtcacgtttcttatgcgtggtccaatgctatgcttagctggcaaagaactgcttaccattttcaacctcaaaaaaattggatgaacgatcctaatggtccattgtaccacaagggatggtatcatcttttttatcaatacaatccagattcagctatttggggaaatatcacatggggccatgccgtatccaaggacttgatccactggctctacttgccttttgccatggttcctgatcaatggtacgatattaacggtgtctggactgggtccgctgaattcctctaaagtccttgacaccaatgccgggtggaggaaccagaaccgggttgcctttgtacttgacccagtctagaaggagaggatcagataagttggtggggtacgcaagattttgcacttgtacataatcatcagtgtcaccggtataaagcatcatgatctgaccatcgggtaggatggtagcggacccagtccagacaccgtttatatcgtaccattgatcaggaaccatggcaaaaggcaagtagagccagtggatcaagtccttggatacggcatggccccatgtgatatttccccaaatagctgaatctggattgtattgataaaaaagatgataccatcccttgtggtacaatggaccattaggatcgttcatccaatttttttgaggttgaaaatggtaagcagttctttgccagctaagcatagcattggaccacgcataagaaacgtgactagcattgacgacatctcgaaaagtcttatcggagactccctgagaaacacctcttgacggcggcgccggcgaacgggagttactctgcaagtccggtgactggttgttgaggatcggaaagaaggctacagaaagcaaaaggaaagaggagaggaaaatgccggagatgattttaagggacttccggtggccggaatcgggttgatccgggaggaatgtggatccgttaacaagcttgttatcaactgatctctctactagagagtgtgtggtagagtgtgattgcacttgaattacagctgagatttttagttttataatactgatatcaagggtaactttatagacctgattttgtcggaatttacgagaggaccataggtatgattggtacacacgtcttgtgattgagggtattttggggatttaagcatttcttgtgattgaggctattttggggattgaaatatgacttttgtacttagcttaaaaaaaatatctaatgatgaaaaagaatatactattagaatcattatttttcgaaaaacagttactcaattttaatttgtttgaactacgttatttttcaaaaataaaattaaaagatattttgatatatttaacttatttttaatttaaaacaataaaaattcaataataaaaaaattcttatcaaattaaataaattaaactgaacaaagtattcaaaagattatttattagtgaacgtcatttcacttgtttgcacgagagttagatatgatcacatacgcttgagagaataaaaaaagagtggcggacgtgtataaaccgttgatgattaaatgtgattattagtgagaaaatgaattgtttaatcaagattttttgtatttaaataattggtatgatcacatgcgcttgagagaataaaaaaagagtgacggatgtgtatgaaccgttgatgattaaatgtgattattagtgagaaactgaattgtttaatcaagattttttgtatttaaataattggtatgatcacatgcgcttgagagaataaaaaaagagtgacagacgtgtatgaaccgttgatgattaaatgtgattattagtgagaaaataaattgtttaatcaagattttttgtacttaaataattggtacttgcagacataggggaggaaatgaaatgacaaaagagatttggaataattcgaaattgccagaattgatattatagtttatactatgctgtcactttcccaatgaaagttggtgtatcaagtttcaagaaaataattataacataatgatgatgactaaagacccatgttgctatgcttaaatggacgatcaaatttcatcgtccgtccaatttaccagtgatttcacagaacatcaatcccgtcatacttcttttctaattggttttcaaattacacatcgtttatttattcttaatcaaatagtgcatgatatgtttgacacacaaaactaaatattggacgtaagtaaatttattcggcgattcaagtaaaaagttggcatttatacttaactaatggatgtatgatatcacatgtgtttgaacatattaacttgatgtaaacaccgaatacgagtaaatacttatcaatgaaactgaccatactctttccacgagccgagcggtgtgccatttatgtccatctgccgacagccctggcacactgcggtgcaagaaacatgcaaggcccaagggcacatgaaaaaggagaggaaactcacatgctatgaaataaattcacatttgacaactccataaacagaaaatgcaaaaaaaaatgtctttatgtaccgaatttacttatttttgcacagatattatcctttcattaaaaggaacaaactaaagaacactcgatattaagaacaccaaactacaaataaacaaccataggaacatgataggtttgcgaacaataaatgcccattttcaagtacaacaaccccctccttgtgaagcagctccgtctcttccacctgaaggcccgggaatgcctccatatccaacttttattccatacgactgcaaaagaaaaaagggaagcgtaagtttcggaaaccatctattattacgtaaatgataattatacatgttggaaatcaattatcactgagatatacaagtatttactgaaatatttgcgacgtaagtttttgttttcttggttctcaaaacaataattaggacactaggattgttccaaacagaactgaaaccgataacccgaataaaaaaagttattggtttatcaatattgggttattagtttagcggttttgataacgatttagattttttttgttatcgggttatcggttcttaacggtttgaagttttttcttaacgggttaatcgataacccgatagtaaattaaataattatatttataccattttgtatataaagtctttgacttagagtttggtttcccactttcagttttggttgtctcagacacttctcgaggaaccatgcatctcaatcttaatactaaaaaatgcaacaaaattctagtggagggaccagtaccagtacattagatattatcttttattactataataatattttaattaacacgagacataggaatgtcaagtggtagcggtaggagggagttggttcagttttttagatactaggagacagaaccggaggggcccattgcaaggcccaagttgaagtccagccgtgaatcaacaaagagagggcccataatactgtcgatgagcatttccctataatacagtgtccacagttgccttccgctaagggatagccacccgctattctcttgacacgtgtcactgaaacctgctacaaataaggcaggcacctcctcattctcacactcactcactcacacagcactagttcaaacataattgtaactgatttattcttgaataacaacttcaatgaaatcaagcaacaaagctgatttcaacataaaaaaacagaacaagaaaacaaaaacagagcatcatccatcaaagtgtaatctcagcagattcaatagagactaattaacaatctgcaagactgatcgtcgcaccttcaatggagataccttcaccacctctctttggcaccagagtcaccgcaatagtctcttcatcttccaaaccaatatcctccaacagttccgttatcgccagctggaaatcaacagtcgcgatatgattagtctcaccagctctatgaacatgtggcaatctagtataactccccgcaaactccccgcggttagtctctattgaatctgctgagattacactttgatggatgatgctctgtttttgttttcttgttctgtttttttatgttgaaatcagctttgttgcttgatttcattgaagttgttattcaagaataaatcagttacaattatgtttgaggatccgttaacaagcttgttatcaactgatctctctactagagagtgtgtggtagagtgtgattgcacttgaattacagctgagatttttagttttataatactgatatcaagggtaactttatagacctgattttgtcggaatttacgagaggaccataggtatgattggtacacacgtcttgtgattgagggtattttggggatttaagcatttcttgtgattgaggctattttggggattgaaatatgacttttgtacttagcttaaaaaaaatatctaatgatgaaaaagaatatactattagaatcattatttttcgaaaaacagttactcaattttaatttgtttgaactacgttatttttcaaaaataaaattaaaagatattttgatatatttaacttatttttaatttaaaacaataaaaattcaataataaaaaaattcttatcaaattaaataaattaaactgaacaaagtattcaaaagattatttattagtgaacgtcatttcacttgtttgcacgagagttagatatgatcacatacgcttgagagaataaaaaaagagtggcggacgtgtataaaccgttgatgattaaatgtgattattagtgagaaaatgaattgtttaatcaagattttttgtatttaaataattggtatgatcacatgcgcttgagagaataaaaaaagagtgacggatgtgtatgaaccgttgatgattaaatgtgattattagtgagaaactgaattgtttaatcaagattttttgtatttaaataattggtatgatcacatgcgcttgagagaataaaaaaagagtgacagacgtgtatgaaccgttgatgattaaatgtgattattagtgagaaaataaattgtttaatcaagattttttgtacttaaataattggtacttgcagacataggggaggaaatgaaatgacaaaagagatttggaataattcgaaattgccagaattgatattatagtttatactatgctgtcactttcccaatgaaagttggtgtatcaagtttcaagaaaataattataacataatgatgatgactaaagacccatgttgctatgcttaaatggacgatcaaatttcatcgtccgtccaatttaccagtgatttcacagaacatcaatcccgtcatacttcttttctaattggttttcaaattacacatcgtttatttattcttaatcaaatagtgcatgatatgtttgacacacaaaactaaatattggacgtaagtaaatttattcggcgattcaagtaaaaagttggcatttatacttaactaatggatgtatgatatcacatgtgtttgaacatattaacttgatgtaaacaccgaatacgagtaaatacttatcaatgaaactgaccatactctttccacgagccgagcggtgtgccatttatgtccatctgccgacagccctggcacactgcggtgcaagaaacatgcaaggcccaagggcacatgaaaaaggagaggaaactcacatgctatgaaataaattcacatttgacaactccataaacagaaaatgcaaaaaaaaatgtctttatgtaccgaatttacttatttttgcacagatattatcctttcattaaaaggaacaaactaaagaacactcgatattaagaacaccaaactacaaataaacaaccataggaacatgataggtttgcgaacaataaatgcccattttcaagtacaacaaccccctccttgtgaagcagctccgtctcttccacctgaaggcccgggaatgcctccatatccaacttttattccatacgactgcaaaagaaaaaagggaagcgtaagtttcggaaaccatctattattacgtaaatgataattatacatgttggaaatcaattatcactgagatatacaagtatttactgaaatatttgcgacgtaagtttttgttttcttggttctcaaaacaataattaggacactaggattgttccaaacagaactgaaaccgataacccgaataaaaaaagttattggtttatcaatattgggttattagtttagcggttttgataacgatttagattttttttgttatcgggttatcggttcttaacggtttgaagttttttcttaacgggttaatcgataacccgatagtaaattaaataattatatttataccattttgtatataaagtctttgacttagagtttggtttcccactttcagttttggttgtctcagacacttctcgagatggtgaccgagctcaaacctgcaggaaaggtaccacattgattgagttttatatgcaatatagtaataataataatatttcttataaagcaagaggtcaatttttttttattataccaacgtcactaaattatatttgataatgtaaaacaattcaattttacttaaatatcatgaaataaactatttttataaccaaattactaaatttttccaataaaaaaaagtcattaagaagacataaaataaatttgagtaaaaagagtgaagtcgactgactttttttttttatcataagaaaataaattattaactttaacctaataaaacactaatataatttcatggaatctaatacttacctcttagaaataagaaaaagtgtttctaatagaccctcaatttacattaaatattttcaatcaaatttaaataacaaatatcaatatgaggtcaataacaatatcaaaataatatgaaaaaagagcaatacataatataagaaagaagatttaagtgcgattatcaaggtagtattatatcctaatttgctaatatttaaactcttatatttaaggtcatgttcatgataaacttgaaatgcgctatattagagcatatattaaaataaaaaaatacctaaaataaaattaagttatttttagtatatatttttttacatgacctacatttttctgggtttttctaaaggagcgtgtaagtgtcgacctcattctcctaattttccccaccacataaaaattaaaaaggaaaggtagcttttgcgtgttgttttggtacactacacctcattattacacgtgtcctcatataattggttaaccctatgaggcggtttcgtctagagtcggccatgccatctataaaatgaagctttctgcacctcaatttttcatcttctatctgatttctattataatttctattaattgccttcaaatttctctttcaaggttagaaatcttctctattttttggtttttgtctgtttagattctcgaattagctaatcaggtgctgttaaagccctaaaatttgagttttttttccgtcgaattgatgctaaaggcttaaaattagagttttttcgtcggtttgactctgaaggcctaaaatttggggttttccgggtgatttgatgataaagccctagaatttgagtttttttatttgtcggtttgatgaaaaaggccttaaatttaatttttttcccggttgatttgatgaaaaagccctagaatttgtgttttttcgtcggtttgattctaaaggcctaaaatttgagtttttccggttgttttgatgaaaaagccctaaaatttgagttttttccccgtgttttagattgtttggttttaattctcgaatcagctaatcagggagtgtgaaaagccctaaaatttgagtttttttcgttgttctgattgttgtttttatgaatttgcagattggatccatgaagcggtacaacttgcatacggcataggagaaactgaaatgccaactgtgatgaatgggcttatggtttggtgcattgaaaatggaacctcgccaaacatcaacggagtttgggttatgatggatggagatgaacaagtcgaatacccactgaaaccaatcgttgagaatgcaaaaccaacacttaggcaaatcatggcacatttctcagatgttgcagaagcgtatatagaaatgcgcaacaaaaaggaaccatatatgccacgatatggtttagttcgtaatctgcgcgatggaagtttggctcgctatgcttttgacttttatgaggtcacatcacgaacaccagtgagggctagggaagcgcacattcaaatgaaggccgcagcattgaaatcagcccaacctcgacttttcgggttggacggtggcatcagtacacaagaggagaacacagagaggcacaccaccgaggatgtttctccaagtatgcatactctacttggagtcaagaacatgtgtaacttttactcatctcctccaattatttctgatttcatgcatgtttccctacattctattatgaatcgtgttatggtgtataaacgttgtttcatatctcatctcatctattctgattttgattctcttgcctactgaatttgaccctactgtaatcggtgataaatgtgaatgcttcctcttcttcttcttcttctcagaaatcaatttctgttttgtttttgttcatctgtagacatgttcttgactccaagtagagtatgcatacttggagaaacatcctcggtggtgtgcctctctgtgttctcctcttgtgtactgatgccaccgtccaacccgaaaagtcgaggttgggctgatttcaatgctgcggccttcatttgaatgtgcgcttccctagccctcactggtgttcgtgatgtgacctcataaaagtcaaaagcatagcgagccaaacttccatcgcgcagattacgaactaaaccatatcgtggcatatatggttcctttttgttgcgcatttctatatacgcttctgcaacatctgagaaatgtgccatgatttgcctaagtgttggttttgcattctcaacgattggtttcagtgggtattcgacttgttcatctccatccatcataacccaaactccgttgatgtttggcgaggttccattttcaatgcaccaaaccataagcccattcatcacagttggcatttcagtttctcctatgccgtatgcaagttgtaccgcttcatactagtttttaatgtttagcaaatgtcctatcagttttctctttttgtcgaacggtaatttagagttttttttgctatatggattttcgtttttgatgtatgtgacaaccctcgggattgttgatttatttcaaaactaagagtttttgcttattgttctcgtctattttggatatcaatcttagttttatatcttttctagttctctacgtgttaaatgttcaacacactagcaatttggctgcagcgtatggattatggaactatcaagtctgtgggatcgataaatatgcttctcaggaatttgagattttacagtctttatgctcattgggttgagtataatatagtaaaaaaataggagctcaaacctgcaggaaaggtaccaaaccatggaaaggcgcgccgtttaaacaaactgataattgccaacacaattaacatctcaatcaaggtaaatgctttttgcttttaaagtttaaactatcagtgtttgacaggatatattggcgggtaaac

SEQ ID NO: 4 - DNA, 19 bp, synthetic construct
  misc_feature 1..19: note = Primer SPS-pSIM4363-F
  source 1..19: mol_type = other DNA
cgttcctctcaacttaatc

SEQ ID NO: 5 - DNA, 19 bp, synthetic construct
  misc_feature 1..19: note = Primer SPS-pSIM4363-R
  source 1..19: mol_type = other DNA
gcttaaattctagatcccc

SEQ ID NO: 6 - DNA, 20 bp, synthetic construct
  misc_feature 1..20: note = Primer SPS-APRT-F
  source 1..20: mol_type = other DNA
gaaccggagcaggtgaagaa

SEQ ID NO: 7 - DNA, 20 bp, synthetic construct
  misc_feature 1..20: note = Primer SPS-APRT-R
  source 1..20: mol_type = other DNA
gaagcaatcccagcgatacg

SEQ ID NO: 8 - DNA, 20 bp, synthetic construct
  misc_feature 1..20: note = PCR primer
  source 1..20: mol_type = other DNA
tctaccctccatttggtgct

SEQ ID NO: 9 - DNA, 20 bp, synthetic construct
  misc_feature 1..20: note = PCR primer
  source 1..20: mol_type = other DNA
acgggaaacgaatgtttcag

SEQ ID NO: 10 - DNA, 18 bp, synthetic construct
  misc_feature 1..18: note = PCR primer
  source 1..18: mol_type = other DNA
caatgaggttgcctggtt

SEQ ID NO: 11 - DNA, 18 bp, synthetic construct
  misc_feature 1..18: note = PCR primer
  source 1..18: mol_type = other DNA
aatcaacaatcccgaggg

SEQ ID NO: 12 - DNA, 21 bp, synthetic construct
  misc_feature 1..21: note = PCR primer
  source 1..21: mol_type = other DNA
ggcatactcatatcatccatg

SEQ ID NO: 13 - DNA, 18 bp, synthetic construct
  misc_feature 1..18: note = PCR primer
  source 1..18: mol_type = other DNA
caatccacgtgttgcaac

SEQ ID NO: 14 - DNA, 18 bp, synthetic construct
  misc_feature 1..18: note = PCR primer
  source 1..18: mol_type = other DNA
gttgcaacacgtggattg

SEQ ID NO: 15 - DNA, 20 bp, synthetic construct
  misc_feature 1..20: note = PCR primer
  source 1..20: mol_type = other DNA
ccctgaactccaactattct

SEQ ID NO: 16 - DNA, 21 bp, synthetic construct
  misc_feature 1..21: note = PCR primer
  source 1..21: mol_type = other DNA
tccactcctcatctattcttg

SEQ ID NO: 17 - DNA, 18 bp, synthetic construct
  misc_feature 1..18: note = PCR primer
  source 1..18: mol_type = other DNA
ctgcaacttcctccaaac

SEQ ID NO: 18 - DNA, 18 bp, synthetic construct
  misc_feature 1..18: note = PCR primer
  source 1..18: mol_type = other DNA
gtttggaggaagttgcag

SEQ ID NO: 19 - DNA, 18 bp, synthetic construct
  misc_feature 1..18: note = PCR primer
  source 1..18: mol_type = other DNA
ttcctgcctatgcaagac

SEQ ID NO: 20 - DNA, 19 bp, synthetic construct
  misc_feature 1..19: note = PCR primer
  source 1..19: mol_type = other DNA
ccagcaaagtgcaatctac

SEQ ID NO: 21 - DNA, 19 bp, synthetic construct
  misc_feature 1..19: note = PCR primer
  source 1..19: mol_type = other DNA
ccatcagatttgttgccac

SEQ ID NO: 22 - DNA, 21 bp, synthetic construct
  misc_feature 1..21: note = PCR primer
  source 1..21: mol_type = other DNA
gcgtacaagagttgatacctc

SEQ ID NO: 23 - DNA, 20 bp, synthetic construct
  misc_feature 1..20: note = PCR primer
  source 1..20: mol_type = other DNA
ggtcttgagaacttggaaag

SEQ ID NO: 24 - DNA, 19 bp, synthetic construct
  misc_feature 1..19: note = PCR primer
  source 1..19: mol_type = other DNA
gtggcaacaaatctgatgg

SEQ ID NO: 25 - DNA, 21 bp, synthetic construct
  misc_feature 1..21: note = PCR primer
  source 1..21: mol_type = other DNA
gaggtatcaactcttgtacgc

SEQ ID NO: 26 - DNA, 30 bp, synthetic construct
  misc_feature 1..30: note = PCR primer
  source 1..30: mol_type = other DNA
tctctcttcttctccttcaatcagtctttc

SEQ ID NO: 27 - DNA, 22 bp, synthetic construct
  misc_feature 1..22: note = PCR primer
  source 1..22: mol_type = other DNA
ttcacggctggacttcaacttg

SEQ ID NO: 28 - DNA, 20 bp, synthetic construct
  misc_feature 1..20: note = PCR primer
  source 1..20: mol_type = other DNA
cgaaaagtcttatcggagac

SEQ ID NO: 29 - DNA, 20 bp, synthetic construct
  misc_feature 1..20: note = PCR primer
  source 1..20: mol_type = other DNA
gcctcaatcacaagaaatgc

SEQ ID NO: 30 - DNA, 20 bp, synthetic construct
  misc_feature 1..20: note = PCR primer
  source 1..20: mol_type = other DNA
cactttgatggatgatgctc

SEQ ID NO: 31 - DNA, 20 bp, synthetic construct
  misc_feature 1..20: note = PCR primer
  source 1..20: mol_type = other DNA
gcctcaatcacaagaaatgc

SEQ ID NO: 32 - DNA, 31 bp, synthetic construct
  misc_feature 1..31: note = PCR primer
  source 1..31: mol_type = other DNA
aattatacatgttggaaatcaattatcactg

SEQ ID NO: 33 - DNA, 31 bp, synthetic construct
  misc_feature 1..31: note = PCR primer
  source 1..31: mol_type = other DNA
tgtcttcttaatgacttttttttattggaaa

SEQ ID NO: 34 - DNA, 29 bp, synthetic construct
  misc_feature 1..29: note = PCR primer
  source 1..29: mol_type = other DNA
acatgttcttgactccaagtagagtatgc

SEQ ID NO: 35 - DNA, 24 bp, synthetic construct
  misc_feature 1..24: note = PCR primer
  source 1..24: mol_type = other DNA
gaaataaatcaacaatcccgaggg

SEQ ID NO: 36 - DNA, 18 bp, synthetic construct
  misc_feature 1..18: note = PCR primer
  source 1..18: mol_type = other DNA
aagcagttggaggacgga

SEQ ID NO: 37 - DNA, 19 bp, synthetic construct
  misc_feature 1..19: note = PCR primer
  source 1..19: mol_type = other DNA
gaggaagatggaggtaggg

SEQ ID NO: 38 - DNA, 24 bp, synthetic construct
  misc_feature 1..24: note = PCR primer
  source 1..24: mol_type = other DNA
tggagaaacatggagatggtgagg

SEQ ID NO: 39 - DNA, 21 bp, synthetic construct
  misc_feature 1..21: note = PCR primer
  source 1..21: mol_type = other DNA
tgcttctcaggaatttgagat

SEQ ID NO: 40 - DNA, 20 bp, synthetic construct
  misc_feature 1..20: note = PCR primer
  source 1..20: mol_type = other DNA
gatttgatttggtcgtgaga

SEQ ID NO: 41 - DNA, 27 bp, synthetic construct
  misc_feature 1..27: note = PCR primer
  source 1..27: mol_type = other DNA
acagtctttatgctcattgggttgagt

SEQ ID NO: 42 - DNA, 19 bp, synthetic construct
  misc_feature 1..19: note = PCR primer
  source 1..19: mol_type = other DNA
cgttcctctcaacttaatc

SEQ ID NO: 43 - DNA, 19 bp, synthetic construct
  misc_feature 1..19: note = PCR primer
  source 1..19: mol_type = other DNA
gcttaaattctagatcccc

SEQ ID NO: 44 - DNA, 23 bp, synthetic construct
  misc_feature 1..23: note = PCR primer
  source 1..23: mol_type = other DNA
tgccaaaaacgagcaacaacaac

SEQ ID NO: 45 - DNA, 18 bp, synthetic construct
  misc_feature 1..18: note = PCR primer
  source 1..18: mol_type = other DNA
atggtagaactggaggag

SEQ ID NO: 46 - DNA, 20 bp, synthetic construct
  misc_feature 1..20: note = PCR primer
  source 1..20: mol_type = other DNA
ttccatggtttggtaccttt

SEQ ID NO: 47 - DNA, 20 bp, synthetic construct
  misc_feature 1..20: note = PCR primer
  source 1..20: mol_type = other DNA
cgaggggaggttcggtgatt

SEQ ID NO: 48 - DNA, 23 bp, synthetic construct
  misc_feature 1..23: note = PCR primer
  source 1..23: mol_type = other DNA
tctcagctgtaattcaagtgcaa

SEQ ID NO: 49 - DNA, 18 bp, synthetic construct
  misc_feature 1..18: note = PCR primer
  source 1..18: mol_type = other DNA
gccggaatcgggttgatc

SEQ ID NO: 50 - DNA, 22 bp, synthetic construct
  misc_feature 1..22: note = PCR primer
  source 1..22: mol_type = other DNA
cacactctaccacacactctct

SEQ ID NO: 51 - DNA, 21 bp, synthetic construct
  misc_feature 1..21: note = PCR primer
  source 1..21: mol_type = other DNA
ttgaggatccgttaacaagct

SEQ ID NO: 52 - DNA, 23 bp, synthetic construct
  misc_feature 1..23: note = PCR primer
  source 1..23: mol_type = other DNA
tctcagctgtaattcaagtgcaa

SEQ ID NO: 53 - DNA, 22 bp, synthetic construct
  misc_feature 1..22: note = PCR primer
  source 1..22: mol_type = other DNA
cacactctaccacacactctct

SEQ ID NO: 54 - DNA, 18 bp, synthetic construct
  misc_feature 1..18: note = PCR primer
  source 1..18: mol_type = other DNA
caaaccataagcccattc

SEQ ID NO: 55 - DNA, 20 bp, synthetic construct
  misc_feature 1..20: note = PCR primer
  source 1..20: mol_type = other DNA
ctctaaattaccgttcgaca

SEQ ID NO: 56 - DNA, 22 bp, synthetic construct
  misc_feature 1..22: note = PCR primer
  source 1..22: mol_type = other DNA
atttcagtttctcctatgccgt

SEQ ID NO: 57 - DNA, 20 bp, synthetic construct
  misc_feature 1..20: note = PCR primer
  source 1..20: mol_type = other DNA
ccttgggcgaaatataaaga

SEQ ID NO: 58 - DNA, 19 bp, synthetic construct
  misc_feature 1..19: note = PCR primer
  source 1..19: mol_type = other DNA
gaagctatgaaagggaatg

SEQ ID NO: 59 - DNA, 18 bp, synthetic construct
  misc_feature 1..18: note = PCR primer
  source 1..18: mol_type = other DNA
agccggagatggatgtgt

SEQ ID NO: 60 - DNA, 20 bp, synthetic construct
  misc_feature 1..20: note = PCR primer
  source 1..20: mol_type = other DNA
tagatgtcaaggtgaggtgg

SEQ ID NO: 61 - DNA, 21 bp, synthetic construct
  misc_feature 1..21: note = PCR primer
  source 1..21: mol_type = other DNA
ggagggtagcatgttgattgt

SEQ ID NO: 62 - DNA, 18 bp, synthetic construct
  misc_feature 1..18: note = PCR primer
  source 1..18: mol_type = other DNA
agcgttgctgcctgtgat

SEQ ID NO: 63 - DNA, 19 bp, synthetic construct
  misc_feature 1..19: note = PCR primer
  source 1..19: mol_type = other DNA
tgtacagtctatgcctcgg

SEQ ID NO: 64 - DNA, 18 bp, synthetic construct
  misc_feature 1..18: note = PCR primer
  source 1..18: mol_type = other DNA
tgctgcgtaacatcgttg

SEQ ID NO: 65 - DNA, 18 bp, synthetic construct
  misc_feature 1..18: note = PCR primer
  source 1..18: mol_type = other DNA
acgcgcttgctgcttgga

SEQ ID NO: 66 - DNA, 18 bp, synthetic construct
  misc_feature 1..18: note = PCR primer
  source 1..18: mol_type = other DNA
aagcagttggaggacgga

SEQ ID NO: 67 - DNA, 19 bp, synthetic construct
  misc_feature 1..19: note = PCR primer
  source 1..19: mol_type = other DNA
gaggaagatggaggtaggg

SEQ ID NO: 68 - DNA, 24 bp, synthetic construct
  misc_feature 1..24: note = PCR primer
  source 1..24: mol_type = other DNA
tggagaaacatggagatggtgagg

SEQ ID NO: 69 - DNA, 19 bp, synthetic construct
  misc_feature 1..19: note = PCR primer
  source 1..19: mol_type = other DNA
ttatcgataccgtcgacct

SEQ ID NO: 70 - DNA, 20 bp, synthetic construct
  misc_feature 1..20: note = PCR primer
  source 1..20: mol_type = other DNA
gatttgatttggtcgtgaga

SEQ ID NO: 71 - DNA, 24 bp, synthetic construct
  misc_feature 1..24: note = PCR primer
  source 1..24: mol_type = other DNA
acaccctacattctactcgagtca

SEQ ID NO: 72 - DNA, 18 bp, synthetic construct
  misc_feature 1..18: note = PCR primer
  source 1..18: mol_type = other DNA
aaccggtaaacctaggaa

SEQ ID NO: 73 - DNA, 19 bp, synthetic construct
  misc_feature 1..19: note = PCR primer
  source 1..19: mol_type = other DNA
aaagagagtagcttccaca

SEQ ID NO: 74 - DNA, 22 bp, synthetic construct
  misc_feature 1..22: note = PCR primer
  source 1..22: mol_type = other DNA
agcaaacaattgaaagggcccg

SEQ ID NO: 75 - DNA, 20 bp, synthetic construct
  misc_feature 1..20: note = PCR primer
  source 1..20: mol_type = other DNA
gttctttggtgctatcttgt

SEQ ID NO: 76 - DNA, 19 bp, synthetic construct
  misc_feature 1..19: note = PCR primer
  source 1..19: mol_type = other DNA
gcttaaattctagatcccc

SEQ ID NO: 77 - DNA, 23 bp, synthetic construct
  misc_feature 1..23: note = PCR primer
  source 1..23: mol_type = other DNA
tgccaaaaacgagcaacaacaac

SEQ ID NO: 78 - DNA, 18 bp, synthetic construct
  misc_feature 1..18: note = PCR primer
  source 1..18: mol_type = other DNA
atggtagaactggaggag

SEQ ID NO: 79 - DNA, 18 bp, synthetic construct
  misc_feature 1..18: note = PCR primer
  source 1..18: mol_type = other DNA
aggtcgactctagaggat

SEQ ID NO: 80 - DNA, 20 bp, synthetic construct
  misc_feature 1..20: note = PCR primer
  source 1..20: mol_type = other DNA
cgaggggaggttcggtgatt

SEQ ID NO: 81 - DNA, 20 bp, synthetic construct
  misc_feature 1..20: note = PCR primer
  source 1..20: mol_type = other DNA
cgcaaagagggaactaaaca

SEQ ID NO: 82 - DNA, 19 bp, synthetic construct
  misc_feature 1..19: note = PCR primer
  source 1..19: mol_type = other DNA
agattgagatgcatggttc

SEQ ID NO: 83 - DNA, 23 bp, synthetic construct
  misc_feature 1..23: note = PCR primer
  source 1..23: mol_type = other DNA
agcgaaaaatagggataacaggg

SEQ ID NO: 84 - DNA, 23 bp, synthetic construct
  misc_feature 1..23: note = PCR primer
  source 1..23: mol_type = other DNA
tctcagctgtaattcaagtgcaa

SEQ ID NO: 85 - DNA, 18 bp, synthetic construct
  misc_feature 1..18: note = PCR primer
  source 1..18: mol_type = other DNA
gccggaatcgggttgatc

SEQ ID NO: 86 - DNA, 22 bp, synthetic construct
  misc_feature 1..22: note = PCR primer
  source 1..22: mol_type = other DNA
cacactctaccacacactctct

SEQ ID NO: 87 - DNA, 21 bp, synthetic construct
  misc_feature 1..21: note = PCR primer
  source 1..21: mol_type = other DNA
acaattatgtttgaggatccg

SEQ ID NO: 88 - DNA, 23 bp, synthetic construct
  misc_feature 1..23: note = PCR primer
  source 1..23: mol_type = other DNA
tctcagctgtaattcaagtgcaa

SEQ ID NO: 89 - DNA, 22 bp, synthetic construct
  misc_feature 1..22: note = PCR primer
  source 1..22: mol_type = other DNA
cacactctaccacacactctct

SEQ ID NO: 90 - DNA, 19 bp, synthetic construct
  misc_feature 1..19: note = PCR primer
  source 1..19: mol_type = other DNA
cagctttgttgcttgattt

SEQ ID NO: 91 - DNA, 18 bp, synthetic construct
  misc_feature 1..18: note = PCR primer
  source 1..18: mol_type = other DNA
acctcctcattctcacac

SEQ ID NO: 92 - DNA, 26 bp, synthetic construct
  misc_feature 1..26: note = PCR primer
  source 1..26: mol_type = other DNA
cactcacacagcactagttcaaacat

SEQ ID NO: 93 - DNA, 18 bp, synthetic construct
  misc_feature 1..18: note = PCR primer
  source 1..18: mol_type = other DNA
caaaccataagcccattc

SEQ ID NO: 94 - DNA, 18 bp, synthetic construct
  misc_feature 1..18: note = PCR primer
  source 1..18: mol_type = other DNA
atcagggagtgtgaaaag

SEQ ID NO: 95 - DNA, 20 bp, synthetic construct
  misc_feature 1..20: note = PCR primer
  source 1..20: mol_type = other DNA
agattggatccatgaagcgg

SEQ ID NO: 96 - DNA, 18 bp, synthetic construct
  misc_feature 1..18: note = PCR primer
  source 1..18: mol_type = other DNA
caaaccataagcccattc

SEQ ID NO: 97 - DNA, 20 bp, synthetic construct
  misc_feature 1..20: note = PCR primer
  source 1..20: mol_type = other DNA
ctctaaattaccgttcgaca

SEQ ID NO: 98 - DNA, 22 bp, synthetic construct
  misc_feature 1..22: note = PCR primer
  source 1..22: mol_type = other DNA
atttcagtttctcctatgccgt

SEQ ID NO: 99 - DNA, 22 bp, synthetic construct
  misc_feature 1..22: note = PCR primer
  source 1..22: mol_type = other DNA
ctgggtcaagtacaaaggcaac

SEQ ID NO: 100 - DNA, 20 bp, synthetic construct
  misc_feature 1..20: note = PCR primer
  source 1..20: mol_type = other DNA
cattttggggtccggtccaa

SEQ ID NO: 101 - DNA, 22 bp, synthetic construct
  misc_feature 1..22: note = PCR primer
  source 1..22: mol_type = other DNA
cggttctggttcctccacccgg

(19) United States
(12) Patent Application Publication
(10) Pub. No.: US 2023/0070527 A1
(43) Pub. Date: Mar. 9, 2023

(54) POTATO TRANSFORMATION VECTORS

(71) Applicant: J.R. Simplot Company, Boise, ID (US)

(72) Inventors: Nicolas CHAMPOURET, Boise, ID (US); Craig RICHAEL, Boise, ID (US); Hui DUAN, Boise, ID (US)

(21) Appl. No.: 17/822,696

(22) Filed: Aug. 26, 2022

Related U.S. Application Data
(63) Continuation of application No. PCT/US2021/020075, filed on Feb. 26, 2021.
(60) Provisional application No. 62/983,502, filed on Feb. 28, 2020.

(51) Int. Cl. C12N 15/82; A01H 6/82

(52) U.S. Cl. CPC C12N 15/8282; C12N 15/8243; C12N 15/8283; A01H 6/827; C12N 2330/51

(57) ABSTRACT

The present disclosure provides plant transformation vectors, T-DNA insert regions, and transformed plants. The vectors are designed to be a binary vector for use in plant transformations for such as potato. The transformed plants are characterized in that they contain the T-DNA insert region comprising stacked expression cassettes and the corresponding phenotype. The present disclosure also provides methods for identifying genetic material in transformed plants, including in food products made from such plants. The disclosure further relates to the materials and/or means for detecting plant transformation events and methods for detecting presence of plant transformation events.

### CROSS-REFERENCE TO RELATED APPLICATIONS

This application is a continuation of PCT/US2021/020075, filed Feb. 26, 2021, which claims the benefit of priority to U.S. Provisional Application No. 62/983,502 filed on Feb. 28, 2020, which is hereby incorporated by reference in its entirety.

### FIELD

The present disclosure relates to a genetic construct and/or vector for plant transformation, plants transformed with the construct and/or vector. Also, provided are methods for transforming plants with the constructs and/or vector and detecting plant transformation events.

### REFERENCE TO AN ELECTRONIC SEQUENCE LISTING

The contents of the electronic sequence listing (JRSI_083_01US_SeqList_ST26.xml; Size: 230,013 bytes; and Date of Creation: Aug. 25, 2022) is herein incorporated by reference in its entirety.

### BACKGROUND

Over the past 20 years rapid scientific advances in molecular and cell biology have resulted in the development of technology to enable genetic engineering of plants (development of transformed plants, transgenic plants or GMOs). This offers new opportunities for the incorporation of genes into plants and represents a new technology platform for the next level of genetic gain in plant breeding.

However, the assembly of large transformation constructs that carry multiple genes and efficiently transform plants has been a challenge because of instability problems in bacteria and/or technical difficulty of efficiently generating stable transgenic plants with the expected functional phenotypes, as the insertion of the transgene is a random event.

Thus, there is a need to develop plant transformation vectors with multiple transgenes within a T-DNA insert region. Also, a further need in the art is to produce plant varieties with desired agronomic traits, especially in potato, such as increased black spot bruise tolerance, lowered reducing sugars, increased resistance to pests and pathogens, insensitivity to herbicides, low acrylamide content, and the like.

### SUMMARY OF THE DISCLOSURE

The following embodiments and aspects thereof are described in conjunction with systems, tools and methods which are meant to be exemplary, not limiting in scope. In various embodiments, one or more of the above-described problems have been reduced or eliminated, while other embodiments are directed to other improvements.

The present disclosure provides plant transformation vectors with T-DNA insert regions to generate new plants with commercially desired traits. In some embodiments, the vectors are designed to provide new traits, for example, increased black spot bruise tolerance, lowered reducing sugars, increased resistance to pests and pathogens, insensitivity to herbicides, low acrylamide content, and the like, in plant transformants. In some embodiments, the transformed plants contains the T-DNA insert regions comprising stacked expression cassettes and the corresponding phenotype.

The present disclosure also provides methods for using the T-DNA insert regions for producing new plant events with newly-acquired traits taught herein and identifying genetic material in transformed plants, including in food products made from such plants. The disclosure further provides the materials and/or means for detecting plant transformation events and methods for detecting presence of plant transformation events.

In some embodiments, the present disclosure provides a transfer-DNA (T-DNA) characterized by a plurality of expression cassettes, comprising: at least one resistance (R) gene to potato late blight; a polynucleotide for decreasing acrylamide; a polynucleotide for reducing black spot; and a polynucleotide conferring protection to potato virus Y, wherein each expression cassette comprises at least one promoter. Also the present disclosure teaches that the T-DNA further comprises an expression cassette comprising a selection marker, a polynucleotide conferring protection to Potato virus X, a polynucleotide conferring protection to Potato leaf roll virus, a polynucleotide conferring protection to tobacco rattle virus, a polynucleotide conferring protection to verticillium wilt, and/or a polynucleotide conferring protection to a parasitic nematode.

Provided herein are the resistance (R) genes selected from the group consisting of Rpi-Vnt1, Rpi-Amr3, Rpi-B1b2, RpiBlb1, Rpi-mcq1, Rpi-Amr1, and Rpi-chc1.

Provided herein is the polynucleotide for decreasing acrylamide that is a silencing element selected from the group consisting of: an inverted repeat of a vacuolar invertase (VInv) gene fragment, an inverted repeat of asparagine synthetase-1 gene (Asn1) fragment, an inverted repeat of asparagine synthetase-2 gene (Asn2) fragment, an inverted repeat of phosphorylase-L (PhL) fragment, and an inverted repeat of water dikinase-(R1) gene fragment.

Provided herein is the polynucleotide for reducing black spot that is a silencing element comprising an inverted repeat of a polyphenol oxidase gene (Ppo) fragment.

Also provided herein is the polynucleotide for decreasing acrylamide that lowers the level of reducing sugars, which confers resistance to cold-induced sweetening.

In some embodiments, the disclosure provides a potato transformation vector designated pSIM4363, wherein the vector includes a T-DNA region comprising the nucleotide sequence set forth in SEQ ID NO:2, and wherein the plant transformation vector was deposited under ATCC Accession No. PTA-126737.

In other embodiments, the disclosure provides a potato transformation vector designated pSIM4617, wherein the vector includes a T-DNA region comprising the nucleotide sequence set forth in SEQ ID NO:3, and wherein the plant transformation vector was deposited under ATCC Accession No. PTA-126738.

In some embodiments, the disclosure provides a potato plant, a part thereof, or a cell thereof, comprising the T-DNA described herein, wherein the plant exhibits: resistance to potato late blight caused by Phytophthora infestans; increased black spot bruise tolerance; decreased acrylamide; and resistance to potato virus Y.

In some embodiments, the disclosure provides a commodity plant product produced from the potato plant described herein, wherein the product comprises the T-DNA.

Also provided is a potato plant, tuber, or a part thereof, comprising the T-DNA insert region of the plant transformation vector pSIM4363, wherein vector pSMIM4363 was deposited under ATCC Accession No. PTA-126737.

Also provided is potato plant, tuber, or a part of a tuber, comprising the T-DNA insert region of the plant transformation vector pSIM4617, wherein vector pSIM4617 was deposited under ATCC Accession No. PTA-126738.

In some embodiments, the present disclosure teaches a food product made from the potato tuber taught herein or a heat-processed tuber product obtained from the potato tuber taught herein.

In some embodiments, the present disclosure teaches a method for detecting presence of a target insert region in a nucleic acid sample, the method comprising: (i) isolating the nucleic acid sample from a plant, or plant part, or plant-derived food product; (ii) screening presence of the target insert region of pSIM4363 or pSIM4617, and (iii) selecting a transformed plant with the target insert region.

In addition to the exemplary aspects and embodiments described above, further aspects and embodiments will become apparent by the following descriptions.

### BRIEF DESCRIPTION OF THE DRAWINGS

FIG. 1 depicts the pSIM4363 transformation vector (SEQ ID NO: 1). The vector backbone region, on the left, is 8,925 bp long, as it starts at position 29,957 bp and ends at position 38,881 bp. The backbone DNA consists mainly of bacterial DNA which provides support maintenance of the T-DNA insert prior to plant transformation. The T-DNA insert region, including flanking Left and Right border sequences, is 29,956 bp long (from 1 bp to 29,956 bp).

FIG. 2 provides a schematic representation of the T-DNA insert region (SEQ ID NO:2) of the pSIM4363 transformation vector, which comprises six expression cassettes designed to express gene coding sequences and/or inverted repeats (silencing elements). Also, FIG. 2 provides positions of primers designed to amplify a region that is specific to a unique junction between cassettes within the pSIM4363 T-DNA, namely the Amr3 terminator and the Blb2 terminator sequences.

FIG. 3 provides a schematic representation of the T-DNA insert region (SEQ ID NO:3) of the pSIM4617 transformation vector, which comprises seven expression cassettes designed to express gene coding sequences and/or inverted repeats (silencing elements). Also, primers presented in FIG. 2 can be used to detect the pSIM4617 construct and transformed events because the junction between Rpi-amr3 and Rpi-blb2 is conserved between the two constructs; pSIM4363 and pSIM4617.

FIG. 4 displays results of PCR to test pSIM4363-primer specificity using 22.5 ng of DNA isolated from pSIM4363-transformed events (BG-115, NA-16), pSIM1278-transformed events (E12, Y9), pSIM1678-transformed event (Y9), and conventional controls Russet Burbank (RB), and Russet Norkotah TX 296 (NK). pSIM4363 primers were shown to only amplify DNA samples containing pSIM4363. The same samples were tested against APRT primers to identify endogenous APRT gene amplification. Arrows are shown to indicate (i) PCR products at 220 bp for pSIM4363 positive samples and (ii) PCR products at 121 bp for endogenous APRT positive samples. pSIM4363 (p4363), pSIM1278 (p1278), and pSIM1678 (p1678) plasmid DNA were used as positive controls. NTC (no template controls) were also included.

FIG. 5 depicts glucose concentration in Russet Norkotah lines transformed with the pSIM4363 construct (NA lines) compared to various controls stored at two cold storage temperatures and from two growing sites—one in Idaho and one in Washington. NK 296=Russet Norkotah untransformed control. NK p4382-3=an VInv-silenced control line known to be low in VInv activity. E12=a Russet Burbank empty vector control. X17=a Russet Ranger transgenic known to have low VInv activity.

### DETAILED DESCRIPTION

### Definitions

In the description and tables which follow, a number of terms are used. In order to provide a clear and consistent understanding of the specification and claims, including the scope to be given such terms, the following definitions are provided.

The term “a” or “an” refers to one or more of that entity; for example, “a primer” refers to one or more primers or at least one primer. As such, the terms “a” (or “an”), “one or more” and “at least one” are used interchangeably herein. In addition, reference to “an element” by the indefinite article “a” or “an” does not exclude the possibility that more than one of the elements is present, unless the context clearly requires that there is one and only one of the elements.

Artificially manipulated. as used herein, “artificially manipulated” means to move, arrange, operate or control by the hands or by mechanical means or recombinant means, such as by genetic engineering techniques, a plant or plant cell, so as to produce a plant or plant cell that has a different biological, biochemical, morphological, or physiological phenotype and/or genotype in comparison to unmanipulated, naturally-occurring counterpart.

Backbone. Nucleic acid sequence of a vector that excludes the DNA insert sequence intended for transfer.

Bacterial Ring Rot. Bacterial ring rot is a disease caused by the bacterium Clavibacter michiganense ssp. Bacterial ring rot derives its name from a characteristic breakdown of the vascular ring within the tuber. This ring often appears as a creamy-yellow to light-brown, cheesy rot. On the outer surface of the potato, severely diseased tubers may show slightly sunken, dry and cracked areas. Symptoms of bacterial ring rot in the vascular tissue of infected tubers can be less obvious than described above, appearing as only a broken, sporadically appearing dark line or as a continuous, yellowish discoloration.

Black spot bruise. Black spots found in bruised tuber tissue are a result of a pigment called melanin that is produced following the injury of cells and gives tissue a brown, gray or black appearance. Melanin is formed when phenol substrates and an appropriate enzyme come in contact with each other as a result of cellular damage. The damage does not require broken cells. However, mixing of the substrate and enzyme must occur, usually when the tissue is impacted. Black spots occur primarily in the perimedullary tissue just beneath the vascular ring, but may be large enough to include a portion of the cortical tissue.

Border-like sequences. A “border-like” sequence is isolated from the selected plant species that is to be modified and functions like the border sequences of Agrobacterium. That is, a border-like sequence of the present disclosure promotes and facilitates the integration of a polynucleotide to which it is linked. A border-like sequence of a DNA insert is between 5-100 bp in length, 10-80 bp in length, 15-75 bp in length, 15-60 bp in length, 15-50 bp in length, 15-40 bp in length, 15-30 bp in length, 16-30 bp in length, 20-30 bp in length, 21-30 bp in length, 22-30 bp in length, 23-30 bp in length, 24-30 bp in length, 25-30 bp in length, or 26-30 bp in length. A DNA insert left and right border sequence are isolated from and/or native to the genome of a plant that is to be modified. A DNA insert border-like sequence is not identical in nucleotide sequence to any known Agrobacterium-derived T-DNA border sequence. Thus, a DNA insert border-like sequence may possess 1, 2, 3, 4, 5, 6, 7, 8, 9, 10, 11, 12, 13, 14, 15, 16, 17, 18, 19, 20, or more nucleotides that are different from a T-DNA border sequence from an Agrobacterium species, such as Agrobacterium tumefaciens or Agrobacterium rhizogenes. That is, a DNA insert border, or a border-like sequence of the present disclosure has at least 95%, at least 90%, at least 80%, at least 75%, at least 70%, at least 60% or at least 50% sequence identity with a T-DNA border sequence from an Agrobacterium species, such as Agrobacterium tumefaciens or Agrobacterium rhizogenes, but not 100% sequence identity. As used herein, the descriptive terms “DNA insert border” and “DNA insert border-like” are exchangeable. A border-like sequence can be isolated from a plant genome and be modified or mutated to change the efficiency by which it is capable of integrating a nucleotide sequence into another nucleotide sequence. Other polynucleotide sequences may be added to or incorporated within a border-like sequence of the present disclosure. Thus, a DNA insert left border or a DNA insert right border may be modified so as to possess 5′- and 3′-multiple cloning sites, or additional restriction sites. A DNA insert border sequence may be modified to increase the likelihood that backbone DNA from the accompanying vector is not integrated into the plant genome.

Cotyledon. A cotyledon is a type of seed leaf. The cotyledon contains the food storage tissues of the seed.

Degenerate primer. A “degenerate primer” is an oligonucleotide that contains sufficient nucleotide variations that it can accommodate base mismatches when hybridized to sequences of similar, but not exact, homology.

Embryo. The embryo is the immature plant contained within a mature seed.

Event. Event refers to the unique DNA recombination event that took place in one plant cell, which was then used to generate entire transgenic plants. Plant cells are transformed with a vector carrying a DNA insert of interest (for example, T-DNA). Transformed cells are regenerated into transgenic plants, and each resulting transgenic plant represents a unique event. Molecular techniques such as Southern blot hybridization or PCR are used to confirm each transformed event. Different events possess differences in the number of copies of DNA insert in the cell genome, the arrangement of the DNA insert copies and/or the DNA insert location in the genome.

Expression cassette. “Expression cassette” refers to a DNA sequence capable of directing transcription of a particular nucleotide sequence in an appropriate host cell, comprising a promoter operably linked to the nucleotide sequence of interest which is optionally operably linked to termination signals. The particular nucleotide sequence can code for a protein of interest but can also code for a functional RNA of interest, for example antisense RNA or a non-translated RNA, in the sense or antisense direction for silencing, repressing, downregulating, or knocking down. The expression cassette comprising the nucleotide sequence of interest may be chimeric, meaning that at least one of its components is heterologous with respect to at least one of its other components. The expression cassette may also be one which is naturally occurring but has been obtained in a recombinant form useful for heterologous expression. The expression of the nucleotide sequence in the expression cassette may be under the control of a constitutive promoter or of an inducible promoter which initiates transcription only when the host cell is exposed to some particular external stimulus. In the case of a multicellular organism, the promoter can also be specific to a particular tissue or organ or stage of development.

Foreign. “Foreign” with respect to a nucleic acid is non-native nucleic acid.

Single locus Converted (Conversion). Single locus converted (conversion) plant refers to plants wherein essentially all of the desired morphological and physiological characteristics of a variety are recovered in addition to the one or more loci transferred into the variety via, for example, the backcrossing technique, via genetic engineering or via mutation.

Genetic rearrangement. Refers to the re-association of genetic elements that can occur spontaneously in vivo as well as in vitro which introduce a new organization of genetic material. For instance, the splicing together of polynucleotides at different chromosomal loci, can occur spontaneously in vivo during both plant development and sexual recombination. Accordingly, recombination of genetic elements by non-natural genetic modification techniques in vitro is akin to recombination events that also can occur through sexual recombination in vivo.

Golden nematode. Globodera rostochiensis, commonly known as golden nematode, is a plant parasitic nematode affecting the roots and tubers of potato plants. Symptoms include poor plant growth, wilting, water stress and nutrient deficiencies.

Hypocotyl. A hypocotyl is the portion of an embryo or seedling between the cotyledons and the root. Therefore, it can be considered a transition zone between shoot and root.

Isolated. “Isolated” refers to any nucleic acid or compound that is physically separated from its normal, native environment. The isolated material may be maintained in a suitable solution containing, for instance, a solvent, a buffer, an ion, or other component, and may be in purified, or unpurified, form. Isolated may also refer to nucleic acid that has been isolated from an organism and is maintained, for example, in a plasmid.

Late blight. A potato disease caused by the oomycete Phytophthora infestans and also known as ‘potato blight’ that can infect and destroy the leaves, stems, fruits, and tubers of potato plants.

Marketable Yield. Marketable yield is the weight of all tubers harvested that are between 2 and 4 inches in diameter. Marketable yield is measured in cwt (hundred weight) where cwt=100 pounds.

Native. A “native” genetic element refers to a nucleic acid that naturally exists in, originates from, or belongs to the genome of a plant that is to be transformed.

Non-natural nucleotide junction. “Non-natural nucleotide junction” or “non-naturally occurring nucleotide junction” refers to a sequence of nucleotides that do not occur in nature. Rather, these sequences are formed via a genetic transformation event. For example, the genetic transformation events described herein may be created with expression cassettes that contain no non-native potato DNA. Thus, these non-natural nucleotide junctions are composed of potato nucleotides, but these nucleotides are in a genetic arrangement that does not occur in nature, but which results from the manipulation of man that occurs during the genetic transformation of the potato.

Operably linked. Combining two or more molecules in such a fashion that in combination they function properly in a plant cell. For instance, a promoter is operably linked to a structural gene when the promoter controls transcription of the structural gene.

Plant. As used herein, the term “plant” includes a monocot or a dicot. The word “plant,” as used herein, also encompasses plant cells, seed, plant progeny, propagule whether generated sexually or asexually, and descendants of any of these, such as cuttings or seed. Plant cells include suspension cultures, callus, embryos, meristematic regions, callus tissue, leaves, roots, shoots, gametophytes, sporophytes, pollen, seeds and microspores.

Plant Parts. As used herein, the term “plant parts” (or a potato plant, or a part thereof) includes but is not limited to protoplast, leaf, stem, root, root tip, anther, pistil, seed, embryo, pollen, ovule, cotyledon, hypocotyl, flower, tuber, eye, tissue, petiole, cell, meristematic cell, and the like.

Progeny. As used herein, includes an F₁ potato plant produced from the cross of two potato plants where at least one plant includes the selected event and progeny further includes, but is not limited to, subsequent F₂, F₃, F₄, F₅, F₆, F₇, F₈, F₉, generations.

Quantitative Trait Loci (QTL). Quantitative trait loci (QTL) refer to genetic loci that control to some degree numerically representable traits that are usually continuously distributed.

Recombinant. As used herein, broadly describes various technologies whereby genes can be cloned, DNA can be sequenced, and protein products can be produced. As used herein, the term also describes proteins that have been produced following the transfer of genes into the cells of plant host systems.

Regeneration. Regeneration refers to the development of a plant from tissue culture.

Regulatory sequences. Refers to those sequences which are standard and known to those in the art that may be included in the expression vectors to increase and/or maximize transcription of a gene of interest or translation of the resulting RNA in a plant system. These include, but are not limited to, promoters, peptide export signal sequences, introns, polyadenylation, and transcription termination sites.

RNA interference. “RNA interference” (“RNAi”) refers to a selective intracellular degradation of RNA. RNAi occurs in cells naturally to remove foreign RNAs. Natural RNAi proceeds via fragments cleaved from free dsRNA which direct the degradative mechanism to other similar RNA sequences. In the present disclosure, RNAi can be initiated by the hand of man, for example, to silence the expression of target genes. A RNAi agent having a strand which has a “sequence sufficiently complementary to a target mRNA sequence to direct target-specific RNA interference (RNAi)” means that the strand has a sequence sufficient to trigger the destruction of the target mRNA by the RNAi machinery or process.

Selectable marker. A “selectable marker” is used to identify transformation events. Examples of selectable markers include, but are not limited to, the streptomycin phosphotransferase (spt) gene encoding streptomycin resistance, the phosphomannose isomerase (pmi) gene that converts mannose-6-phosphate into fructose-6 phosphate; the neomycin phosphotransferase (nptII) gene encoding kanamycin and geneticin resistance, the kanamycin resistance gene (KmR); the hygromycin phosphotransferase (hpt or aphiv) gene encoding resistance to hygromycin, acetolactate synthase (als) genes encoding resistance to sulfonylurea-type herbicides, genes coding for resistance to herbicides which act to inhibit the action of glutamine synthase such as phosphinothricin or basta (e.g., the bar gene), or other similar genes known in the art.

Sense suppression. Reduction in expression of an endogenous gene by expression of one or more additional copies of all or part of that gene in transgenic plants.

Specific gravity. As used herein, “specific gravity” is an expression of density and is a measurement of potato quality. There is a high correlation between the specific gravity of the tuber and the starch content and percentage of dry matter or total solids. A higher specific gravity contributes to higher recovery rate and better quality of the processed product.

Sequence identity. “sequence identity” or “identity” in the context of two nucleic acid or polypeptide sequences includes reference to the number of residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988). The comparison of sequences and determination of percent identity between two sequences can be accomplished using a mathematical algorithm. An example of a local alignment algorithm utilized for the comparison of sequences is the NCBI Basic Local Alignment Search Tool (BLAST®) (Altschul et al. 1990 J. Mol. Biol. 215: 403-10), which is available from several sources, including the National Center for Biotechnology Information (NCBI, Bethesda, Md.) and on the Internet, for use in connection with the sequence analysis programs blastp, blastn, blastx, tblastn and tblastx. It can be accessed on the internet via the National Library of Medicine (NLM)'s world-wide-web URL. A description of how to determine sequence identity using this program is available at the NLM's website on BLAST tutorial. Another example of a mathematical algorithm utilized for the global comparison of sequences is the Clustal W and Clustal X (Larkin et al. 2007 Bioinformatics, 23, 2947-294, Clustal W and Clustal X version 2.0) as well as Clustal omega. Unless otherwise stated, references to sequence identity used herein refer to the NCBI Basic Local Alignment Search Tool (BLAST®).

Silencing element. “Silencing element” in the present disclosure refers to a polynucleotide that is capable of reducing or eliminating the level of expression of a target polynucleotide or the polypeptide encoded thereby.

T-DNA. According to the present disclosure, T-DNA refers to transfer DNA, usually maintained in a vector and used, for example, to transform plants.

T-DNA-Like. A “T-DNA-like” sequence is a nucleic acid that is isolated from a selected plant species, and which shares at least 75%, 80%, 85%, 90%, or 95%, but not 100%, sequence identity with Agrobacterium species T-DNA. The T-DNA-like sequence may contain one or more border or border-like sequences that are each capable of integrating a nucleotide sequence into another polynucleotide.

Total Yield. Total yield refers to the total weight of all harvested tubers.

Transformation of plant cells. A process by which DNA is integrated into the genome of a plant cell. The integration may be transient or stable. “Stably” refers to the permanent, or non-transient retention and/or expression of a polynucleotide in and by a cell genome. Thus, a stably integrated polynucleotide is one that is a fixture within a transformed cell genome and can be replicated and propagated through successive progeny of the cell or resultant transformed plant. Transformation may occur under natural or artificial conditions using various methods well known in the art. Transformation may rely on any known method for the insertion of nucleic acid sequences into a prokaryotic or eukaryotic host cell, including Agrobacterium-mediated transformation protocols, viral infection, whiskers, electroporation, heat shock, lipofection, polyethylene glycol treatment, micro-injection, and particle bombardment.

Transgenic plant. A genetically modified plant which contains at least one transgene.

Variant. A “variant,” as used herein, is understood to mean a nucleotide or amino acid sequence that deviates from the standard, or given, nucleotide or amino acid sequence of a particular gene or protein. The terms, “isoform,” “isotype,” and “analog” also refer to “variant” forms of a nucleotide or an amino acid sequence. An amino acid sequence that is altered by the addition, removal or substitution of one or more amino acids, or a change in nucleotide sequence, may be considered a “variant” sequence. The variant may have “conservative” changes, wherein a substituted amino acid has similar structural or chemical properties, e.g., replacement of leucine with isoleucine. A variant may have “nonconservative” changes, e.g., replacement of a glycine with a tryptophan. Analogous minor variations may also include amino acid deletions or insertions, or both. Guidance in determining which amino acid residues may be substituted, inserted, or deleted may be found using computer programs well known in the art such as Vector NTI Suite (InforMax, MD) software, CLC Genomics Workbench (Germantown, Md.) or EMBL-EBI online software.

Vine Maturity. Vine maturity refers to a plant's ability to continue to utilize carbohydrates and photosynthesize. Vine maturity is scored on a scale of 1 to 5 where 1=dead vines and 5=vines green, still flowering.

### Overview

The present disclosure relates to T-DNA characterized by a plurality of expression cassettes, transformation vectors comprising the T-DNA, and plants transformed therefrom. The transformed plants are characterized in that they contain the T-DNA insert region comprising the expression cassettes and exhibit a corresponding phenotype. The present disclosure also provides methods for identifying genetic material in transformed plants, including in food products made from such plants. The disclosure further relates to the materials and/or means for detecting plant transformation events and methods for detecting presence of plant transformation events

The T-DNA of the present disclosure addresses the need of the potato industry to improve the agronomic characteristics and nutritional value of potatoes. The incorporation of these desirable traits into existing potato varieties is difficult to achieve through traditional breeding because potato is tetraploid, highly heterozygous and sensitive to inbreeding depression.

The T-DNA of the present disclosure provides expression cassettes for reducing the tendency for black spot bruising; lowering reducing sugars; and/or conferring protection to potato virus Y (PVY); as well as by increasing the expression of resistance genes for conferring tolerance/resistance to potato late blight. Additional desirable traits include reduced formation of the acrylamide precursor asparagine with consequent decrease in accumulation of toxic Maillard products, including acrylamide, and improved quality and food color control.

### T-DNA

In some embodiments, the T-DNA of the present disclosure are native to the potato plant genome and do not contain any Agrobacterium DNA. In some embodiments, the T-DNA may comprise six, seven, eight, nine, ten or more cassettes and is inserted into a transformation vector.

T-DNA refers to a transfer DNA molecule that is capable of integrating into a plant genome. In some embodiments, the integration is via an Agrobacterium mediated transformation method. In some embodiments, the ends of the T-DNA molecule in the present disclosure are flanked by minimal sequences required for transfer (i.e., the right and the left T-DNA border sequences) that can be recognized by vir endonuclease proteins. The border regions commonly used in DNA constructs are designed for transferring transgenes into plants and comprise a nick site where an endonuclease digests the DNA to provide a site for insertion into the genome of a plant. In some embodiments, the T-DNA molecule contains a plurality of expression cassettes. The expression cassettes may express polynucleotide sequences for protein expression or downregulation of target genes. The T-DNAs of the present disclosure between the right and left border sequences are randomly inserted into a host genome anywhere flanked by target sites for a site-specific recombinase. The T-DNA may contain deletions, substitutions and/or additional insertions of DNA other than the target sites.

In some embodiments, the T-DNA comprises a ‘border region’, either a right T-DNA border (RB) also referred to as ‘right border’ or left T-DNA border (LB) also referred to as ‘left border’. Such border regions may comprises a core sequence flanked by a border inner region as part of the T-DNA flanking the border and/or a border outer region as part of the vector backbone flanking the border. The core sequences comprise about 22 bp in case of octopine-type vectors and about 25 bp in case of nopaline-type vectors. The core sequences in the right border region and left border region form imperfect repeats. Border core sequences are indispensable for recognition and processing by the Agrobacterium nicking complex consisting of at least VirD1 and VirD2. Core sequences flanking a T-DNA are sufficient to promote transfer of the T-DNA.

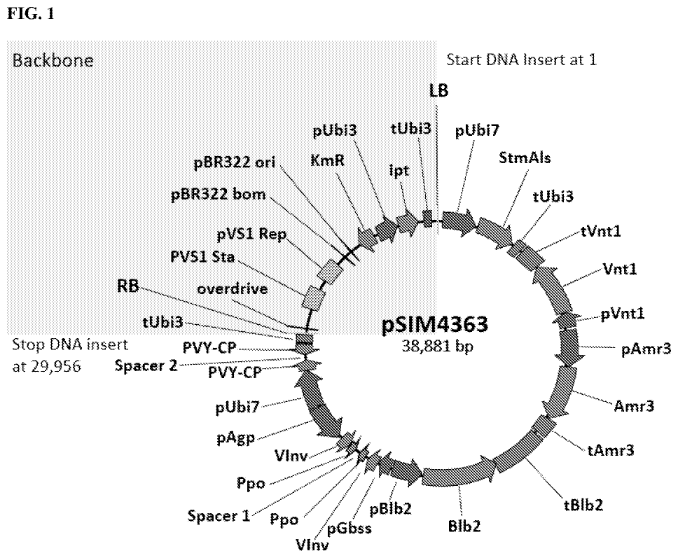

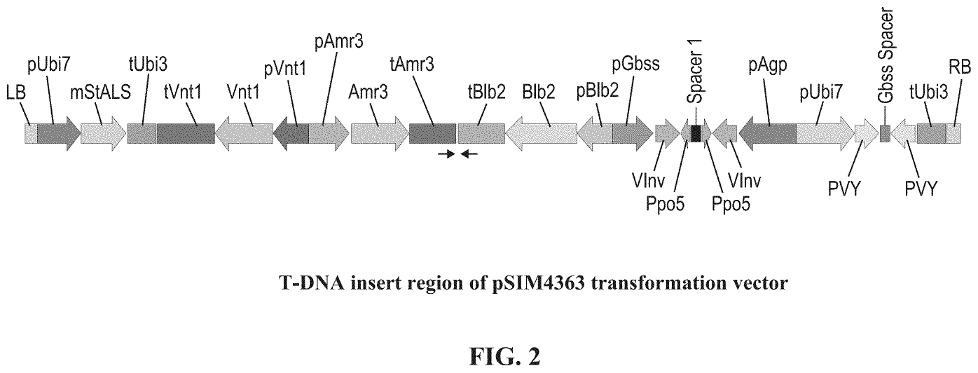

Shown in FIG. 1 and FIG. 2 is an example binary vector, pSIM4363 (FIG. 1) and T-DNA insert region (FIG. 2 and SEQ ID NO:2). The T-DNA of pSIM4363 has six expression cassettes, which comprise (i) a first cassette comprising a modified acetolactate synthase gene (StmAls) that encodes a modified ALS protein with at least one amino acid modification, as a selection marker; (ii) a second cassette comprising a resistance (R) gene to potato late blight caused by Phytophthora infestans (Pi), which is Rpi-Vnt1; (iii) a third cassette comprising a resistance (R) gene to potato late blight caused by Phytophthora infestans (Pi), which is Rpi-Amr3; and (iv) a fourth cassette comprising a resistance (R) gene to potato late blight caused by Phytophthora infestans (Pi), which is Rpi-Blb2. The T-DNA further comprises two expression cassettes having silencing elements, which comprises (i) a first cassette comprising an inverted repeat of two genes (i.e., vacuolar invertase (VInv) and polyphenol oxidase (Ppo)) facing each other; and (ii) a second cassette comprising an inverted repeat of a single gene (i.e., Potato virus Y coat protein; PVY-Cp) facing each other.

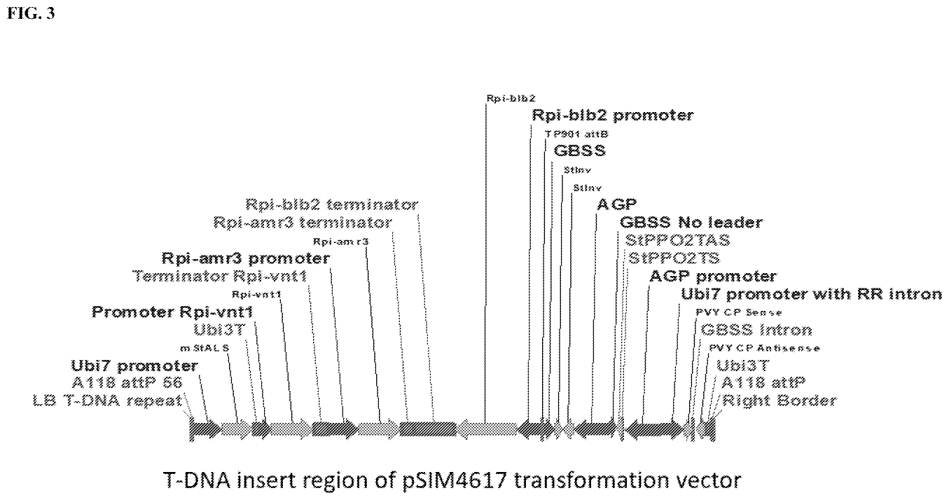

Sown in FIG. 3 is an example T-DNA insert region which comprises seven expression cassettes; (i) a first cassette comprising a modified acetolactate synthase gene (StmAls) that encodes a modified ALS protein with at least one amino acid modification, as a selection marker; (ii) a second cassette comprising a resistance (R) gene to potato late blight caused by Phytophthora infestans (Pi), which is Rpi-Vnt1; (iii) a third cassette comprising a resistance (R) gene to potato late blight caused by Phytophthora infestans (Pi), which is Rpi-Amr3; (iv) a fourth cassette comprising a resistance (R) gene to potato late blight caused by Phytophthora infestans (Pi), which is Rpi-Blb2. The T-DNA further comprises three expression cassettes having silencing elements, which comprises (i) a first cassette comprising an inverted repeat of a single gene (i.e., VInv) facing each other; (ii) a second cassette comprising an inverted repeat of a single gene (i.e., Ppo) facing each other; and (iii) a second cassette comprising an inverted repeat of a single gene (i.e., PVY-Cp) facing each other.

In the examples shown in FIG. 2-3 the function of the silencing elements is to silence a vacuolar invertase (VInv) for lowering reducing sugars (such as glucose and fructose), to silence a polyphenol oxidase gene (Ppo) for reduced black spot; and a to silence a potato virus Y coat protein-encoding gene (PVY-Cp), conferring protection to potato virus Y. Targeted gene silencing reduces the level of the RNA transcripts of the targeted genes in the tubers of the potato events by means of RNA interference (RNAi). In general, the inserted DNA contains silencing elements that, when expressed, generate variably-sized and unprocessed transcripts. These transcripts trigger the degradation of mRNAs that would normally code for an enzyme/protein. This results in much reduced levels of the targeted “silenced” enzymes/proteins.

In some embodiments, each expression cassette in the T-DNA insert region contains a single gene of interest (i.e., StmAls, Rpi-Vnt1, Rpi-Amr3, Rpi-B1b2, etc.) regulated by at least one promoter. In some embodiments, the expression cassette further comprises a terminator. In the examples described herein, the StmAls gene expression is controlled by a Ubi7 promoter (pUbi7) and a Ubi3 terminator (tUbi3) and the expression of each Rpi-Vnt1, Rpi-Amr3, and Rpi-Blb2 gene is regulated by its own native promoter (pRpi-Vnt1, pRpi-Amr3, pRpi-B1b2) and terminator (tRpi-Vnt1, tRpi-Amr3, tRpi-B1b2).

In other embodiments, a first silencing element in the T-DNA insert region contains an inverted repeat of two genes (i.e., VInv and Ppo) facing each other, but separated by spacer 1. The VInv and Ppo genes are inserted as inverted repeats between two convergent promoters, indicated as pGbss and pAgp, that are predominantly active in potato tubers. Plants containing the resulting silencing elements produce a diverse and unpolyadenylated array of RNA molecules in plant cells that dynamically and vigorously silence the intended target genes. The size of the RNA molecules was generally smaller than the distance between the two promoters employed because convergent transcription results in collisional transcription. A second silencing element in the pSIM4363 T-DNA insert region contains an inverted repeat of a single gene (i.e., PVY-Cp) facing each other, but separated by spacer 2. This inverted repeat of the PVY-Cp gene is controlled by a Ubi7 promoter (pUbi7) and a Ubi3 terminator (tUbi3).

In other embodiments, a first silencing element contains an inverted repeat of potato VInv gene (sense and antisense strands) facing each other to form a double-stranded RNA when being transcribed. A second silencing element contains an inverted repeat of potato Ppo gene (sense and antisense strands) facing each other to form a double-stranded RNA when being transcribed. Both VInv and Ppo genes are inserted as inverted repeats (sense and antisense strands), respectively, between two convergent promoters, indicated as pGbss and pAgp, that are predominantly active in potato tubers. Plants containing the resulting silencing elements produce a diverse and unpolyadenylated array of RNA molecules in plant cells that dynamically and vigorously silence the intended target genes. The size of the RNA molecules was generally smaller than the distance between the two promoters employed because convergent transcription results in collisional transcription. A third silencing element contains an inverted repeat of PVY-Cp gene facing each other for RNAi. This inverted repeat of the PVY-Cp gene is controlled by a Ubi7 promoter (pUbi7) and a Ubi3 terminator (tUbi3).

In some embodiments, the cassettes within the T-DNA contain no foreign DNA, and consists of DNA only from either the selected plant species or from a plant that is sexually compatible with the selected plant species.

Thus, in some embodiments, the tubers of the potato events described herein incorporate highly desirable traits, and are valuable in the potato industry and food market, as their tubers produce the desired traits and do not comprise transgenes.

In some embodiments, the cassettes within the T-DNA contain transgenes and/or genetic elements from other species. In some embodiments, the T-DNA insert may contain foreign DNA such as intervening sequence between cassettes.

In some embodiments, a silencing element can comprise the interfering RNA, a precursor to the interfering RNA, a template for the transcription of an interfering RNA or a template for the transcription of a precursor interfering RNA, wherein the precursor is processed within the cell to produce an interfering RNA. Thus, for example, a dsRNA silencing element includes a dsRNA molecule, a transcript or polyribonucleotide capable of forming a dsRNA, more than one transcript or polyribonucleotide capable of forming a dsRNA, a DNA encoding dsRNA molecule, or a DNA encoding one strand of a dsRNA molecule. When the silencing element comprises a DNA molecule encoding an interfering RNA, it is recognized that the DNA can be transiently expressed in a cell or stably incorporated into the genome of the cell.

The silencing element can reduce or eliminate the expression level of a target sequence by influencing the level of the target RNA transcript, by influencing translation and thereby affecting the level of the encoded polypeptide, or by influencing expression at the pre-transcriptional level (i.e., via the modulation of chromatin structure, methylation pattern, etc., to alter gene expression). See, e.g., Verdel et al. (2004) Science 303:672-676; Pal-Bhadra et al. (2004) Science 303:669-672; Allshire (2002) Science 297:1818-1819; Volpe et al. (2002) Science 297:1833-1837; Jenuwein (2002) Science 297:2215-2218; and Hall et al. (2002) Science 297:2232-2237.

### Traits

The insertion of desirable traits into the genome of potato plants presents difficulties because potato is tetraploid, highly heterozygous, and sensitive to in-breeding depression. It is therefore very difficult to efficiently develop potato plants having multiple desirable traits, such as reduced acrylamide, reduced black spot, and resistance or tolerance to pests and pathogens.

### Reduced Acrylamide

It has been very difficult to efficiently develop potato plants that produce less acrylamide and less harmful Maillard-reaction products, including N-Nitroso-N-(3-keto-1,2-butanediol)-3′-nitrotyramine (Wang et al., Arch Toxicol 70: 10-5, 1995), 5-hydroxymethyl-2-furfural (Janzowski et al., Food Chem Toxicol 38: 801-9, 2000), and other Maillard reaction products with mutagenic properties (Shibamoto, Prog Clin Biol Res 304: 359-76, 1989), during processing using conventional breeding.

Several methods have been tested and research is ongoing to reduce acrylamide through process changes, reduction in dextrose, and additives such as asparaginase, citrate, and competing amino acids. The required capital expense to implement process changes throughout the potato industry would cost millions of dollars. In addition to the expense, these process changes have significant drawbacks including potentially negative flavors associated with additives such as asparaginase or citrate. Typically, fry manufacturers add dextrose during processing of French fries to develop the desired golden brown color, but dextrose also increases the formation of acrylamide through the Maillard reaction. Significant reductions in acrylamide occur by merely omitting dextrose from the process; however, the signature golden brown colors must then be developed some other way (such as though the addition of colors like annatto). The use of alternate colors results in an absence of the typical flavors that develop through those browning reactions. Another challenge with the use of additives to reduce reactants like asparagine is moisture migration that occurs during frozen storage with the resulting return of asparagine to the surface and increased acrylamide. Finally, the blackening that occurs after potatoes are bruised affects quality and recovery in processing French fries and chips. Damaged and bruised potatoes must be trimmed or are rejected before processing, resulting in quality challenges or economic loss.

The present disclosure provides a reduced cold-induced sweetening (CIS). CIS is a phenomenon by which starch is converted to the simple reducing sugars, glucose and fructose, during cold storage. Upon processing at high temperatures, the glucose/fructose can interact with free amino acids in a Maillard reaction, which results in bitter, dark-pigmented products that may have increased levels of acrylamide—a suspected neurotoxin/carcinogen.

Down-regulating genes involved in the biosynthesis of acrylamide precursors using bioengineering provides a means to significantly reduce acrylamide in finished potato products.

The present disclosure teaches prevention of the accumulation of reducing sugars in the tuber by silencing vacuolar invertase (VInv) or water dikinase (R1). Lowering the levels of tuber asparagine by downregulation of asparagine synthase (Asn) results in significant reductions in acrylamide in French fires. Combining methods to reduce both reactants has an additive benefit for acrylamide reduction without negative consequences on taste and appearance of the final product.

The vacuolar invertase (VInv) gene hydrolyzes the sucrose produced from starch breakdown into one molecule of glucose and one of fructose. Thus, CIS can be minimized by reducing the expression of VInv. Reduced VInv expression can be obtained using a RNAi process or a sequence-specific nuclease to make a targeted mutation or knockout the VInv gene.

In some embodiments, vacuolar invertase activity is repressed, suppressed, knocked down or downregulated by reducing expression of VInv gene. In some embodiments, the silencing element for conferring resistance to CIS is an inverted repeat of vacuolar invertase (VInv) gene fragment.

To negatively impact on acrylamide formation of fried potato products, the present disclosure teaches reduced contents of the acrylamide precursors (i.e. asparagine) by silencing asparagine synthetase genes such as Asn1 and Asn2. Reduced Asn1 and/or Asn2 expression can be obtained using a RNAi process or a sequence-specific nuclease to make a targeted mutation or knockout the asparagine synthetase genes.

In some embodiments, asparagine synthetase activity is repressed, suppressed, knocked down or downregulated by reducing expression of Asn1 and/or Asn2 genes. In some embodiments, the silencing element for conferring resistance to CIS is an inverted repeat of Asn1 or Asn2 gene fragment.

Also, the present disclosure provides the genes targeted by RNAi to reduce the production of acrylamide during cooking include water dikinase (R1) and phosphorylase-L (PhL). In some embodiments, water dikinase and phosphorylase-L activities are repressed, suppressed, knocked down or downregulated by reducing expression of R1 and PhL genes. In some embodiments, the silencing element for conferring resistance to CIS is an inverted repeat of an inverted repeat of phosphorylase-L (PhL) gene fragment or an inverted repeat of kinase-R1 gene fragment.

### Decreased Black Spot Bruising

Black spot bruise occurs due to physical impact or following damage to tubers and can cause major losses to commercial potato processors that produce potato chips and French fries. Mechanical damage initiates enzymatic browning, with symptoms including production of black, brown and red pigments.

The reaction leading to pigment production is catalyzed by PPO, which converts monophenols to o-diphenols and o-dihydroxyphenols to o-quinones (Vamos-Vigyazo, CRC Critical Reviews in Food Science and Nutrition 14:44, 1981). PPO in potato is encoded by a gene family of at least six genes, including POTP1 and POTP2 (Hunt et al., Plant Mol Biol 21:59-68, 1993), as well as POT32, POT33, POT41 and POT72 (Thygesen et al., Plant Physiology 109:525-531, 1995).

Bruise resistance is a trait important to growers and processors alike, as reduced bruise damage can minimize crop rejection and waste in processing due to discarding of blackened fries and chips. The present disclosure provides decreased black spot bruising by reducing expression of tuber-specific polyphenol oxidase (PPO). This allows the tubers to tolerate physical impact without the subsequent enzymatic browning. Reduction of tuber-specific PPO can be obtained using a RNAi process or a sequence-specific nuclease to make a targeted mutation or knockout the PPO genes.

In some embodiments, PPO activity is repressed, suppressed, or downregulated by reducing expression of PPO genes encoding, such as, POTP1, POTP2, POT32, POT33, POT41 and POT72.

As used herein, “tolerance” or “tolerate” means a plant can be affected by physical impact or infected by pest, pathogen, or diseases, but is able to maintain or thrive a marketable yield and product value despite the physical impact or infection.

### Resistance to Potato Late Blight

Potato late blight disease, infamous for its implication in the Irish potato famine of the 1840s, is caused by infection of potato plants (Solanum tuberosum L.), and other solanaceous crops such as tomato and eggplant, by the pathogenic oomycete (water mold) Phytophthora infestans (Mont.) de Bary. The infection is characterized by black/brown lesions on the stems and leaves of the plant, which expand rapidly and become necrotic. Harvested afflicted potato tubers can decay upon storage or, if they survive the winter in storage or in the soil, can spread the disease to the next year's crop.

Because P. infestans is capable of acquiring resistance, efforts have been directed toward the identification of additional late blight resistance genes in wild potato species that are naturally resistant to P. infestans. For example, Rpi1, a late blight resistance gene from Solanum pinnatisectum, was described and mapped by Kuhl et al., 2001, Mol. Genet. Genomics 265: 977-985.

An example resistance gene described herein is one which controls resistance to the late blight caused by P. infestans. Such a gene may encode a polypeptide capable of recognizing and activating a defense response in a plant in response to challenge with said pathogen or an elicitor thereof. In some embodiments, a resistance (R) gene to potato late blight caused by P. infestans is Rpi-Vnt1, Rpi-Amr3 Rpi-Blb2, RpiBlb1, Rpi-mcq1, Rpi-Amr1, or Rpi-chc1. Additional resistance genes to potato late blight are well known in the art, see for example, Du and Vleeshouwers (2017) New Strategies Towards Durable Late Blight Resistance in Potato in Pages 161-169 of Book titled “The Potato Genome”, Springer; and Witek et al. (2021) A complex resistance locus in Solanum americanum recognizes a conserved Phytophthora effector. Nature Plants 7: 198-208; Zhu et al. (2012) Functional stacking of three resistance genes against Phytophthora infestans in potato. Transgenic Res. 21(1):89-99.

In some embodiments, a resistance (R) gene to potato late blight caused by P. infestans is Rpi-Vnt1. In some embodiments, a resistance (R) gene to potato late blight caused by P. infestans is Rpi-Amr3. In some embodiments, a resistance (R) gene to potato late blight caused by P. infestans is Rpi-B1b2.

As used herein, “resistance” or “resistant” means a plant that is immune to infection from pests, pathogens, or diseases, and/or infection never takes hold. It may also describe a plant that shows fewer or reduced symptoms to a pest or pathogen than a susceptible plant to that pest or pathogen. These terms are variously used to describe plants that show no symptoms as well as plants showing some symptoms but that are still able to produce marketable product with an acceptable yield. Some plants referred to as resistant are only so in the sense that they may still produce a crop or fruit, even though the plants may appear visually stunted and the yield is reduced compared to uninfected plants.

“Immunity” refers to a form of resistance characterized by absence of pest/pathogen replication even when the pest/pathogen is actively transferred into cells.

### Resistance to Potato Virus Y

Potato virus Y is a member of the Potyviridae family of viruses, the largest known group (family) of plant viruses. This family, in turn, is comprised of a number of genera, including genius Potyvirus (named after its type member, potato virus Y), genus Baymovirus (type member: barley yellow mosaic virus), and genus Ryemovirus (type member: ryegrass mosaic virus). See generally, Barnett, 0. W., Archives of Virology, 118:139-141 (1991); and Reichmnann et al., J. General Virology, 73:1-16 (1992).

Potato virus Y (PVY) is a potyvirus of global relevance to potato culture. Natural mutations in translation initiation factor eIF4E confer resistance to potyviruses in many plant species including potato. Transgenic expression of variants of the pvr1(2) gene, an eIF4E orthologue from pepper, confers resistance to Potato virus Y (PVY) in potato (Cavatorta J. et al, Plant Biotechnol J. 9(9):1014-1021; and Gutierrez Sanchez, P. A. et al., BMC Genomics 21, 18 (2020)). Also, Ryₛₜₒ, from the wild relative S. stoloniferum, confers extreme resistance to PVY and related viruses and is a valuable trait that is widely employed in potato resistance breeding programs. The Ryₛₜₒ gene encodes a nucleotide-binding leucine-rich repeat (NLR) protein with an N-terminal TIR domain. Its ectopic expression is sufficient for PVY perception and extreme resistance in transgenic potato plants (Baran, M. G. et al., Plant Biotechnology Journal 18(3):655-667 (2019).

In some embodiments, overexpression of eIF4E can confer virus resistance including resistance to PVY in a plant. In some embodiments, Ryₛₜₒ gene expression can confer resistance to PVY and related viruses in a plant.

In some embodiments, the present disclosure teaches expression of dsRNA targeting viral coat protein gene of Potato virus Y (PVY) using a RNAi pathway. In some embodiments, an inverted repeat of the PVY coat protein gene is present in the T-DNA insert region of the transformation vector taught herein, which can confer resistance to plant virus including Potato virus Y.

### Resistance to Other Pests and Pathogens

As will be understood by one skilled in the art, additional expression cassettes may be stacked in the T-DNA disclosed here to confer tolerance or resistance to other pests and pathogens, including, but not limited to, Potato virus X, Potato leaf roll virus, tobacco rattle virus, verticillium wilts, and pests such as plant-parasitic nematodes.

Protection from virus such as Potato Virus X and Potato leaf roll virus is achievable employing RNA interference to viral coat protein (CP) sequences. Potato plants engineered to express RNAi constructs to block the production of PVY CP are protected from infection and those expressing RNAi constructs to block the production of Potato leaf roll virus (PLRV) CP were resistant to PLRV (Kaniewski W. K. et al., AgBioForum, 7(1&2): 41-46 (2004)). The present disclosure teaches that RNAi constructs to several viruses could be stacked for robust virus resistance in plants.

Verticillium wilts are vascular wilt diseases caused by soil-borne fungal pathogens that belong to the Verticillium genus. Verticillium dahliae is the most notorious species and can infect hundreds of dicotyledonous hosts including potato. The Vel locus that confers race-specific resistance against Verticillium has been characterized and shown to be effective to resist race 1 isolates of V. dahliae (see U.S. Pat. No. 6,608,245 and Song Y et al., Molecular Plant Pathology 18(2): 195-2009 (2017)). The present disclosure teaches expression of the StVe1 gene for resistance to Verticillium dahlia in plants.

Bacillus thuringiensis (Bt) crystal proteins are pore-forming toxins used as insecticides around the world. Effective use of Bt (or Cry) proteins for Colorado potato beetle resistance was successfully deployed in potato (Kaniewski W. K. et al., AgBioForum, 7(1&2): 41-46 (2004)). The Cry proteins may target the invertebrate phylum Nematoda in several crops (Wei J. Z. et al, PNAS 100(5): 2760-2765 (2003); and Li X. Q. et al. Biological Control 47: 97-102 (2008)). The present disclosure provides that Cry proteins can be potential control agents of plant-parasitic nematodes in plants.

### Plant Breeding

Monocotyledon (monocot) refers to a flowering plant whose embryos have one cotyledon or seed leaf. Examples of monocots include, but are not limited to turf grass, maize, rice, oat, wheat, barley, sorghum, sugarcane, banana, orchid, iris, lily, onion, turf grass, and palm.

Dicotyledon (dicot) refers to a flowering plant whose embryos have two seed leaves or cotyledons. Examples of dicots include, but are not limited to, Arabidopsis, avocado, pepper, sugarbeet, broccoli, cassaya, cotton, poinsettia, tobacco, tomato, potato, sweet potato, cassava, legumes, alfalfa, lima bean, pea, chick pea, and soybean, eggplant, cucumber, grape, brassica, carrot, strawberry, lettuce, oak, maple, walnut, rose, mint, squash, daisy, and cactus.

The disclosure has use over a broad range of plants, monocots and dicots, including species from the genera Asparagus, Atropa, Avena, Brassica, Citrus, Citrullus, Capsicum, Cucumis, Cucurbita, Daucus, Fragaria, Glycine, Gossypium, Helianthus, Heterocallis, Hordeum, Hyoscyamus, Lactuca, Linum, Lolium, Lycopersicon, Malus, Manihot, Majorana, Medicago, Nicotiana, Oryza, Panieum, Pannesetum, Persea, Pisum, Pyrus, Prunus, Raphanus, Secale, Senecio, Sinapis, Solanum, Sorghum, Trigonella, Triticum, Vitis, Vigna, and Zea. Examples include tobacco and Arabidopsis, cereal crops such as maize, wheat, rice, soybean barley, rye, oats, sorghum, alfalfa, clover and the like, oil-producing plants such as canola, safflower, sunflower, peanut and the like, vegetable crops such as tomato tomatillo, potato, pepper, eggplant, sugar beet, carrot, cucumber, lettuce, pea and the like, horticultural plants such as aster, begonia, chrysanthemum, delphinium, zinnia, lawn and turfgrasses and the like.

The research leading to potato varieties which combine the advantageous characteristics referred to above is largely empirical. This research requires large investments of time, labor, and money. The development of a potato cultivar can often take a long time (up to eight years or more) from greenhouse to commercial usage. Breeding begins with careful selection of superior parents to incorporate the most important characteristics into the progeny. Since all desired traits usually do not appear with just one cross, breeding must be cumulative.

Present breeding techniques continue with the controlled pollination of parental clones. Typically, pollen is collected in gelatin capsules for later use in pollinating the female parents. Hybrid seeds are sown in greenhouses and tubers are harvested and retained from thousands of individual seedlings. The next year one to four tubers from each resulting seedling are planted in the field, where extreme caution is exercised to avoid the spread of virus and diseases. From this first-year seedling crop, several “seed” tubers from each hybrid individual which survived the selection process are retained for the next year's planting. After the second year, samples are taken for density measurements and fry tests to determine the suitability of the tubers for commercial usage. Plants which have survived the selection process to this point are then planted at an expanded volume the third year for a more comprehensive series of fry tests and density determinations. At the fourth-year stage of development, surviving selections are subjected to field trials in several states to determine their adaptability to different growing conditions. Eventually, the varieties having superior qualities are transferred to other farms and the seed increased to commercial scale. Generally, by this time, eight or more years of planting, harvesting and testing have been invested in attempting to develop the new and improved potato cultivars.

Backcrossing methods can be used with the present disclosure to improve or introduce a characteristic into the variety. The term “backcrossing” as used herein refers to the repeated crossing 1, 2, 3, 4, 5, 6, 7, 8, 9 or more times of a hybrid progeny back to the recurrent parents. The parental potato plant which contributes the gene(s) for the one or more desired characteristics is termed the nonrecurrent or donor parent. This terminology refers to the fact that the nonrecurrent parent is used one time in the backcross protocol and therefore does not recur. The parental potato plant to which the gene or genes from the nonrecurrent parent are transferred is known as the recurrent parent as it is used for several rounds in the backcrossing protocol. In a typical backcross protocol, the original variety of interest (recurrent parent) is crossed to a second variety (nonrecurrent parent) that carries the gene(s) of interest to be transferred. The resulting progeny from this cross are then crossed again to the recurrent parent and the process is repeated until a potato plant is obtained wherein essentially all of the desired morphological and physiological characteristics of the recurrent parent are recovered in the converted plant, in addition to the one or more genes transferred from the nonrecurrent parent.

The selection of a suitable recurrent parent is an important step for a successful backcrossing procedure. The goal of a backcross protocol is to alter or substitute one or more traits or characteristics in the original variety. To accomplish this, one or more genes of the recurrent variety are modified, substituted or supplemented with the desired gene(s) from the nonrecurrent parent, while retaining essentially all of the rest of the desired genes, and therefore the desired physiological and morphological constitution of the original variety. The choice of the particular nonrecurrent parent will depend on the purpose of the backcross. One of the major purposes is to add some commercially desirable, agronomically important trait to the plant. The exact backcrossing protocol will depend on the characteristic or trait being altered or added to determine an appropriate testing protocol. Although backcrossing methods are simplified when the characteristic being transferred is a dominant allele, a recessive allele may also be transferred. In this instance, it may be necessary to introduce a test of the progeny to determine if the desired characteristic has been successfully transferred.

### Molecular Techniques

With the advent of molecular biological techniques that have allowed the isolation and characterization of genes that encode specific protein products, scientists in the field of plant biology developed a strong interest in engineering the genome of plants to contain and express foreign genes, or additional, or modified versions of native, or endogenous, genes (perhaps driven by different promoters) in order to alter the traits of a plant in a specific manner. Such foreign additional and/or modified genes are referred to herein collectively as “transgenes”. Over the last fifteen to twenty years several methods for producing transgenic plants have been developed, and the present disclosure, in particular embodiments, also relates to transformed versions of the claimed variety or line.

Plant transformation involves the construction of an expression vector which will function in plant cells. Such a vector comprises DNA comprising a gene under control of, or operatively linked to, a regulatory element (for example, a promoter). The expression vector may contain one or more such operably linked gene/regulatory element combinations. The vector(s) may be in the form of a plasmid, and can be used alone or in combination with other plasmids, to provide transformed potato plants, using transformation methods as described below to incorporate transgenes into the genetic material of the potato plant(s).

Traditional plant breeding typically relies on the random recombination of plant chromosomes to create varieties that have new and improved characteristics. According to standard, well-known techniques, genetic “expression cassettes,” comprising genes and regulatory elements, are inserted within the borders of Agrobacterium-isolated transfer DNAs (“T-DNAs”) and integrated into plant genomes. Agrobacterium-mediated transfer of T-DNA material typically comprises the following standard procedures: (1) in vitro recombination of genetic elements, to produce an expression cassette for selection of transformation, (2) insertion of this expression cassette, often together with at least one other expression cassette into a T-DNA region of a binary vector, (3) transfer of the sequences located between the T-DNA borders, often accompanied with some or all of the additional binary vector sequences from Agrobacterium to the plant cell, and (4) selection of stably transformed plant cells that display a desired trait, such as an increase in yield, improved vigor, enhanced resistance to diseases and insects, or greater ability to survive under stress.

In some embodiments, genetic engineering methods may rely on the introduction of foreign, not-endogenous nucleic acids, including regulatory elements such as promoters and terminators, and genes that are involved in the expression of a new trait or function as markers for identification and selection of transformants, from viruses, bacteria and plants. Marker genes are typically derived from bacterial sources and confer antibiotic or herbicide resistance.

In the “anti-sense” technology, the sequence of native genes is inverted to silence the expression of the gene in transgenic plants.

The present disclosure provides a T-DNA insert region conferring desired traits taught herein. In some embodiments, the T-DNA insert region comprises a nucleic acid sequence set forth in SEQ ID NO:2 or SEQ ID NO:3.

In one embodiment, the present disclosure provides a nucleic acid sequence that share at least 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NOs 1-3.

Methods of alignment of sequences for comparison are well known in the art. Various programs and alignment algorithms are described in: Smith and Waterman (Adv. Appl. Math., 2:482, 1981); Needleman and Wunsch (J. Mol. Biol., 48:443, 1970); Pearson and Lipman (Proc. Natl. Acad. Sci., 85:2444, 1988); Higgins and Sharp (Gene, 73:237 44, 1988); Higgins and Sharp (CABIOS, 5:151 53, 1989); Corpet et al. (Nuc. Acids Res., 16:10881 90, 1988); Huang et al. (Comp. Appls Biosci., 8:155 65, 1992); and Pearson et al. (Meth. Mol. Biol., 24:307 31, 1994). Altschul et al. (Nature Genet., 6:119 29, 1994) presents a detailed consideration of sequence alignment methods and homology calculations.

### Transgene Stacking Using the GAANTRY System

The GAANTRY system (Gene Assembly in Agrobacterium by Nucleic acid Transfer using Recombinase technologY) leverages recombinase-mediated stacking technology. The specificity and efficiency of recombinases make them extremely attractive for genome engineering. Advancements in molecular biology and recombinases have paved the way for gene stacking with the assistance of unidirectional recombination systems. Development of this high-efficiency gene stacking system uses the specificity of the recombinases to effectively deliver the target genes of interest to a predetermined position. This is a flexible and effective system for stably stacking multiple genes within an Agrobacterium virulence plasmid Transfer-DNA (T-DNA).

Agrobacterium-mediated transformation of plants with one or a few genes is relatively routine, but the assembly and transformation of large constructs carrying multiple genes and their efficient use to generate high-quality transgenic plants has been a challenge.

The present disclosure teaches GAANTRY, which is a flexible and effective system for stably stacking multiple genes within an Agrobacterium virulence plasmid Transfer-DNA (T-DNA). This GAANTRY system is well described in Collier, R. et al (2018), Plant Journal 95, 573-583 and McCue et al (2019) BMC Research Notes 12, 457, each of which is incorporated herein by reference for all purposes.

The GAANTRY system is based on the combined use of unidirectional integration and excision controlled by three site-specific serine recombinases, which is an effective and stable system for stacking multiple genes within an Agrobacterium virulence plasmid T-DNA. The gene stacking system utilizes ‘P-Donor’ and ‘B-Donor’ cloning vectors, and ‘P-Helper’ and ‘B-Helper’ vectors, for the insertion of sequences of interest. The P-Donor and B-Donor vectors contain either attP or attB, respectively, recognition sites enabling precise integration into the virulence plasmid of the GAANTRY ArPORT1 strain. Plant transformation with T-DNA expressed from a GAANTRY modified Agrobacterium can produce high quality events that contain low copies of a complete T-DNA with limited incorporation of vector ‘backbone’ sequences (Collier, 2018).

The resulting engineered Agrobacterium strain can be directly used for plant transformation. The gene stacking strategy is efficient, precise, modular, and allows control over the orientation and order in which genes are stacked within the T-DNA.

In some embodiments, at least 2, at least 3, at least 4, at least 5, at least 6, at least 7, at least 8, at least 9, or at least 10 T-DNAs can be stacked in the transformation vector taught herein using the GAANTRY system.

In some embodiments, at least 2, at least 3, at least 4, at least 5, at least 6, at least 7, at least 8, at least 9, or at least 10 genes can be stacked in the T-DNA region taught herein using the GAANTRY system.

In some embodiments, at least 2, at least 3, at least 4, at least 5, at least 6, at least 7, at least 8, at least 9, or at least 10 genes can be stacked in the transformation vector taught herein using the GAANTRY system.

In some embodiments, at least 2, at least 3, at least 4, at least 5, at least 6, at least 7, at least 8, at least 9, or at least 10 expression cassettes for gene expression can be stacked in the T-DNA region taught herein using the GAANTRY system.

In some embodiments, at least 2, at least 3, at least 4, at least 5, at least 6, at least 7, at least 8, at least 9, or at least 10 expression cassettes for downregulation/silencing of target gene(s)can be stacked in the transformation vector taught herein using the GAANTRY system.

The present disclosure provide 2-stack T-DNA construct, 3-stack T-DNA construct, 4-stack T-DNA construct, 5-stack T-DNA construct, 6-stack T-DNA construct, 7-stack T-DNA construct, 8-stack T-DNA construct, 9-stack T-DNA construct, 10-stack T-DNA construct or more than 10-stack T-DNA construct.

### Expression Vectors for Potato Transformation

Examples of potato transformation vectors are well known in the art, such as for example, pSIM1278 and pSIM1678. Further details relating to these vector compositions and plant events selected from pSIM1278 and/or pSIM1678 transformants are found in U.S. Pat. Nos. 9,328,352, 9,873,885, 8,710,311, 8,754,303, 8,889,963, 9,918,441, 8,889,964, 9,909,141, 9,924,647, and 9,968,043, which are all incorporated herein by reference.

### Expression Vectors for Potato Transformation: Marker Genes

Expression vectors include at least one genetic marker, operably linked to a regulatory element (a promoter, for example) that allows transformed cells containing the marker to be either recovered by negative selection, i.e., inhibiting growth of cells that do not contain the selectable marker gene, or by positive selection, i.e., screening for the product encoded by the genetic marker. Many commonly used selectable marker genes for plant transformation are well known in the transformation arts, and include, for example, genes that code for enzymes that metabolically detoxify a selective chemical agent which may be an antibiotic or an herbicide, or genes that encode an altered target which is insensitive to the inhibitor. A few positive selection methods are also known in the art.

One commonly used selectable marker gene for plant transformation is the neomycin phosphotransferase II (nptII) gene which, when under the control of plant regulatory signals, confers resistance to kanamycin. Fraley et al., Proc. Natl. Acad. Sci. U.S.A., 80:4803 (1983). The aminoglycoside phosphotransferases APH(3′)II and APH(3′)I carried by transposons TnS and Tn601 respectively were shown to inactivate the related aminoglycoside antibiotics G418), neomycin and kanamycin (Davies and Smith, 1978; Jimenez and Davies, 1980). The kanamycin resistance (KmR) gene from Staphylococcus aureus, which are used for the present disclosure, was sequenced and identified when compared with similar genes isolated from Streptomyces fradiae and from two transposons, Tn5 and Tn903, originally isolated from Klebsiella pneumoniae and Salmonella typhimurium, respectively (Gray and Fitch, 1983). Another commonly used selectable marker gene is the hygromycin phosphotransferase gene which confers resistance to the antibiotic hygromycin. Vanden Elzen et al., Plant Mol. Biol., 5:299 (1985).

Additional selectable marker genes of bacterial origin that confer resistance to antibiotics include gentamycin acetyl transferase, streptomycin phosphotransferase and aminoglycoside-3′-adenyl transferase, the bleomycin resistance determinant. Hayford et al., Plant Physiol. 86:1216 (1988), Jones et al., Mol. Gen. Genet., 210:86 (1987), Svab et al., Plant Mol. Biol. 14:197 (1990) Hille et al., Plant Mol. Biol. 7:171 (1986). Other selectable marker genes confer resistance to herbicides such as glyphosate, glufosinate or bromoxynil. Comai et al., Nature 317:741-744 (1985), Gordon-Kamm et al., Plant Cell 2:603-618 (1990) and Stalker et al., Science 242:419-423 (1988).

Selectable marker genes for plant transformation not of bacterial origin include, for example, mouse dihydrofolate reductase, plant 5-enolpyruvylshikimate-3-phosphate synthase and plant acetolactate synthase. Eichholtz et al., Somatic Cell Mol. Genet. 13:67 (1987), Shah et al., Science 233:478 (1986), Charest et al., Plant Cell Rep. 8:643 (1990).

Another class of marker genes for plant transformation requires screening of presumptively transformed plant cells rather than direct genetic selection of transformed cells for resistance to a toxic substance such as an antibiotic. These genes are particularly useful to quantify or visualize the spatial pattern of expression of a gene in specific tissues and are frequently referred to as reporter genes because they can be fused to a gene or gene regulatory sequence for the investigation of gene expression. Commonly used genes for screening presumptively transformed cells include beta-glucuronidase (GUS), beta-galactosidase, luciferase and chloramphenicol acetyltransferase. Jefferson, R. A., Plant Mol. Biol. Rep. 5:387 (1987), Teeri et al., EMBO J. 8:343 (1989), Koncz et al., Proc. Natl. Acad. Sci. USA 84:131 (1987), DeBlock et al., EMBO J. 3:1681 (1984). In vivo methods for visualizing GUS activity that do not require destruction of plant tissue are available. Molecular Probes publication 2908, IMAGENE GREEN, p. 1-4 (1993) and Naleway et al., J. Cell Biol. 115:151a (1991). However, these in vivo methods for visualizing GUS activity have not proven useful for recovery of transformed cells because of low sensitivity, high fluorescent backgrounds and limitations associated with the use of luciferase genes as selectable markers.

In some aspects, a gene encoding Green Fluorescent Protein (GFP) has been utilized as a marker for gene expression in prokaryotic and eukaryotic cells. Chalfie et al., Science 263:802 (1994). GFP and mutants of GFP may be used as screenable markers.

### Expression Vectors for Potato Transformation: Promoters

Genes included in expression vectors are typically driven by a nucleotide sequence comprising a regulatory element, for example, a promoter. Several types of promoters are well known in the transformation arts as are other regulatory elements that can be used alone or in combination with promoters.

As used herein, “promoter” includes reference to a region of DNA upstream from the start of transcription and involved in recognition and binding of RNA polymerase and other proteins to initiate transcription. A “plant promoter” is a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred”. Promoters that initiate transcription only in a certain tissue are referred to as “tissue-specific”. A “cell-type” specific promoter primarily drives expression in certain cell types in one or more organs, for example, vascular cells in roots or leaves. An “inducible” promoter is a promoter which is under environmental control. Examples of environmental conditions that may affect transcription by inducible promoters include anaerobic conditions or the presence of light. Tissue-specific, tissue-preferred, cell type specific, and inducible promoters constitute the class of “non-constitutive” promoters. A “constitutive” promoter is a promoter that is active under most environmental conditions, and cell types.

In some embodiments, a polyubiquitin promoter (pUbi7) is operably linked to the StmAls gene to initiate transcription. In some embodiments, a polyubiquitin promoter (pUbi7) is operably linked to an inverted repeat of the PVY-Cp gene to initiate transcription. In other embodiments, a Rpi-Vnt1 promoter is operably linked to the Rpi-Vnt1 gene to initiate transcription. In other embodiments, a Rpi-Amr3 promoter is operably linked to the Rpi-Amr3 gene to initiate transcription. In other embodiments, a Rpi-Blb2 promoter is operably linked to the Rpi-Blb2 gene to initiate transcription.

### A. Inducible Promoters

An inducible promoter is operably linked to a gene for expression in a plant. Optionally, the inducible promoter is operably linked to a nucleotide sequence encoding a signal sequence which is operably linked to a gene for expression in a plant. With an inducible promoter the rate of transcription increases in response to an inducing agent.

Any inducible promoter can be used in the instant disclosure. See Ward et al., Plant Mol. Biol. 22:361-366 (1993). Exemplary inducible promoters include, but are not limited to, that from the ACEI system which responds to copper (Mett et al., PNAS 90:4567-4571 (1993)); In2 gene from maize which responds to benzenesulfonamide herbicide safeners (Hershey et al., Mol. Gen Genetics 227:229-237 (1991) and Gatz et al., Mol. Gen. Genetics 243:32-38 (1994)) or Tet repressor from Tn10 (Gatz et al., Mol. Gen. Genetics 227:229-237 (1991)). A particularly preferred inducible promoter is a promoter that responds to an inducing agent to which plants do not normally respond. An exemplary inducible promoter is the inducible promoter from a steroid hormone gene, the transcriptional activity of which is induced by a glucocorticosteroid hormone. Schena et al., Proc. Natl. Acad. Sci. USA 88:0421 (1991).

### B. Constitutive Promoters

A constitutive promoter is operably linked to a gene for expression in a plant or the constitutive promoter is operably linked to a nucleotide sequence encoding a signal sequence which is operably linked to a gene for expression in a plant.

Many different constitutive promoters can be utilized in the instant disclosure. Exemplary constitutive promoters include, but are not limited to, the promoters from plant viruses such as the 35S promoter from CaMV (Odell et al., Nature 313:810-812 (1985)) and the promoters from such genes as rice actin (McElroy et al., Plant Cell 2: 163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)) and maize H3 histone (Lepetit et al., Mol. Gen. Genetics 231:276-285 (1992) and Atanassova et al., Plant Journal 2 (3): 291-300 (1992)).

The ALS promoter, Xbal/Ncol fragment 5′ to the Brassica napus ALS3 structural gene (or a nucleotide sequence similarity to said Xbal/Ncol fragment), represents a particularly useful constitutive promoter. See PCT application WO 96/30530.

In some embodiments, the present disclosure teaches use of a potato ubiquitin promoter (pUbi7) for constitutive expression of a gene of interest. Isolation of a polyubiquitin promoter and its expression in transgenic potato plants is described in Garbarino et al., Plant Physiology 109(4):1371-1378 (1995), which is hereby incorporated by reference in its entirety.

### C. Tissue-Specific or Tissue-Preferred Promoters

A tissue-specific promoter is operably linked to a gene for expression in a plant. Optionally, the tissue-specific promoter is operably linked to a nucleotide sequence encoding a signal sequence which is operably linked to a gene for expression in a plant. Plants transformed with a gene of interest operably linked to a tissue-specific promoter produce the protein product of the transgene exclusively, or preferentially, in a specific tissue.

Any tissue-specific or tissue-preferred promoter can be utilized in the instant disclosure. Exemplary tissue-specific or tissue-preferred promoters include, but are not limited to, a root-preferred promoter—such as that from the phaseolin gene (Murai et al., Science 23:476-482 (1983) and Sengupta-Gopalan et al., Proc. Natl. Acad. Sci. USA 82:3320-3324 (1985)); a leaf-specific and light-induced promoter such as that from cab or rubisco (Simpson et al., EMBO J. 4(11):2723-2729 (1985) and Timko et al., Nature 318:579-582 (1985)); an anther-specific promoter such as that from LAT52 (Twell et al., Mol. Gen. Genetics 217:240-245 (1989)); a pollen-specific promoter such as that from Zm13 (Guerrero et al., Mol. Gen. Genetics 244:161-168 (1993)) or a microspore-preferred promoter such as that from apg (Twell et al., Sex. Plant Reprod. 6:217-224 (1993)).

### Expression Vectors for Potato Transformation: Terminators

As used herein, the term “terminator” or “termination sequence” generally refers to a 3′ flanking region of a gene that contains nucleotide sequences which regulate transcription termination and typically confer RNA stability.

The disclosure provides terminator sequences that find use in proper transcriptional processing of recombinant nucleic acids in the transformation vectors taught herein. Although terminator sequences do not by themselves initiate gene transcription, their presence can increase accurate processing and termination of the RNA transcript, and result in message stability. The use of recombinant terminator sequences is established in the art. It is appreciated that an understanding of the molecular mechanisms underlying terminator sequence activity are not required to make or use the present disclosure.

In some embodiments, a polyubiquitin terminator (tUbi3) is operably linked to the StmAls gene to stop transcription. In some embodiments, a polyubiquitin terminator (tUbi3) is operably linked to an inverted repeat of the PVY-Cp gene to stop transcription. In other embodiments, a Rpi-Vnt1 terminator is operably linked to the Rpi-Vnt1 gene to stop transcription. In other embodiments, a Rpi-Amr3 terminator is operably linked to the Rpi-Amr3 gene to stop transcription. In other embodiments, a Rpi-B1b2 terminator is operably linked to the Rpi-B1b2 gene to stop transcription.

### Taxonomy of the Genus Solanum

The Solanaceae family contains several well-known cultivated crops such as tomato (Solanum lycopersicum also referred to as Lycopersicon esculentum), eggplant (Solanum melogena), tobacco (Nicotiana tabacum), pepper (Capsicum annuum) and potato (Solanum tuberosum). Within the genus Solanum, over a thousand species have been recognized. Potatoes will not hybridize with non-tuber bearing Solanum (tomato, eggplant, etc.) species including weeds commonly found in and around commercial potato fields (Love 1994).

The genus Solanum is divided into several subsections, of which the subsection potatoe contains all tuber-bearing potatoes. The subsection potatoe is divided into series, of which tuberosa is relevant to this document. Within the series tuberosa approximately 54 species of wild and cultivated potatoes are found. One of these is S. tuberosum.

S. tuberosum is divided into two subspecies: tuberosum and andigena. The subspecies tuberosum is the cultivated potato widely in use as a crop plant in, for example, North America and Europe. The subspecies andigena is also a cultivated species, but cultivation is restricted to Central and South America (Hanneman 1994).

The only two wild potato species that grow within the borders of the USA, and for which specimens exist in gene banks, include the tetraploid species S. fendleri (recently reclassified as S. stoloniferum; however, some sources, including the Inter-genebank Potato Database, still use the S. fendleri designation) and the diploid species S. jamesii (Bamberg et al. 2003; IPD 2011; Bamberg and del Rio 2011a; Bamberg and del Rio 2011b; Spooner et al. 2004). Love (1994) reported that a third species, S. pinnatisectum, is also a native species in the USA. However, Spooner et al. (2004) determined that what was previously thought to be S. pinnatisectum was in fact S. jamesii. Through more than 10 years of field work and assessments of existing records, Bamberg et al. (2003) and Spooner et al. (2004) established the presence of only these two species, S. fendleri and S. jamesii, in the U.S. These researchers also attempted to verify previously recorded locations, and through this process, updated the maps of current known locations of these species, providing latitude and longitude locations for each documented population (Bamberg et al. 2003) and distribution maps (Spooner et al. 2004). These species mostly reside in dry forests, scrub desert, and sandy areas at altitudes of 5,000 to 10,000 feet, well isolated from most commercial production areas (Bamberg and del Rio 2011a).

While there is some overlap between the acreage used for commercial production and occurrence of wild species on a county level, the majority of the potato production in the United States is not in wild potato zones. However, there is a possibility that a few wild potato plants may be growing near potato fields (Love 1994). Spooner et al. (2004) describe S. jamesii habitat in the U.S. as among boulders on hillsides, sandy alluvial stream bottoms, in gravel along trails or roadways, rich organic soil of alluvial valleys, sandy fallow fields, grasslands, juniper-pinyon scrub deserts, oak thicket, coniferous and deciduous forests at elevations between 4,500 to 9,400 feet. They describe S. fendleri habitat similarly, and at elevations between 4700 to 11,200 feet.

With respect to potato plants, Solanum tuberosum ssp. tuberosum is an example of one of the most widely cultivated potato varieties, although there are thousands of potato varieties worldwide. Modern varieties of Solanum tuberosum are the most widely cultivated. There are two major subspecies of Solanum tuberosum: andigena, or Andean; and tuberosum, or Chilean. In general, well-known cultivated varieties include, but are not limited to, russets, reds, whites, yellows (also called Yukons) and purples. Popular varieties, also known as cultivars, include: Adirondack Blue, Adirondack Red, Agata, Almond, Amandine, Anya, Arran Victory, Atlantic, Bamberg, Belle de Fontenay, BF-15, Bildtstar, Bintje, Blue Congo, Bonnotte, Cabritas, Camota, Chelina, Chiloé, Cielo, Clavela Blanca, Désirée, Fianna, Fingerling, Flava, Golden Wonder, Innovator, Jersey Royal, Kerr's Pink, Kestrel, King Edward, Kipfler, Lady Balfour, Linda, Marfona, Maris Piper, Marquis, Nicola, Pachacoñ a, Pink Eye, Pink Fir Apple, Primura, Ratte, Red Norland, Red Pontiac, Rooster, Russet Burbank, Russet Norkotah, Selma, Shepody, Sieglinde, Sirco, Spunta, Stobrawa, Vivaldi, Vitelotte, Yellow Finn, and Yukon Gold. Any of these cultivated varieties may be transformed or modified as disclosed herein to positively or negatively expressing target gene(s) taught herein for conferring desired trait(s). A wild potato plant is not a cultivated potato plant variety. In some embodiments, the present disclosure uses Russet Burbank and/or Russet Norkotah for transformation of a plant transformation vector of the present disclosure.

The commercially valuable potato plant variety used in the present disclosure is Russet Burbank and/or Russet Norkotah TX 296 (NK). For the Russet Burbank, Luther Burbank developed this variety in the early 1870s. Plants are vigorous and have an indeterminate type of growth. Stems are thick, prominently angled and finely mottled. Leaflets are long to medium in width and light to medium green in color. The blossoms are few, white and not fertile. The cultivar is tolerant to common scab but is susceptible to Fusarium and Verticillium wilts, leafroll and net necrosis, and viruses. Plants require conditions of high and uniform soil moisture and controlled nitrogen fertility to produce tubers free from knobs, pointed ends and dumbbells. Jelly-end and sugar-end develop in tubers when plants are subjected to stress. The tubers produced are large brown-skinned and white-fleshed, display good long-term storage characteristics, and represent the standard for excellent baking and processing quality. Russet Burbank varieties have a high susceptibility to develop black spot bruise and also have high free asparagine content and high senescence sweetening potential (Am. J. Potato Res (1966) 43: 305-314). The variety is sterile and widely grown in the Northwest and Midwest, especially for the production of French fries.

In some embodiments, the present disclosure relates to plants comprising the T-DNA described herein. In some embodiments, the disclosure relates to plants transformed with vector pSIM4363 or pSIM4617. In some embodiments, the plants comprising the T-DNA described herein exhibit resistance to potato late blight caused by Phytophthora infestans; decreased black spot bruising; decreased reducing sugars; and resistance to potato virus Y.

Methods of modifying nucleic acid constructs to increase expression levels in plants are also generally known in the art (see, e.g. Rogers et al., 260 J. Biol. Chem. 3731-38, 1985; Cornejo et al., 23 Plant Mol. Biol. 567: 81,1993). In engineering a plant system to affect the rate of transcription of a protein, various factors known in the art, including regulatory sequences such as positively or negatively acting sequences, enhancers and silencers, as well as chromatin structure may have an impact. The present disclosure provides that at least one of these factors may be utilized in engineering plants to express a protein of interest. The regulatory sequences of the present disclosure are native genetic elements, i.e., are isolated from the selected plant species to be modified.

### Methods for Potato Transformation

Numerous methods for plant transformation have been developed and are well known in the art, including biological and physical plant transformation protocols. See, for example, Miki et al., “Procedures for Introducing Foreign DNA into Plants” in Methods in Plant Molecular Biology and Biotechnology, Glick, B. R. and Thompson, J. E. Eds. (CRC Press, Inc. Boca Raton, 1993) pages 67-88. In addition, expression vectors and in-vitro culture methods for plant cell or tissue transformation and regeneration of plants are available. See, for example, Gruber et al., “Vectors for Plant Transformation” in Methods in Plant Molecular Biology and Biotechnology, Glick, B. R. and Thompson, J. E. Eds. (CRC Press, Inc., Boca Raton, 1993) pages 89-119.

### A. Agrobacterium-Mediated Transformation

One method for introducing an expression vector into plants is based on the natural transformation system of Agrobacterium. See, for example, Horsch et al., Science 227:1229 (1985). A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant. See, for example, Kado, C. I., Crit. Rev. Plant Sci. 10:1 (1991). Descriptions of Agrobacterium vector systems and methods for Agrobacterium-mediated gene transfer are provided by Gruber et al., supra, Miki et al., supra and Moloney et al., Plant Cell Reports 8:238 (1989). See also, U.S. Pat. No. 5,563,055 (Townsend and Thomas), issued Oct. 8, 1996. There are numerous patents governing Agrobacterium mediated transformation and particular DNA delivery plasmids designed specifically for use with Agrobacterium—for example, U.S. Pat. No. 4,536,475, EP0265556, EP0270822, WO8504899, WO8603516, U.S. Pat. No. 5,591,616, EP0604662, EP0672752, WO8603776, WO9209696, WO9419930, WO9967357, U.S. Pat. No. 4,399,216, WO8303259, U.S. Pat. No. 5,731,179, EP068730, WO9516031, U.S. Pat. Nos. 5,693,512, 6,051,757 and EP904362A1, which are all hereby incorporated by reference in their entirety.

Agrobacterium-mediated plant transformation involves as a first step the placement of DNA fragments cloned on plasmids into living Agrobacterium cells, which are then subsequently used for transformation into individual plant cells. Agrobacterium-mediated plant transformation is thus an indirect plant transformation method. Alternatively, Agrobacterium-mediated plant transformation involves cloning DNA fragments into the disarmed Ti or Vi plasmid of Agrobacterium, such as described in Collier (2018), and using the resulting engineered Agrobacterium for plant transformation.

Agrobacterium-mediated transformation is achieved through the use of a genetically engineered soil bacterium belonging to the genus Agrobacterium. Several Agrobacterium species mediate the transfer of a specific DNA known as “T-DNA” that can be genetically engineered to carry any desired piece of DNA into many plant species. The major events marking the process of T-DNA mediated pathogenesis are: induction of virulence genes, processing and transfer of T-DNA. This process is the subject of many reviews (Ream, 1989; Howard and Citovsky, 1990; Kado, 1991; Hooykaas and Schilperoort, 1992; Winnans, 1992; Zambryski, 1992; Gelvin, 1993; Binns and Howitz, 1994; Hooykaas and Beijersbergen 1994; Lessl and Lanka, 1994; Zupan and Zambryski, 1995).

Agrobacterium-mediated genetic transformation of plants involves several steps. The first step, in which the Agrobacterium and plant cells are first brought into contact with each other, is generally called “inoculation”. Following the inoculation step, the Agrobacterium and plant cells/tissues are usually grown together for a period of several hours to several days or more under conditions suitable for growth and T-DNA transfer. This step is termed “co-culture”. Following co-culture and T-DNA delivery, the plant cells are often treated with bacteriocidal and-or bacteriostatic agents to kill the Agrobacterium.

Although transgenic plants produced through Agrobacterium-mediated transformation generally contain a simple integration pattern as compared to microparticle-mediated genetic transformation, a wide variation in copy number and insertion patterns exists (Jones et al, 1987; Jorgensen et al., 1987). Moreover, even within a single plant genotype, different patterns of T-DNA integration are possible based on the type of explant and transformation system used (Grevelding et al., 1993). Factors that regulate T-DNA copy number are poorly understood.

### B. Direct Gene Transfer

Direct plant transformation methods using DNA have also been reported. The first of these to be reported historically is electroporation, which utilizes an electrical current applied to a solution containing plant cells (M. E. Fromm et al., Nature, 319, 791 (1986); H. Jones et al., Plant Mol. Biol., 13, 501 (1989) and H. Yang et al., Plant Cell Reports, 7, 421 (1988).

Another direct method, called “biolistic bombardment”, uses ultrafine particles, usually tungsten or gold, that are coated with DNA and then sprayed onto the surface of a plant tissue with sufficient force to cause the particles to penetrate plant cells, including the thick cell wall, membrane and nuclear envelope, but without killing at least some of them (U.S. Pat. Nos. 5,204,253, 5,015,580).

A third direct method uses fibrous forms of metal or ceramic consisting of sharp, porous or hollow needle-like projections that literally impale the cells, and also the nuclear envelope of cells. Both silicon carbide and aluminum borate whiskers have been used for plant transformation (Mizuno et al., 2004; Petolino et al., 2000; U.S. Pat. No. 5,302,523 US Application 20040197909) and also for bacterial and animal transformation (Kaepler et al., 1992; Raloff, 1990; Wang, 1995). There are other methods reported, and undoubtedly, additional methods will be developed. The methods taught herein are capable of detecting the non-naturally occurring nucleotide junctions that result from any plant transformation method.

The T-DNA of the present disclosure may be transferred to any cell, for example, such as a plant cell transformation competent bacterium. Such bacteria are known in the art and may, for instance, belong to the following species: Agrobacterium spp., Rhizobium spp., Sinorhizobium spp., Mesorhizobium spp., Phyllobacterium spp. Ochrobactrum spp. and Bradyrhizobium spp. In some embodiments, such bacteria may belong to Agrobacterium spp. The T-DNA of the present disclosure may further comprise one or more replication origins for maintaining copies of the construct in E. coli. In some embodiments, origin of replication for maintaining copies of the construct in E. coli is derived from at least one of pBR322 and pUC.

The present disclosure also relates to a plant cell transforming bacterium comprising the T-DNA of the present disclosure, and which may be used for transforming a plant cell. In some embodiments, the plant transforming bacteria is selected from Agrobacterium spp., Rhizobium spp., Sinorhizobium spp., Mesorhizobium spp., Phyllobacterium spp. Ochrobactrum spp. or Bradyrhizobium spp.

The present disclosure also relates to a method for transforming a plant cell comprising: contacting at least a first plant cell with a plant cell transforming bacteria of the present disclosure; and selecting at least a plant cell transformed with the T-DNA. In some embodiments, the plant cell is a potato plant cell. In one embodiment, a method of the disclosure further comprises regenerating a plant from the plant cell.

The present disclosure also relates to a method of producing food, feed or an industrial product comprising: obtaining the plant of the present disclosure or a part thereof; and preparing the food, feed or industrial product from the plant or part thereof.

The disclosure also includes methods of genetically transforming plants with the T-DNA of the present disclosure and reducing the frequency of plants transformed with non-T DNA vector region. In some embodiments, the frequency of plants transformed with non-T-DNA region may be defined as less than or equal to about 20%. In some embodiments, the frequency is less than or equal to about 15%, in some embodiments less than about 10%, and in some embodiments less than or equal to about 8% or 5%. In some embodiments of the methods of the disclosure, the frequency of one- or two-copy T-DNA transformation events obtained is greater than or equal to about 70% or 75%. In some instances, that frequency can be raised to greater than or equal to about 80% or 85%, and in some embodiments, to greater than about 90% or 95%.

In some embodiments, the present disclosure provides a method for transforming a plant cell, wherein the method comprises: (i) introducing a plant transformation vector taught herein into the plant cell; and (ii) cultivating the transformed plant cell under conditions conducive to regeneration and mature plant growth. The plant cell is a potato plant cell.

The foregoing methods for transformation would typically be used for producing a transgenic variety. The transgenic variety could then be crossed with another (non-transformed or transformed) variety in order to produce a new transgenic variety. Alternatively, a genetic trait that has been engineered into a particular potato line using the foregoing transformation techniques could be moved into another line using traditional backcrossing techniques that are well known in the plant breeding arts. For example, a backcrossing approach could be used to move an engineered trait from a public, non-elite variety into an elite variety, or from a variety containing a foreign gene in its genome into a variety or varieties that do not contain that gene. As used herein, “crossing” can refer to a simple X by Y cross or the process of backcrossing depending on the context.

Persons of ordinary skill in the art will recognize that when the term potato plant is used in the context of the present disclosure, this also includes derivative varieties that retain the essential distinguishing characteristics of the event in question, such as a locus converted plant of that variety or a transgenic derivative having one or more value-added genes incorporated therein (such as herbicide or pest resistance).

Likewise, transgenes can be introduced into the plant using any of a variety of established recombinant methods well-known to persons skilled in the art, such as: Gressel, 1985, Biotechnologically Conferring Herbicide Resistance in Crops: The Present Realities, In Molecular Form and Function of the Plant Genome, L. van Vloten-Doting, (ed.), Plenum Press, New York; Huttner, S. L., et al., 1992, Revising Oversight of Genetically Modified Plants, Bio/Technology; Klee, H., et al., 1989, Plant Gene Vectors and Genetic Transformation: Plant Transformation Systems Based on the use of Agrobacterium tumefaciens, Cell Culture and Somatic Cell Genetics of Plants; Koncz, C., et al., 1986, The Promoter of T. sub.L-DNA Gene 5 Controls the Tissue-Specific Expression of Chimeric Genes Carried by a Novel Type of Agrobacterium Binary Vector; Molecular and General Genetics; Lawson, C., et al., 1990, Engineering Resistance to Mixed Virus Infection in a Commercial Potato Cultivar: Resistance to Potato Virus X and Potato Virus Y in Transgenic Russet Burbank, Bio/Technology; Mitsky, T. A., et al., 1996, Plants Resistant to Infection by PLRV. U.S. Pat. No. 5,510,253; Newell, C. A., et al., 1991, Agrobacterium-Mediated Transformation of Solanum tuberosum L. Cv. Russet Burbank, Plant Cell Reports; Perlak, F. J., et al., 1993, Genetically Improved Potatoes: Protection from Damage by Colorado Potato Beetles, Plant Molecular Biology; all of which are incorporated herein by reference for this purpose.

Many traits have been identified that are not regularly selected for in the development of a new variety but that can be improved by backcrossing and genetic engineering techniques. These traits may or may not be transgenic; examples of these traits include but are not limited to: herbicide resistance; resistance to bacterial, fungal or viral disease; insect resistance; uniformity or increase in concentration of starch and other carbohydrates; enhanced nutritional quality; decrease in tendency of tuber to bruise; and decrease in the rate of starch conversion to sugars. These genes are generally inherited through the nucleus.

### EXAMPLES

The foregoing examples of the related art and limitations related therewith are intended to be illustrative and not exclusive. Other limitations of the related art will become apparent to those of skill in the art upon a reading of the specification.

The present disclosure describes multi-cassette T-DNA and methods of integrating the described T-DNA into the genome of selected potato plant varieties to develop new intragenic potato plant varieties. The method includes trait identification, design of vectors, incorporation of vectors into Agrobacterium, selection of the recipient potato variety, plant transformation, and confirmation that the new potato plant varieties contain only the T-DNA.

### Example 1: pSIM4363 Transformation Vector Backbone

Plasmid pSIM4363 is a 38.9 kb binary transformation vector used to transform potatoes. This example shows the source of the genetic elements, the cloning steps for the backbone, and T-DNA sequences, and the order of the elements in the plasmid.

The various elements of the backbone are described in Table 1 and shown in FIG. 1.

TABLE 1
Genetic elements of the pSIM4363 Backbone

| Genetic | | Accession | | Size | |
|---|---|---|---|---|---|
| Element | Origin | Number¹ | Position | (bp) | Function |
| 1. Intervening | Synthetic DNA | | 29,957- | 6 | Sequence used for |
| sequence | | | 29,962 | | cloning |
| 2. Overdrive | Agrobacterium | NC_002377 | 29,963- | 30 | Enhances cleavage at |
| | tumefaciens Ti- | | 29,992 | | Right border site |
| | plasmid | | | | |
| 3. Intervening | Pseudomonas | AJ537514 | 29,993- | 1,082 | pVS1 backbone |
| sequence | fluorescens | | 31,074 | | |
| | pVS1 | | | | |
| 4. pVS1 | P. fluorescens | AJ537514 | 31,075- | 1,001 | pVS1 stability |
| partitioning | pVS1 | | 32,075 | | |
| protein StaA | | | | | |
| (PVS1 Sta) | | | | | |
| 5. Intervening | P. fluorescens | AJ537514 | 32,076- | 593 | pVS1 backbone |
| sequence | pVS1 | | 32,668 | | |
| 6. pVS1 | P. fluorescens | AJ537514 | 32,669- | 1,001 | pVS1 replication region |
| replicon | pVS1 | | 33,669 | | in Agrobacterium |
| (pVS1 Rep) | | | | | |
| 7. Intervening | P. fluorescens | AJ537514 | 33670- | 238 | pVS1 backbone |
| sequence | pVS1 | | 33,907 | | |
| 8. Intervening | pBR322 | AF234297 | 33,908- | 171 | pCambia1301 backbone |
| sequence | | | 34,078 | | |
| 9. pBR322 | pBR322 | AF234297 | 34,079- | 261 | pBR322 region for |
| bom | | | 34,339 | | replication in E. coli |
| 10. Intervening | pBR322 | AF234297 | 34,340- | 139 | pBR322 backbone |
| sequence | | | 34,478 | | |
| 11. Origin of | pBR322 | AF234297 | 34,479- | 281 | Region for replication in |
| replication | | | 34,759 | | E. coli |
| for pBR322 | | | | | |
| (pBR322 ori) | | | | | |
| 12. Intervening | pBR322 | AF234297 | 34,760- | 290 | pCambia1301 backbone |
| sequence | | | 35,049 | | |
| 13. Kanamycin | Staphylococcus | AF234297 | 35050- | 795 | Aminoglycoside |
| resistance | aureus | | 35,844 | | phosphotransferase-III |
| (KmR) gene | | | | | used as selection marker |
| | | | | | for cloning in bacteria. |
| 14. Intervening | Vector DNA | FJ362602 | 35,845- | 195 | pCambia vector |
| sequence | | | 36,039 | | backbone |
| 15. Promoter | Solanum | L22576 | 36,040- | 1,149 | Drives expression of ipt |
| of the | tuberosum | | 37,188 | | gene |
| ubiquitin-3 | | | | | |
| gene (pUbi3) | | | | | |
| 16. Intervening | Synthetic DNA | | 37,189- | 5 | Sequence used for |
| sequence | | | 37,193 | | cloning |
| 17. Isopentenyl | A. tumefaciens | NC_002377 | 37,194- | 723 | Screenable marker that |
| transferase | Ti-plasmid | | 37,916 | | catalyzes formation of |
| (ipt) gene | | | | | isopentenyl-AMP, a |
| | | | | | plant cytokinin causing |
| | | | | | abnormal plant growth |
| | | | | | phenotypes |
| 18. Intervening | A. tumefaciens | NC_002377 | 37,917- | 351 | Sequence used for DNA |
| sequence | Ti-plasmid | | 38,267 | | cloning |
| 19. Terminator | S. tuberosum | GP755544 | 38,268- | 355 | Terminator for ipt gene |
| of the | | | 38,622 | | transcription |
| ubiquitin-3 | | | | | |
| gene | | | | | |
| (tUbi3) | | | | | |
| 20. Intervening | Synthetic DNA | | 38,623- | 9 | Sequence used for DNA |
| sequence | | | 38,631 | | cloning |
| 21. Intervening | pBR322 | AF234297 | 38,632- | 244 | pCambia1301 backbone |
| sequence | | | 38,875 | | |
| 22. Intervening | Synthetic DNA | | 38,876- | 6 | Sequence used for DNA |
| sequence | | | 38,881 | | cloning |

The pSIM4363 backbone contains two well-characterized regions required for bacterial maintenance: pVS1 (Sta and Rep) for replication in Agrobacterium tumefaciens (Elements 3-7 in Table 1) and pBR322 (bom and ori) for replication in Escherichia coli (Elements 8-12 in Table 1).

Additional backbone elements include: Agrobacterium DNA overdrive sequence (Element 2 in Table 1) for more efficient cleavage at the Right border (RB) site (Toro et al., 1988), An aminoglycoside phosphotransferase III gene (Element 13 in Table 1) for use as a kanamycin selectable marker in bacteria (Gray and Fitch, 1983), and Agrobacterium isopentenyl transferase (ipt) gene (Element 17 in Table 1) for use as a screenable marker allowing negative selection of backbone containing events. Transcription is regulated by a Solanum tuberosum polyubiquitin 3 gene promoter and terminator (Elements 15 and 19 in Table 1) (Garbarino and Belknap, 1994).

The T-DNA Right border is efficiently cleaved by Agrobacterium, aided by the overdrive sequence that is part of the plasmid backbone. Cleavage at the Left border, however, is often less precise, where transfer of partial backbone sequences may occur along with the T-DNA. To screen out events that contain backbone sequence, the ipt expression cassette was included in the backbone.

When present in transformed plants, expression of ipt results in the overproduction of the plant hormone cytokinin causing plants to exhibit stunted phenotypes, abnormal leaf development, and an inability to root (Smigocki and Owens, 1988). Only plantlets phenotypically indistinguishable from untransformed controls are selected for further characterization. This screening reduces the possibility of transformed plants containing backbone DNA (Richael et al., 2008).

### Example 2: T-DNA of pSIM4363 Transformation Vector

The pSIM4363 T-DNA insert region including flanking Left and Right border sequences, is 29,956 bp long (from 1 bp to 29,956 bp). The pSIM4363 T-DNA insert is stably integrated into the potato genome. The pSIM4363 T-DNA insert or a functional part thereof, is the only genetic material of vector pSIM4363 that is integrated in the potato plant varieties of the disclosure, including, but not limited to Russet Norkotah TX 296 (NK) and Russet Burbank (RB).

The pSIM4363 T-DNA insert is illustrated and described in: FIG. 1 (along with vector backbone region), FIG. 2, and Table 2 below. The LB and RB sequences (25 bp each) were synthetically designed to be similar to and function like T-DNA borders from Agrobacterium tumefaciens.

Plasmid pSIM4363 T-DNA contains six cassettes: two silencing elements in the pSIM4363 T-DNA are designed as inverted repeats for the down regulation of gene expression through the RNA interference (RNAi) pathway. The first silencing/down-regulation cassette targets two potato proteins, polyphenol oxidase (PPO) for reduced black spot and vacuolar invertase (VINV) for lower reducing sugars (Elements 21-31 in Table 2). Transcription of the PPO/VInv inverted repeat is regulated by two inward facing promoters, the granule-bound starch synthase gene promoter (pGbss) and the ADP glucose pyrophosphorylase gene promoter (pAgp; Elements 21 and 31 in Table 2). The source of both promoter sequences is potato.

The second silencing element targets the gene encoding the coat protein of Potato Virus Y (PVY-Cp) and confers PVY protection to the plant (Elements 33-39 in Table 2). Transcription of the PVY-Cp inverted repeat is regulated by a polyubiquitin promoter (pUbi7; Element 33 in Table 2) and terminator (tUbi3; Element 39 in Table 2), both sourced from potato.

Three of the cassettes in pSIM4363 are designed to introduce broad spectrum and durable late blight protection through expression of three different potato resistance genes (R-genes): Rpi-vnt1 (Element 10 in Table 2) from S. venturii; Rpi-amr3 (Element 14 in Table 2) from S. americanum; and Rpi-blb2 (Element 18 in Table 2) from S. bulbocastanum.

Transcription of each R-gene is regulated by the native promoter and terminator sequences for that gene (e.g., Rpi-vnt1 is expressed using the native Rpi-vnt1 promoter and terminator sequences found in S. venturii): Rpi-vnt1 promoter and terminator (Elements 9 and 11 in Table 2) from S. venturii; Rpi-amr3 promoter and terminator (Elements 13 and 15 in Table 2) from S. americanum; and Rpi-blb2 promoter and terminator (Elements 17 and 19 in Table 2) from S. bulbocastanum.

The sixth cassette in the pSIM4363 T-DNA was designed to introduce a modified version of the potato acetolactate synthase gene (StmAls) into transformed plants for use as a selection marker during transformation. ALS is a key enzyme in the synthesis of branched-chain amino acids in all plant and microbial species. The protein (StmALS) produced from expression of the StmAls gene is two amino acids different from the wild-type ALS protein (StALS) (the StmALS and StALS protein sequences are 99% identical). The modified amino acids include tryptophan modified to leucine (W563L) and serine modified to isoleucine (S642I). Wild-type ALS is sensitive to herbicides that bind to the protein and block its activity, which causes the plant to die from the lack of essential branched-chain amino acids. The changes in amino acid sequence in StmALS make the protein insensitive to herbicides allowing selection of transformed events using herbicides.

TABLE 2
Genetic Elements of pSIM4363 T-DNA, from Left border Site to Right border

| | | Accession | | Size | Intended |
|---|---|---|---|---|---|
| Genetic Element | Origin | Number | Position | (bp) | Function |
| 1. Left border (LB) | | | | | |
| region | | | | | |
| A. LB site | Synthetic DNA | AY566555 | 1-25 | 25 | Secondary |
| | | | | | cleavage site |
| | | | | | releases |
| | | | | | ssDNA inserts |
| | | | | | from |
| | | | | | pSIM4363 |
| B. LB buffer | S. tuberosum | AY566555 | 26-187 | 162 | Buffer for |
| | var. Ranger | | | | truncations |
| | Russet. | | | | during |
| | | | | | insertion |
| 2. Intervening | S. tuberosum | AF393847 | 188-192 | 5 | Sequence used |
| sequence | | | | | for DNA |
| | | | | | cloning |
| 3. Polyubiquitin | S. tuberosum | U26831 | 193- | 1,736 | Drives |
| promoter (pUbi7) | var. Ranger | | 1,928 | | expression of |
| | Russet | | | | the StmAls |
| | | | | | gene |
| 4. Intervening | Synthetic DNA | | 1,929- | 6 | Sequence used |
| sequence | | | 1,934 | | for DNA |
| | | | | | cloning |
| 5. Modified Als gene | S. tuberosum | XM_006364617 | 1,935- | 1,980 | Selection |
| (StmAls) | | | 3,914 | | marker; |
| | | | | | modified |
| | | | | | potato ALS |
| | | | | | (StmALS) |
| 6. Intervening | Synthetic DNA | | 3,915- | 6 | Sequence used |
| sequence | | | 3,920 | | for DNA |
| | | | | | cloning |
| 7. Ubiquitin-3 gene | S. tuberosum | GP755544 | 3,921- | 355 | Terminates |
| terminator (tUbi3) | | | 4,275 | | transcription |
| | | | | | of StmAls gene |
| 8. Intervening | Synthetic DNA | | 4,276- | 10 | Sequence used |
| sequence | | | 4,285 | | for DNA |
| | | | | | cloning |
| 9. Rpi-vnt1 native | S. venturii | FJ423044 | 4,286- | 925 | Terminates |
| terminator (tVnt1) | | | 5,210 | | transcription |
| | | | | | of Rpi-vnt1 |
| | | | | | gene |
| 10. Rpi-vnt1 gene | S. venturii | FJ423044 | 5,211- | 2,676 | Expresses the |
| coding sequence | | | 7,886 | | VNT1 protein |
| (Vnt1) | | | | | for foliar late |
| | | | | | blight |
| | | | | | protection |
| 11. Rpi-vnt1 native | S. venturii | FJ423044 | 7,887- | 709 | Drives |
| promoter (pVnt1) | | | 8,595 | | expression of |
| | | | | | the Rpi-vnt1 |
| | | | | | gene |
| 12. Intervening | Synthetic DNA | | 8,596- | 6 | Sequence used |
| sequence | | | 8,601 | | for DNA |
| | | | | | cloning |
| 13. Rpi-amr3 native | S. americanum | KT373889.1 | 8,602- | 1,918 | Drives |
| promoter (pAmr3) | | | 10,519 | | expression of |
| | | | | | the Rpi-amr3 |
| | | | | | gene |
| 14. Rpi-amr3 gene | S. americanum | KT373889.1 | 10,520- | 2,664 | Expresses the |
| coding sequence | | | 13,183 | | AMR3 protein |
| (Amr3) | | | | | for late blight |
| | | | | | protection |
| 15. Rpi-amr3 native | S. americanum | KT373889.1 | 13,184- | 770 | Terminates |
| terminator (tAmr3) | | | 13,953 | | transcription |
| | | | | | of Rpi-amr3 |
| | | | | | gene |
| 16. Intervening | Synthetic DNA | | 13,954- | 12 | Sequence used |
| sequence | | | 13,965 | | for DNA |
| | | | | | cloning |
| 17. Rpi-blb2 native | S. | DQ122125 | 13,966- | 2,532 | Terminates |
| terminator (tBlb2) | bulbocastanum | | 16,497 | | transcription |
| | | | | | of Rpi-blb2 |
| | | | | | gene |
| 18. Rpi-blb2 gene | S. | DQ122125 | 16,498- | 3,890 | Expresses the |
| coding sequence | bulbocastanum | | 20,387 | | BLB2 protein |
| (Blb2) | | | | | for late blight |
| | | | | | protection |
| 19. Rpi-blb2 native | S. | DQ122125 | 20,388- | 1,545 | Drives |
| promoter (pBlb2) | bulbocastanum | | 21,932 | | expression of |
| | | | | | the Rpi-blb2 |
| | | | | | gene |
| 20. Intervening | Synthetic DNA | | 21,933- | 8 | Sequence used |
| sequence | | | 21,940 | | for DNA |
| | | | | | cloning |
| 21. Granule-bound | S. tuberosum | HM363755 | 21,941- | 686 | Drives |
| starch synthase gene | var. Ranger | | 22,626 | | expression of |
| promoter (pGbss) | Russet | | | | the VInv/Ppo |
| | | | | | inverted repeat |
| 22. Intervening | Synthetic DNA | | 22,627- | 7 | Sequence used |
| sequence | | | 22,633 | | for DNA |
| | | | | | cloning |
| 23. Vacuolar | S. tuberosum | DQ478950 | 22,634- | 504 | Generates |
| invertase gene | var. Ranger | | 23,137 | | dsRNA to |
| fragment (sense | Russet | | | | down regulate |
| orientation; VInv) | | | | | invertas |
| | | | | | transcripts |
| | | | | | (VInv) |
| 24. Intervening | Synthetic DNA | | 23,138- | 6 | Sequence used |
| sequence | | | 23,143 | | for DNA |
| | | | | | cloning |
| 25. 3′-untranslated | S. verrucosum | HM363754 | 23,144- | 144 | Forms dsRNA |
| region of the | | | 23,287 | | to reduce |
| polyphenol oxidase | | | | | expression of |
| gene (antisense | | | | | polyphenol |
| orientation; Ppo) | | | | | oxidase to |
| | | | | | reduce black |
| | | | | | spot |
| 26. Spacer 1 | S. tuberosum | HM363753 | 23,288- | 163 | Sequence |
| | var. Ranger | | 23,450 | | between the |
| | Russet | | | | inverted |
| | | | | | repeats; forms |
| | | | | | loop in |
| | | | | | dsRNA |
| | | | | | transcript |
| 27. 3′-untranslated | S. verrucosum | HM363754 | 23,451- | 144 | Forms dsRNA |
| region of the | | | 23,594 | | to reduce |
| polyphenol oxidase | | | | | expression of |
| gene (sense | | | | | polyphenol |
| orientation; Ppo) | | | | | oxidase to |
| | | | | | reduce black |
| | | | | | spot |
| 28. Intervening | Synthetic DNA | | 23,595- | 7 | Sequence used |
| sequence | | | 23,601 | | for DNA |
| | | | | | cloning |
| 29. Vacuolar | S. tuberosum | DQ478950 | 23,602- | 498 | Generates |
| invertase gene | var. Ranger | | 24,099 | | dsRNA to |
| fragment (antisense | Russet | | | | down regulate |
| orientation, VInv) | | | | | invertase |
| | | | | | transcripts |
| | | | | | (VInv) |
| 30. Intervening | Synthetic DNA | | 24,100- | 6 | Sequence used |
| sequence | | | 24,105 | | for DNA |
| | | | | | cloning |
| 31. ADP glucose | S. tuberosum | HM363752 | 24,106- | 2,260 | Drives |
| pyrophosphorylase | var. Ranger | | 26,365 | | expression of |
| gene promoter | Russet | | | | VInv/Ppo |
| (pAgp) | | | | | inverted repeat |
| 32. Intervening | Synthetic DNA | | 26,366- | 12 | Sequence used |
| sequence | | | 26,377 | | for DNA |
| | | | | | cloning |
| 33. Polyubiquitin | S. tuberosum | U26831 | 26,378- | 1,736 | Drives |
| promoter (pUbi7) | var. Ranger | | 28,113 | | expression of |
| | Russet | | | | the PVY-Cp |
| | | | | | inverted repeat |
| 34. Intervening | Synthetic DNA | | 28,114- | 6 | Sequence used |
| sequence | | | 28,119 | | for DNA |
| | | | | | cloning |
| 35. Potato virus Y | Potato virus Y | AJ890342 | 28,120- | 522 | Generates |
| coat protein gene | strain N | | 28,641 | | dsRNA that |
| fragment (sense | | | | | target PVY |
| orientation, PVY- | | | | | genome for |
| Cp) | | | | | degradation |
| 36. Spacer 2 | S. tuberosum | HM363755 | 28,642- | 237 | Gbss intron |
| | var. Ranger | | 28,878 | | sequence |
| | Russet | | | | between PVY- |
| | | | | | Cp inverted |
| | | | | | repeat to form |
| | | | | | loop in |
| | | | | | dsRNA |
| 37. Potato virus Y | Potato virus Y | AJ890342 | 28,879- | 522 | Generates |
| coat protein gene | strain N | | 29,400 | | dsRNA that |
| fragment (antisense | | | | | target PVY |
| orientation, PVY- | | | | | genome for |
| Cp) | | | | | degradation |
| 38. Intervening | Synthetic DNA | | 29,401- | 6 | Sequence used |
| sequence | | | 29,406 | | for DNA |
| | | | | | cloning |
| 39. Ubiquitin-3 gene | S. tuberosum | GP755544 | 29,407- | 355 | Terminator for |
| terminator (tUbi3) | | | 29,761 | | PVY-Cp |
| | | | | | dsRNA |
| | | | | | transcript |
| 40. Intervening | Synthetic DNA | | 29,762- | 9 | Sequence used |
| sequence | | | 29,770 | | for DNA |
| | | | | | cloning |
| 41. Right border | | | | | |
| (RB) region | | | | | |
| A. RB buffer | S. tuberosum | AY566555 | 29,771- | 161 | Supports |
| | var. Ranger | | 29,931 | | primary |
| | Russet | | | | cleavage at RB |
| B. RB site | Synthetic | AY566555 | 29,932- | 25 | Primary |
| | | | 29,956 | | cleavage site |
| | | | | | releases |
| | | | | | ssDNA insert |
| | | | | | from |
| | | | | | PSIM4363 |

Thus, as can be seen from Table 1 and Table 2, the pSIM4363 plasmid is a binary vector designed for potato plant transformation. The vector backbone contains sequences for replication in both E. coli and Agrobacterium along with an ipt marker for screening to eliminate plants with vector backbone DNA. The T-DNA region consists of two expression cassettes flanked by LB and RB sequences. Upon inoculation of host plant tissue with Agrobacterium containing the pSIM4363 vector, the T-DNA region of pSIM4363 is transferred into the host genome.

The pSIM4363 plasmid was assembled using standard molecular biology techniques. Sequences used in the assembly of the cassettes in pSIM4363 are shown in Table 1 and Table 2 as intervening sequences. These intervening sequences include restriction enzyme recognition sites used for cutting and ligation of separate sequence fragments into a single transformation plasmid.

The pSIM4363 plasmid is a binary vector designed for potato plant transformation. The vector backbone includes elements for replication in both E. coli and A. tumefaciens, along with the ipt gene used as a screenable marker for identifying plants containing backbone DNA. The T-DNA consists of six cassettes designed to express inverted repeats or gene coding sequences. These sequences are designed to provide late blight protection, Potato virus Y protection, reduced sugars, and reduced black spot. The entire T-DNA is flanked by Left border and Right border regions necessary for Agrobacterium-mediated insertion into the potato genome.

### Example 3: T-DNA of pSIM4617 Transformation Vector

In this example, a new cloning system was utilized to insert DNA cassettes into the engineered disarmed virulence plasmid of Agrobacterium to generate pSIM4617. This system is known as Gene Assembly in Agrobacterium by Nucleic acid Transfer using Recombinase TechnologY (GAANTRY) and is described in Collier, R. et al (2018), Plant Journal 95, 573-583.

In the field of plant transformation, Agrobacterium contain a virulence plasmid that produces a T-DNA that is transferred into a plant cell and causes transformation. In Agrobacterium tumefaciens, this is the Ti plasmid. In Agrobacterium rhizogenes this is the Ri plasmid. Previously it was difficult to generate engineered virulence plasmids because of the large size and the need to complete the molecular engineering in the Agrobacterium. Further complicating molecular engineering, virulence plasmids cannot be propagated in E. coli. As a consequence, binary vectors were developed that are smaller than the virulence plasmids, contain elements that allow replication in E. coli, and contain T-DNA left and right borders that flank the desired cassettes to be delivered to the plant cell upon transformation. The binary vectors are engineered and maintained in E. coli, and then introduced into Agrobacterium for plant transformation, which provides for transfer of the binary vector T-DNA into the plant cell.

The GAANTRY cloning procedure for pSIM4617 construction is outlined in the below. The final pSIM4617 T-DNA stack is StmALS+VntI+Amr3+Blb2+PPO+INV+PVY, as detailed in Table 3 and FIG. 3.

TABLE 3
Genetic Elements of pSIM4617 T-DNA, from Left border Site to Right border

| | | | Accession | Position | Size | Intended |
|---|---|---|---|---|---|---|
| | Genetic Element | Origin | Number | (pSIM4617) | (bp) | Function |
| 1 | Left Border site | Synthetic DNA | AY566555 | 1-25 | 25 | Secondary |
| | (LB) | | | | | cleavage site |
| | | | | | | releases |
| | | | | | | ssDNA |
| | | | | | | inserts from |
| | | | | | | pSIM4617 |
| 2 | Intervening | Recombination | | 26-203 | 177 | GAANTRY |
| | Sequence | of GAANTRY | | | | recombination |
| | | attB/attP | | | | footprints |
| | | donors | | | | used for DNA |
| | | | | | | cloning |
| 3 | Polyubiquitin | S. tuberosum | U26831 | 204-1,941 | 1738 | Drives |
| | promoter B | var. Ranger | | | | expression of |
| | (pUbi7) | Russet | | | | the StmALS |
| | | | | | | gene |
| 4 | Intervening | Synthetic DNA | | 1,942- | 6 | Sequence |
| | Sequence | | | 1,947 | | used for DNA |
| | | | | | | cloning |
| 5 | Modified StALS | S. tuberosum | XM_006364617 | 1948-3927 | 1980 | Used as a |
| | gene (StmALS) | | | | | selection |
| | | | | | | marker |
| 6 | Intervening | Synthetic DNA | | 3,928-3,933 | 6 | Sequence |
| | Sequence | | | | | used for DNA |
| | | | | | | cloning |
| 7 | Terminator of the | S. tuberosum | GP755544 | 3,934-4,288 | 355 | Terminates |
| | ubiquitin-3 gene | | | | | the |
| | (tUbi3) | | | | | transcription |
| | | | | | | of StmALS |
| | | | | | | gene |
| | | | | | | (Garbarino |
| | | | | | | and Belknap, |
| | | | | | | 1994) |
| 8 | Intervening | pSIM5102 | | 4,289-4,340 | 52 | Sequence |
| | Sequence | | | | | used for DNA |
| | | | | | | cloning |
| 9 | Rpi-vnt1 gene | S. venturii | FJ423044 | 4,341-5,049 | 709 | Drives |
| | promoter (pVnt1) | | | | | expression of |
| | | | | | | the Rpi-vnt1 |
| | | | | | | gene |
| 10 | Rpi-vnt1 gene | S. venturii | FJ423044 | 5,050-7,725 | 2676 | Expresses the |
| | coding sequence | | | | | protein of |
| | (Vnt1) | | | | | Rpi-vnt1 gene |
| 11 | Rpi-vnt1 gene | S. venturii | FJ423044 | 7,726-8,650 | 925 | Terminates |
| | terminator (tVnt1) | | | | | transcription |
| | | | | | | of Rpi-vnt1 |
| | | | | | | gene |
| 12 | Intervening | Recombination | | 8651-8996 | 345 | Recombined |
| | Sequence | of GAANTRY | | | | foot prints |
| | | attB/attP | | | | used for DNA |
| | | donors | | | | cloning |
| 13 | Rpi-amr3 gene | S. americanum | KT373889.1 | 8996-10913 | 1918 | Drives |
| | promoter (pAmr3) | | | | | expression of |
| | | | | | | the Rpi-amr3 |
| | | | | | | gene |
| 14 | Rpi-amr3 gene | S. americanum | KT373889.1 | 10914- | 2664 | Expresses the |
| | coding sequence | | | 13577 | | protein of |
| | (Amr3) | | | | | amr3 gene |
| 15 | Rpi-amr3 gene | S. americanum | KT373889.1 | 13578- | 770 | Terminates |
| | terminator (tAmr3) | | | 14347 | | transcription |
| | | | | | | of Rpi-amr3 |
| | | | | | | gene |
| 16 | Intervening | Synthetic DNA | | 14348- | 12 | Sequence |
| | Sequence | | | 14359 | | used for DNA |
| | | | | | | cloning |
| 17 | Rpi-blb2 gene | S. | DQ122125 | 14360- | 2,532 | Terminates |
| | terminator (tBlb2) | bulbocastanum | | 16891 | | transcription |
| | | | | | | of Rpi-blb2 |
| | | | | | | gene |
| 18 | Rpi-blb2 gene | S. | DQ122125 | 16892- | 3,890 | Expresses the |
| | coding sequence | bulbocastanum | | 20781 | | protein of |
| | (Blb2) | | | | | Rpi-blb2 gene |
| 19 | Rpi-blb2 gene | S. | DQ122125 | 20782- | 1,545 | Drives |
| | promoter (pBlb2) | bulbocastanum | | 22326 | | expression of |
| | | | | | | the Rpi-blb2 |
| | | | | | | gene |
| 20 | Intervening | Recombination | | 22327- | 342 | GAANTRY |
| | Sequence | of GAANTRY | | 22668 | | recombination |
| | | attB/attP | | | | footprints |
| | | donors | | | | used for DNA |
| | | | | | | cloning |
| 21 | Granule-bound | S. tuberosum | HM363755 | 22669- | 686 | Drives |
| | starch synthase | var. Ranger | | 23354 | | expression of |
| | gene promoter | Russet | | | | an inverted |
| | (pGbss) | | | | | repeat |
| | | | | | | fragments of |
| | | | | | | VInv, |
| | | | | | | especially in |
| | | | | | | tubers |
| 22 | Intervening | Synthetic DNA | | 23355- | 7 | Sequence |
| | Sequence | | | 23361 | | used for DNA |
| | | | | | | cloning |
| 23 | VInv gene | S. tuberosum | DQ478950 | 23362- | 504 | Generates a |
| | fragment (sense | var. Ranger | | 23865 | | dsRNA to |
| | orientation; VInv) | Russet | | | | down regulate |
| | | | | | | VInv |
| | | | | | | transcripts |
| 24 | Intervening | Synthetic DNA | | 23866- | 6 | Sequence |
| | Sequence | | | 23871 | | used for DNA |
| | | | | | | cloning |
| 25 | VInv gene | S. tuberosum | DQ478950 | 23872- | 679 | Generates a |
| | fragment | var. Ranger | | 24550 | | dsRNA to |
| | (antisense | Russet | | | | down regulate |
| | orientation; VInv) | | | | | VInv |
| | | | | | | transcripts |
| 26 | Intervening | Synthetic DNA | | 24551- | 6 | Sequence |
| | Sequence | | | 24556 | | used for DNA |
| | | | | | | cloning |
| 27 | ADP glucose | S. tuberosum | HM363752 | 24557- | 2260 | Drives |
| | pyrophosphorylase | var. Ranger | | 26816 | | expression of |
| | gene promoter | Russet | | | | an inverted |
| | (pAgp) | | | | | repeat |
| | | | | | | fragments of |
| | | | | | | VInv, |
| | | | | | | especially in |
| | | | | | | tubers |
| 28 | Intervening | Synthetic DNA | | 26817- | 6 | Sequence |
| | Sequence | | | 26822 | | used for DNA |
| | | | | | | cloning |
| 29 | Granule-bound | S. tuberosum | HM363755 | 26823- | 403 | Drives |
| | starch synthase | var. Ranger | | 27225 | | expression of |
| | gene promoter | Russet | | | | an inverted |
| | without leader | | | | | repeat |
| | (pGbssNL) | | | | | fragments of |
| | | | | | | Ppo, |
| | | | | | | especially in |
| | | | | | | tubers |
| 30 | Intervening | Synthetic DNA | | 27226- | 12 | Sequence |
| | Sequence | | | 27237 | | used for DNA |
| | | | | | | cloning |
| 31 | 3’-untranslated | S. verrucosum | HM363754 | 27238- | 144 | Forms |
| | sequence of the | | | 27381 | | dsRNA to |
| | polyphenol | | | | | reduce |
| | oxidase-gene (in | | | | | expression of |
| | antisense | | | | | polyphenol |
| | orientation; Ppo) | | | | | oxidase to |
| | | | | | | reduce black |
| | | | | | | spot |
| 32 | Polyphenol oxidase | S. tuberosum | | 27382- | 215 | Loop |
| | coding sequence | var. Russet | | 27596 | | sequence of |
| | | Burbank | | | | the ppo gene |
| | | | | | | inverted |
| | | | | | | repeat |
| 33 | Intervening | Synthetic DNA | | 27597- | 7 | Sequence |
| | Sequence | | | 27603 | | used for DNA |
| | | | | | | cloning |
| 34 | 3’-untranslated | S. verrucosum | HM363754 | 27604- | 144 | Forms |
| | sequence of the | | | 27747 | | dsRNA to |
| | polyphenol oxidase | | | | | reduce |
| | gene (in sense | | | | | expression of |
| | orientation; Ppo) | | | | | polyphenol |
| | | | | | | oxidase to |
| | | | | | | reduce black |
| | | | | | | spot |
| 35 | Intervening | Synthetic DNA | | 27748- | 12 | Sequence |
| | Sequence | | | 27759 | | used for DNA |
| | | | | | | cloning |
| 36 | ADP glucose | S. tuberosum | HM363752 | 27760- | 2260 | Drives |
| | pyrophosphorylase | var. Ranger | | 30019 | | expression of |
| | gene promoter | Russet | | | | the Ppo |
| | (pAgp) | | | | | inverted |
| | | | | | | repeats, |
| | | | | | | especially in |
| | | | | | | tubers |
| 37 | Intervening | pSIM5105 | | 30020- | 40 | Sequence |
| | Sequence | | | 30059 | | used for DNA |
| | | | | | | cloning |
| 38 | Polyubiquitin | S. tuberosum | U26831 | 30060- | 1434 | Drives |
| | promoter with RR | var. Ranger | | 31493 | | expression of |
| | intron (pUbi7int) | Russet | | | | the PVY-Cp |
| | | | | | | gene |
| 39 | Intervening | Synthetic DNA | | 31494- | 9 | Sequence |
| | Sequence | | | 31502 | | used for DNA |
| | | | | | | cloning |
| 40 | Potato virus Y | Potato virus Y | AJ890342 | 31503- | 522 | Generates a |
| | strain coat protein | strain N | | 32024 | | dsRNA to |
| | gene fragment | | | | | silence PVY- |
| | (sense orientation; | | | | | Cp gene |
| | PVY-Cp) | | | | | |
| 41 | Intron of Solanum | S. tuberosum | HM363755 | 32025- | 237 | Non-coding |
| | tuberosum granule | var. Ranger | | 32261 | | sequence |
| | bound starch | Russet | | | | between |
| | synthase (Gbss) | | | | | PVY-Cp |
| | gene | | | | | inverted |
| | | | | | | repeats to |
| | | | | | | form loop in |
| | | | | | | dsRNA |
| 42 | Potato virus Y | Potato virus Y | AJ890342 | 32262- | 522 | Generates a |
| | strain coat protein | strain N | | 32783 | | dsRNA to |
| | gene fragment | | | | | silence PVY- |
| | (antisense | | | | | Cp gene |
| | orientation; PVY- | | | | | |
| | Cp) | | | | | |
| 43 | Intervening | Synthetic DNA | | 32784- | 6 | Sequence |
| | Sequence | | | 32789 | | used for DNA |
| | | | | | | cloning |
| 44 | Terminator of the | S. tuberosum | GP755544 | 32790- | 355 | Terminator |
| | ubiquitin-3 gene | | | 33144 | | for PVY-Cp |
| | (tUbi3) | | | | | dsRNA |
| | | | | | | transcript |
| 45 | Intervening | Recombination | | 33145- | 133 | GAANTRY |
| | Sequence | of GAANTRY | | 33277 | | recombination |
| | | attB/attP | | | | footprints |
| | | donors | | | | used for DNA |
| | | | | | | cloning |
| 46 | Right border (RB) | Synthetic DNA | AY566555 | 33278- | 43 | Primary |
| | site | | | 33302 | | cleavage site |
| | | | | | | releases |
| | | | | | | ssDNA insert |
| | | | | | | from |
| | | | | | | pSIM4617 |
| | | | | | | (VanHaaren |
| | | | | | | etal., 1989) |

### Example 4: Transformation of Agrobacterium Strain with pSIM4363

The C58-derived Agrobacterium strain AGL1 was developed by precisely deleting the transfer DNA of the hyper-virulent plasmid pTiBo542 (Lazo et al., 1991). A transposon insertion in the general recombination gene (recA) stabilizes recombinant plasmid vectors such as pSIM4363 (FIG. 1). AGL1 displays resistance against carbenicillin and rifampicin, and is eliminated from transformed potato tissue using timentin. Following selection, plants are both antibiotic and Agrobacterium free, with the potato-derived expression cassettes inserted into the plant's genome.

Stock plants were maintained in magenta boxes with 40 ml half-strength M516 (Phytotechnology) medium containing 3% sucrose and 2 g/l gelrite (propagation medium). Potato internode segments of four to six mm were cut from four-week old plants, infected with the Agrobacterium AGL1 strain carrying pSIM4363, and transferred to tissue culture media containing 3% sucrose and 6 g/l agar (co-cultivation medium). Infected explants were transferred, after two days, to M404 (Phytotechnology) medium containing 3% sucrose, 6 g/l agar and 150 mg/l timentin to eliminate Agrobacterium (hormone-free medium). Details of the methods are described in Richael et al. (2008).

After one month, the infected explants were transferred to fresh medium lacking any synthetic hormones and incubated in a Percival growth chamber under a 16 hr photoperiod at 24° C. where they started to form shoots. Many shoots expressed the ipt gene and displayed a cytokinin overproduction phenotype; these shoots were not considered for further analyses. PCR genotyping demonstrated that about 0.3 to 1.5% of the remaining shoots contained at least part of the T-DNA while lacking the ipt gene. Thus, no markers were used to select for the transformed plants. Details on ipt-based marker-free plant transformation were published by Richael et al. (2008).

The process of eliminating Agrobacterium started two days after explant infection. For this purpose, tissues were subjected to the antibiotic timentin (150 mg/L) until proven to be free of live Agrobacterium. Proof was obtained by incubating stem fragments of transformed events on nutrient broth-yeast extract (NBY medium) for 2 weeks at 28° C. (repeated twice). In accordance with 97 CFR Part 340, transformed plants were transported and planted in the field only when free of live Agrobacterium.

Potato varieties Russet Norkotah TX 296 (NK) and Russet Burbank (RB) were transformed with pSIM4363 giving rise to events NA-16 and event BG-115, respectively.

### Example 5: Plant Transformation Detection (pSIM4363)

An event detection method was developed using polymerase chain reaction (PCR) to provide detection of pSIM4363 in samples of potato leaf of Potato varieties Russet Norkotah TX 296 (NK) and Russet Burbank (RB).

All DNA was isolated using a cetyltrimethyl ammonium bromide (CTAB) method as follows:

### DNA Isolation Method

(1) For leaf samples: Grind 1-2 g of tissue to fine powder in a small size mortar using liquid nitrogen. (2) Transfer the powder to a pre-cooled (−80° C. freezer or liquid nitrogen) 15 mL conical tube with a pre cooled (in liquid nitrogen) spatula. Store in −80° C. freezer until ready to process. (3) Add 10 mL of extraction buffer (350 mM sorbitol, 100 mM Tris, 50 mM EDTA, pH 8) and mix until tissue powder is thoroughly thawed. (4) Centrifuge at 3,000 rpm for 15 min at room temperature. (5) Pour off the supernatant. (Rinse the pellet with 2 mL extraction buffer if necessary). (6) Add 2 mL extraction buffer and 2 μL 100 mg/mL RNase A to the pellet. Resuspend the pellet thoroughly by vortexing the tube. (7) Add 2 mL of nuclear lysis buffer (200 mM Tris, 50 mM EDTA, 2 M NaCl, pH 7.5-8, 2% CTAB) and 800 μL 5% sarcosyl. (8) Mix by inverting ten times. (9) Incubate the mixture at 65° C. for 20 min. Mix again after the first 10 min. (10) Add 6 mL (or equal volume) of chloroform:isoamyl alcohol (24:1). (11) Mix by shaking for about 1 min. (12) Centrifuge at 3,000 rpm for 5 min at room temperature. (13) Transfer aqueous phase into a 15 mL conical tube. (14) Repeat steps 10-12 at least one time until the aqueous phase is clear. (15) Precipitate DNA with 5 mL (or equal volume) of isopropyl alcohol. (16) Gently invert tubes until DNA comes out of solution. (17) Centrifuge at 3,000 rpm for 10 min at room temperature. (18) Discard the supernatant. (19) Rinse the pellet with 70% ethanol (room temperature). (20) Centrifuge at 3,000 rpm for 5 min at room temperature. (21) Pour off supernatant and carefully transfer pellet to 1.5 mL microcentrifuge tube. (22) Centrifuge at 14,800 rpm for 1 min. Remove excess liquid and leave caps open to let pellet air dry. (23) Resuspend the DNA pellet in 400 leaf μL TE buffer (pH 8.0). (24) Measure DNA concentration using a fluorescent intercalating dye (e.g., Qubit dsDNA BR Assay Kit from Life Technologies) and samples can b e tested for PCR inhibitors through serial dilution analysis.

### PCR Reaction Conditions

TABLE 4
PCR Reaction Setup and Conditions (40 μL total volume)

| Reagent | Final Concentration | 1X Sample (μL)¹ |
|---|---|---|
| AccuStart II PCR | 1X | 20 |
| Supermix (Quanta) | | |
| Forward primer (10 μM) | 0.4 μM | 1.5 |
| Reverse primer (10 μM) | 0.4 μM | 1.5 |
| Template DNA (15 ng/μL) | 22.5 ng | 1.5 |
| H₂O | — | 15.5 |

¹5 μL of the PCR reaction was loaded on a 3% agarose gel for anaysis.

TABLE 5
PCR Thermal Cycling Conditions

| Cycles | Temperature (° C.) | Time (s) |
|---|---|---|
| 1 | 95 | 300 |
| 32 | 95 | 30 |
| | 60 | 30 |
| | 68 | 30 |
| 1 | 68 | 600 |

### Primer and Probe Development

Primers were designed to amplify a region that is specific to a junction between cassettes within the pSIM4363 T-DNA, specifically between the Amr3 terminator and the Blb2 terminator sequences (FIG. 2, Table 6). Primers specific to Adenine Phosphoribosyl Transferase (APRT) from Solanum tuberosum were used as an endogenous, positive control (Table 6). APRT was chosen based on its common use as a potato endogenous control in qPCR studies (Nicot, Hausman, Hoffmann, & Evers, 2005).

TABLE 6
Assay Specific Oligonucleotide
Sequences for PCR Analysis

| | | | Oligo | |
|---|---|---|---|---|
| | | SEQ ID | nucleotide | Amplicon |
| Assay | Primer | No. | Sequence | Size (bp) |
| pSIM4363 | SPS- | SEQ ID | 5′-CGTTCCTCT | 220 |
| | pSIM4363- | NO: 4 | CAACTTAATC-3′ | |
| | F | | | |
| | SPS- | SEQ ID | 5′-GCTTAAATT | |
| | pSIM4363- | NO: 5 | CTAGATCCCC-3′ | |
| | R | | | |
| APRT | SPS-APRT- | SEQ ID | 5′-GAACCGGAGC | 121 |
| | F | NO: 6 | AGGTGAAGAA-3′ | |
| | SPS-APRT- | SEQ ID | 5′-GAAGCAATCC | |
| | R | NO: 7 | CAGCGATACG-3′ | |

Specificity of pSIM4363 Primers and Probe

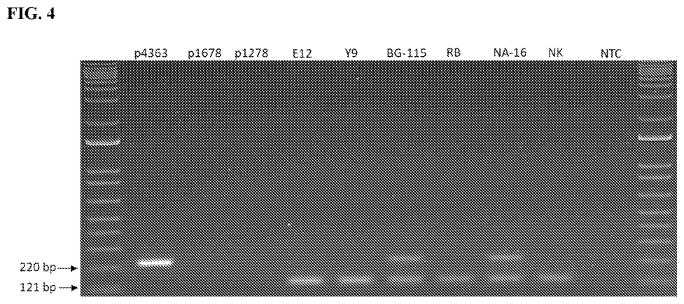

Potato varieties Russet Norkotah TX 296 (NK) and Russet Burbank (RB) have been transformed with pSIM4363 giving rise to event NA-16 and event BG-115, respectively. The assay developed to detect pSIM4363 transformed events is specific and does not recognize other Simplot events transformed with pSIM1278 and pSIM1678, which contain different T-DNA. Transformation with pSIM1278 in RB has produced event E12 (U.S. Pat. No. 9,328,352), and transformation with pSIM1278 and pSIM1678 in Atlantic has produced event Y9 (U.S. Pat. No. 9,968,043). To demonstrate the specificity of the pSIM4363 assay, PCR was conducted on plasmid DNA from pSIM1278, pSIM1678, and pSIM4363, genomic DNA from events E12, Y9, NA-16, and BG-115, as well as genomic DNA from NK and RB conventional varieties, using the pSIM4363-specific primer set (FIG. 4, Table 6). The endogenous potato gene APRT assay (Table 6) was utilized as an amplification positive control. The pSIM4363 assay produces a 220 bp fragment while the APRT assay produces a 121 bp fragment, resolved by agarose gel electrophoresis (FIG. 4).

The detection methods in this example show that events transformed with pSIM4363 can be detected or identified in potato leaves using PCR. This assay was shown to be specific for the pSIM4363-transformed events in potato leaf DNA when challenged using pSIM1278- and pSIM1678-transformed events and conventional varieties.

### Example 6: Construction of pSIM4617 Transformation Vector and Preparation of Electro-Competent Cell

The following protocol is for preparation of electro-competent Agrobacterium at each step of the GAANTRY stacking procedure.

(1) Inoculate 2-3 ml of LB with containing appropriate antibiotics and incubate for about 16-24 h at 28-30° C. with shaking at 250 rpm. (2) Next, inoculate 25 ml of LB containing appropriate antibiotics in a 125 ml flask with 250 μl culture and incubate for about 15 h at 28-30° C. (3) Divide the culture into two 50 ml tubes and cool on ice. (4) Centrifuge both tubes of cells at 4000×g for 10 min at 4° C. to pellet the cells. (5) Remove as much as possible of supernatant from the cell pellets. (6) Gently suspend each of the cell pellets in 25 ml of ice-cold filter-sterilized Milli-Q water. (7) From here throughout the procedure, maintain the tubes on ice. (8) Pellet the cells again by centrifugation at 4000×g for 10 min at 4° C. (9) Decant and re-suspend each of the cell pellets in 12.5 ml of ice-cold water. (Keeping the tubes on ice.) (10) Pellet the cells again by centrifugation at 4000×g for 10 min at 4° C. (11) Decant and re-suspend each of the pellets in 6.3 ml of ice-cold 10% glycerol (Mix water and 100% glycerol at 9:1 v:v) and combine the two cell suspensions in a single tube. (Keeping the tubes on ice.) (12) Pellet the cells again by centrifugation at 4000×g for 10 min at 4° C. (13) Remove as much as possible of the supernatant from the cell pellet. (14) Re-suspend the final cell pellet in 200 μl of ice-cold 10% glycerol, while keeping the tubes on ice. (15) Dispense the cells in 20 μl aliquots in pre-chilled, sterile 1.5 ml tubes on ice using a pre-chilled pipet tip. Freeze in liquid nitrogen. Store at −80° C. (16) These prepared cells are good for approximately 6 months.

Electroporation Protocol for the Stacking of Genes into the GAANTRY Vector

(1) Pre-chill a 1 mm gap electroporation cuvette on ice. (2) Set the MicroPluser (Bio-Rad, Hercules, Calif., USA) to “Agr” (2.2 kV, 10 μF, and 600Ω). (3) Prepare 250 μl of SOC (Super Optimal broth with Catabolite repression, Invitrogen, Waltham, Mass., USA) in a sterile 1.5 ml tube at room temperature (RT). (4) Thaw a 20 μl aliquot tube of competent cells on ice. (5) Chill DNA on ice if it is prepared just before electroporation. (6) Add DNA up to 4 μl and tap briefly. (7) Transfer the mix of cells and DNA to the pre-chilled cuvette. (8) Tap on bench top to remove air bubbles. (9) Place the cuvette in the chamber slide, push place with contacts. (10) Pulse once. (11) Immediately add 200 μl of SOC to cuvette, suspend cells, and quickly transfer cells the tube containing 50 μl of SOC. (12) Incubate the tube at 28-30° C. for 4 h with intermittent gentle shaking. (13) Plate 100 μl of cells onto the appropriate selective media plates (selection based on the particular step of the stack) Incubate at 28-30° C. for 2-3 days.

### GAANTRY Gene Stacking

The GAANTRY cloning system was used to build pSIM4617 in a three-step stacking protocol largely as described in Collier (2018).

### Stack 1

The first round inserted the two genes of interest (GOI) StmAls and Vnt1 in one cassette (rows 2-12 of Table 3). For this stacking step, the StmAls and Vnt1 cassette was cloned into the multiple cloning site (MCS) of a B-DONOR plasmid (pB-DONOR-1). As described in Collier (2018), a B-HELPER plasmid (pB-HELPER) was used for the stacking step.

A 20 μl aliquot of electro-competent cells was thawed on ice, and DNA (up to 5 μl) of pB-DONOR-1 and pB-HELPER at a ratio of 20 ng B-Donor-1:20 ng pB-HELPER was mixed. The cell-DNA aliquot was transferred to a pre-chilled 1 mm-gap cuvette. Electroporation was done as described, after which 250 μl of SOC was added. This was incubated 28-30° C. for 30-60 min., and then 100 μl was spread onto an LB+200 mg/L Gentamycin plate, which was incubated at 28-30° C. for 2-3 days.

Next, for the sucrose screening step, four well-isolated single colonies were picked and touched lightly with a tip of toothpick. This was streaked in a quarter (¼) area of LB+5% sucrose+Rifamycin (Rif) 25+100 mg/L Gentamycin. A clean toothpick was used to do secondary streaking from the first streak. The streaked plates were incubated at 28-30° C. for 2-3 days.

For counter selection, two well-isolated single colonies from each line from the sucrose plates. The cells from the isolated colonies were streaked on counter-selective medium, which for a pB-Donor was LB+100 mg/L Kanamycin. Plates were incubated at 28-30° C. for 2-3 days. These plates should remain free of any growth.

Isolated colonies from the same colony chosen for counter selection (that showed no growth) were inoculated into LB+Gentamycin and incubated at 28-30° C. for 24 h. DNA was isolated from the culture using the Gram Negative Protocol of Gentra Puregene Yeast/Bacterial Kit (QIAGEN, Germantown, Md., USA). A portion of the culture was retained for glycerol stock to prepare cells for stack 2. To confirm successful stack 1, PCR analysis was done on the isolated DNA with the primer sets in Table 7. The PCR reaction for checking the stack is: DNA 2 μl, 2× Accustart II 5 μl (AccuStart II PCR Supermix, QuantaBio, Beverly, Mass., USA), Forward Primer 0.375 μl, Reverse Primer 0.375 μl, Water 4.25 μl, for a total reaction volume of 12 μl. The PCR cycling parameters were: Initial denature 95° C.-3 min; Denature 95° C.-20 sec; Anneal 56° C.-40 sec; Extension 68° C.-2 min; 39 cycles; hold 10° C. PCR reaction products run on agarose gels. Agrobacterium with all correct PCR products were selected for successful stack 1 steps and used for stack 2.

TABLE 7
Stack 1 PCR check

| PCR Reaction No. | SEQ ID NOS | | Stack region | PCR product size |
|---|---|---|---|---|
| 1 | 8 | 9 | Ubi7-ALS | 279 bp |
| 2 | 10 | 11 | ALS-Ubi3T | 1331 bp |
| 3 | 12 | 13 | Vnt1 | 1564 bp |
| 4 | 14 | 15 | Vnt-Pvnt1 | 1179 bp |

### Stack 2

The second round of GAANTRY stacking was used to insert the Amr3 and Blb2 cassettes (rows 12-20 of Table 3). For this stacking step, the Amr3 and Blb2 cassettes were cloned into the multiple cloning site (MCS) of a P-DONOR plasmid (P-DONOR-1). As described in Collier (2018), a P-HELPER plasmid (pP-HELPER) was used for the stacking step.

Electro-competent Agrobacterium containing stack 1 were prepared as per the protocol detailed above. A 20 μl aliquot of the stack 1 electro-competent cells was thawed on ice, and DNA (up to 5 μl) of P-DONOR-1 and pP-HELPER at a ratio of 20 ng P-Donor-1:60 ng pP-HELPER was mixed. The cell-DNA aliquot was transferred to a pre-chilled 1 mm-gap cuvette. Electroporation was done as described, after which 250 μl of SOC was added. This was incubated 28-30° C. for 30-60 min., and then 100 μl was spread onto an LB+200 mg/L Kanamycin plate, which was incubated at 28-30° C. for 2-3 days.

Next, for the sucrose screening step, well-isolated single colonies were picked and touched lightly with a tip of toothpick. This was streaked in a quarter (¼) area of LB+5% sucrose+Rifamycin (Rif) 25+100 mg/L Kanamycin. A clean toothpick was used to do secondary streaking from the first streak. The streaked plates were incubated at 28-30° C. for 2-3 days.

For counter selection, two well-isolated single colonies from each line from the sucrose plates were selected. The cells from the isolated colonies were streaked on counter-selective medium, which for a pB-Donor was LB+100 mg/L Gentamycin. Plates were incubated at 28-30° C. for 2-3 days. These plates should remain free of any growth.

Isolated colonies from the same colony chosen for counter selection (that showed no growth) were inoculated into LB+Kanamycin and incubated at 28-30° C. for 24 h. DNA was isolated from the culture using the Gram Negative Protocol of Gentra Puregene Yeast/Bacterial Kit (QIAGEN, Germantown, Md., USA). A portion of the culture was retained for glycerol stock to prepare cells for stack 3. To confirm successful stack 2, PCR analysis was done on the isolated DNA with the primer sets in Table 8. The PCR reaction for checking the stack is: DNA 2 μl, 2× Accustart II 5 μl, Forward Primer 0.375 μl, Reverse Primer 0.375 μl, Water 4.25 μl, for a total reaction volume of 12 μl. The PCR cycling parameters were: Initial denature 95° C.-3 min; Denature 95° C.-20 sec; Anneal 56° C.-40 sec; Extension 68° C.-2 min; 39 cycles; hold 10° C. PCR reaction products run on agarose gels. Agrobacterium with all correct PCR products were selected for successful stack 1 and stack 2 steps and used for stack 3.

TABLE 8
Stack 2 PCR check

| PCR Reaction No. | SEQ ID NOS | | Stack region | PCR product size |
|---|---|---|---|---|
| 1 | 8 | 9 | Ubi7-ALS | 279 bp |
| 2 | 10 | 11 | ALS-Ubi3T | 1331 bp |
| 3 | 12 | 13 | Vnt1 | 1564 bp |
| 4 | 14 | 15 | Vnt-Pvnt1 | 1179 bp |
| 5 | 16 | 17 | Amr3 | 1385 bp |
| 6 | 18 | 19 | Amr3-Tamr3 | 1432 bp |
| 7 | 20 | 21 | Tblb2-blb2 | 1224 bp |
| 8 | 22 | 23 | Blb2-Pblb2 | 1154 bp |
| 9 | 24 | 25 | Blb2 middle | 1627 bp |
| 10 | 26 | 27 | Blb2-GBSS | 588 bp |

### Stack 3

The third round of GAANTRY stacking was used to insert the remaining cassettes of pSIM4617 (rows 20-45 of Table 3). For this stacking step, the expression cassettes for silencing/downregulating VInv, GBSS, Ppo and the expression cassette for PVY-Cp were cloned into the multiple cloning site (MCS) of a B-DONOR plasmid (pB-DONOR-2). As described in Collier (2018), a B-HELPER plasmid (pB-HELPER) was used for this stacking step.

A 20 μl aliquot of electro-competent cells was thawed on ice, and DNA (up to 5 μl) of pB-DONOR-2 and pB-HELPER at a ratio of 20 ng B-Donor-2:20 ng pB-HELPER was mixed. The cell-DNA aliquot was transferred to a pre-chilled 1 mm-gap cuvette. Electroporation was done as described, after which 250 μl of SOC was added. This was incubated 28-30° C. for 30-60 min., and then 100 μl was spread onto an LB+200 mg/L Gentamycin plate, which was incubated at 28-30° C. for 2-3 days.

Next, for the sucrose screening step, four well-isolated single colonies were picked and touched lightly with a tip of toothpick. This was streaked in a quarter (¼) area of LB+5% sucrose+Rifamycin (Rif) 25+100 mg/L Gentamycin. A clean toothpick was used to do secondary streaking from the first streak. The streaked plates were incubated at 28-30° C. for 2-3 days.

For counter selection, two well-isolated single colonies from each line from the sucrose plates. The cells from the isolated colonies were streaked on counter-selective medium, which for a pB-Donor was LB+100 mg/L Kanamycin. Plates were incubated at 28-30° C. for 2-3 days. These plates should remain free of any growth.

Isolated colonies from the same colony chosen for counter selection (that showed no growth) were inoculated into LB+Gentamycin and incubated at 28-30° C. for 24 h. DNA was isolated from the culture using the Gram Negative Protocol of Gentra Puregene Yeast/Bacterial Kit (QIAGEN, Germantown, Md., USA). A portion of the culture was retained for glycerol stock. To confirm successful stack 3, PCR analysis was done on the isolated DNA with the primer sets in Table 9. The PCR reaction for checking the stack is: DNA 2 2× Accustart II 5 μl Forward Primer 0.375 μl, Reverse Primer 0.375 μl, Water 4.25 μl, for a total reaction volume of 12 μl. The PCR cycling parameters were: Initial denature 95° C.-3 min; Denature 95° C.-20 sec; Anneal 56° C.-40 sec; Extension 68° C.-2 min; 39 cycles; hold 10° C. PCR reaction products run on agarose gels. Glycerol stocks of the single colony with all correct PCR products of Table 9 are pSIM4617 containing Agrobacterium were made. This Agrobacterium comprising the pSIM4617 T-DNA was used for plant tissue transformation.

TABLE 9
Stack 3 PCR check

| PCR Reaction No. | SEQ ID NOS | | Stack region | PCR product size |
|---|---|---|---|---|
| 1 | 8 | 9 | Ubi7-ALS | 279 bp |
| 2 | 10 | 11 | ALS-Ubi3T | 1331 bp |
| 3 | 12 | 13 | Vnt1 | 1564 bp |
| 4 | 14 | 15 | Vnt-Pvnt1 | 1179 bp |
| 5 | 16 | 17 | Amr3 | 1385 bp |
| 6 | 18 | 19 | Amr3-Tamr3 | 1432 bp |
| 7 | 20 | 21 | Tblb2-blb2 | 1224 bp |
| 8 | 22 | 23 | Blb2-Pblb2 | 1154 bp |
| 9 | 24 | 25 | Blb2 middle | 1627 bp |
| 10 | 26 | 27 | Blb2-GBSS | 588 bp |
| 11 | 28 | 29 | INV/AGP | 434 bp |
| 12 | 30 | 31 | Ppo/AGP | 354 bp |
| 13 | 32 | 33 | AGP/Ubi7 | 600 bp |
| 14 | 34 | 35 | Cp-Ubi3T | 654 bp |

P-Donor and B-Donor constructs containing the desired DNA cassettes are prepared in the correct donor backbone so that the stacking proceeds in the predicted predetermined order in the final T-DNA. Alternatively, each DNA cassette can be placed in both P-donor and B-donor vectors to have the flexibility to use the DNA cassette at any step in the stacking process. Upon completion of each round of a stacking step, the construction is tested by PCR for the insertion of the DNA cassette to confirm that the stack was successful, instead of the typical enzymatic digestion to simply check by band size due to the immense size of the virulence plasmid. With each round, after the stack is confirmed, competent cells are made for the subsequent steps and continued stacking of DNA cassettes.

### Example 7: Plant Transformation Detection (pSIM4617)

Potato explants were transformed with pSIM4617 containing Agrobacterium per the usual transformation protocol. After selection on ALS containing medium, tissue samples were taken from plants with rootlets. The DNA was extracted from this tissue sample and screened by qPCR for the presence or absence of each GOI of pSIM4617. The qPCR cycling parameters are: Initial denature 95 C-3 min; Denature 95 C-15 sec; Anneal 56 C-20 sec; Extension 68 C-30 sec; 39 cycles; end. The PCR primers and probes for this screen are listed in Table 10. The Ref-VPS is the reference/control probe for the assay.

TABLE 10
qPCR primers and probes for screening transformed plantlets

| T-DNA Target | SEQ ID NO | Primer/Probe |
|---|---|---|
| Ubi7P-ALS | 36 | Forward Primer |
| Ubi7P-ALS | 37 | Reverse Primer |
| Ubi7P-ALS | 38 | Probe |
| Ubi3T-VntI | 39 | Forward Primer |
| Ubi3T-VntI | 40 | Reverse Primer |
| Ubi3T-VntI | 41 | Probe |
| amr3T-blb2T | 42 | Forward Primer |
| amr3T-blb2T | 43 | Reverse Primer |
| amr3T-blb2T | 44 | Probe |
| blb2-Footprint | 45 | Forward Primer |
| blb2-Footprint | 46 | Reverse Primer |
| blb2-Footprint | 47 | Probe |
| AGP-Inv | 48 | Forward Primer |
| AGP-Inv | 49 | Reverse Primer |
| AGP-Inv | 50 | Probe |
| PPO2-AGP | 51 | Forward Primer |
| PPO2-AGP | 52 | Reverse Primer |
| PPO2-AGP | 53 | Probe |
| PVY-Ubi3T | 54 | Forward Primer |
| PVY-Ubi3T | 55 | Reverse Primer |
| PVY-Ubi3T | 56 | Probe |
| Ref-VPS | 57 | Forward Primer |
| Ref-VPS | 58 | Reverse Primer |
| Ref-VPS | 59 | Probe |
| Left border (LB) | 60 | Forward Primer |
| LB | 61 | Reverse Primer |
| LB | 62 | Probe |
| Right border (RB) | 63 | Forward Primer |
| RB | 64 | Reverse Primer |
| RB | 65 | Probe |

Plants with rootlets that were identified with the qPCR screening to contain each of the pSIM4617 T-DNA regions by qPCR were selected for further evaluation. After cultivation in a greenhouse, the plants were evaluated for copy number and locus within the plant genome of the pSIM4617 T-DNA. For this, plant tissue was collected, DNA extracted, PCR ran, and the droplet digital PCR reader (ddPCR, BioRad) was used to determine copy number and locus. The PCR cycling parameters for this analysis was: Initial denature 95 C-3 min; Denature 95 C-15 sec; Anneal 56 C-20 sec; Extension 68 C-30 sec; 44 cycles; hold at 4 C. The PCR primers and probes for this screen are listed in Table 11. Plants selected with a single copy of each GOI in the pSIM4617 T-DNA were selected for further evaluation.

TABLE 11
ddPCR primers and probes for
screening transformed plants for copy number

| T-DNA Target | SEQ ID NO | Primer/Probe |
|---|---|---|
| Ubi7P-ALS | 66 | Forward Primer |
| Ubi7P-ALS | 67 | Reverse Primer |
| Ubi7P-ALS | 68 | Probe |
| Ubi3T-VntI | 69 | Forward Primer |
| Ubi3T-VntI | 70 | Reverse Primer |
| Ubi3T-VntI | 71 | Probe |
| FP-Amr3T | 72 | Forward Primer |
| FP-Amr3T | 73 | Reverse Primer |
| FP-Amr3T | 74 | Probe |
| amr3T-blb2T | 75 | Forward Primer |
| amr3T-blb2T | 76 | Reverse Primer |
| amr3T-blb2T | 77 | Probe |
| blb2-FP | 78 | Forward Primer |
| blb2-FP | 79 | Reverse Primer |
| blb2-FP | 80 | Probe |
| FP-GBSS | 81 | Forward Primer |
| FP-GBSS | 82 | Reverse Primer |
| FP-GBSS | 83 | Probe |
| AGP-Inv | 84 | Forward Primer |
| AGP-Inv | 85 | Reverse Primer |
| AGP-Inv | 86 | Probe |
| PPO2-AGP | 87 | Forward Primer |
| PPO2-AGP | 88 | Reverse Primer |
| PPO2-AGP | 89 | Probe |
| GBSS-PPO | 90 | Forward Primer |
| GBSS-PPO | 91 | Reverse Primer |
| GBSS-PPO | 92 | Probe |
| Ubi7PRR-PVY | 93 | Forward Primer |
| Ubi7PRR-PVY | 94 | Reverse Primer |
| Ubi7PRR-PVY | 95 | Probe |
| PVY-Ubi3T | 96 | Forward Primer |
| PVY-Ubi3T | 97 | Reverse Primer |
| PVY-Ubi3T | 98 | Probe |
| Ref-VI | 99 | Forward Primer |
| Ref-VI | 100 | Reverse Primer |
| Ref-VI | 101 | Probe |

### Example 8: Screening Transformants and Analyzing Expression/Repression of Target Genes in Selected Transformants (pSIM4363 or pSIM4617)

1002481 Agrobacterium-based transformation of a potato variety with either pSIM4363 or pSIM4617 would entail the creation of at least 300 events that are known to have the complete T-DNA insert. Typically, this would require transforming about 32,000 stem explants. From these explants would be collected approximately 2400 shoots. Primary transformants are screened for the presence of the T-DNA insert and the absence of backbone DNA using a combination of PCR-based methods. At this stage about 290 independent, PCR positive events would be characterized for biochemical trait efficacy and disease resistance as explained in Examples 9-10. Events not meeting a particular quality standard for trait efficacy are eliminated from further characterization. Events satisfying criteria for trait efficacy are later screened by affinity capture next generation sequencing to characterize the locus of insertion, the copy number of the insert and insert complexity. qPCR or Northern blots follow to verify expression of appropriate transcript(s) associated with each expression cassette (e.g. Rpi-vnt1, Rpi-amr3, and Rpi-Blb2). Also, expression cassettes designed to express inverted repeats (sPPO and sVINV) for repressing/silencing endogenous potato genes are assessed using qPCR or biochemical assays to show the efficacy of those cassettes. Further, the inverted repeat containing PVY sequence is assessed by investigating efficacy against PVY infections in pathology studies.

After full characterization of events using molecular, biochemical and pathological tests, about 290 events will have been reduced to approximately 30 events that then go to field studies to assess field performance. From the 30 lines entering field trials at several locations, it is expected that a few (e.g. one or two) events will provide total tuber yields comparable to the untransformed control.

### Example 9: Field Performance and Tuber Evaluation

In Year 1, field trials were conducted with eight Russet Norkotah lines transformed with the pSIM4363 construct (NA lines). The best performing NA lines were identified based on field performance at five locations where Russet Norkotah potatoes are normally grown in the United States.

No differences among lines were observed for emergence rate at 30 days after planting using the FY1 seed source. Emergence was complete by 45 days after planting in Year 1 and no differences among the lines were detected. All lines reached greater than 95% emergence (Table 12).

TABLE 12
Year 1 Emergence Across Sites at Approximately 30 Days After Planting

| | Emergence (%) | |
|---|---|---|
| Line | 30 days after planting | 45 days after planting |
| R. Norkotah | 41.5 | 98.9 |
| NA16 | 40.8 | 98.8 |
| NA157 | 38.1 | 98.3 |
| NA68 | 35.0 | 98.3 |
| NA41 | 34.8 | 98.1 |
| NA4 | 32.7 | 98.0 |
| NA150 | 28.1 | 97.8 |
| NA40 | 20.2 | 97.8 |
| NA109 | 16.0 | 97.3 |

Total yield at the Year 1 sites is shown in Table 13. The top performing transformed lines were NA68, NA157, NA150, NA41, NA40, and NA16. These lines had yields similar to the Russet Norkotah untransformed control. As shown in Table 13, all selected NA lines were not different from each other except for NA109.

TABLE 13
Year 1 Yield Across Sites

| Line | Yield (cwt/a) |
|---|---|
| R. Norkotah control | 700.3 |
| NA68 | 676.4 |
| NA157 | 660.1 |
| NA150 | 630.8 |
| NA41 | 629.8 |
| NA40 | 628.2 |
| NA16 | 605.2 |
| NA4 | 593.2* |
| NA109 | 502.5* |

*Represents a statistically significant (p < 0.05) difference in yield compared to the control.

The strongest performing transformed lines were NA68, NA157, and NA40 when compared to the untransformed control. NA41, NA16, NA150, and NA4 yielded less than the control but were not different than the other NA lines except NA109.

In Year 1, the top four NA lines and the Russet Norkotah untransformed control were graded from two sites (Table 14). The tubers from IDIDAH were overall smaller than those from WAEPHR. This was likely due to a colder spring at the IDIDAH site. No significant differences were detected among lines within any tuber size category at the IDIDAH site. In WAEPHR, no differences were noted in the 4-6, 6-10, or 10-14 oz categories. In the <4 oz category, NA157 and NA68 had significantly more small tubers compared to the other lines. This trend was also observed in the >14 oz category.

TABLE 14
Year 1 Grading Results from the IDIDAH and WAEPHR Sites

| Site | Tuber Size | Line | Percent |
|---|---|---|---|
| IDIDAH | <4 oz | NA157 | 29.8 |
| | | NA150 | 27.8 |
| | | NA68 | 26.5 |
| | | NA41 | 24.4 |
| | | R. NORKOTAH | 20.7 |
| | 4-6 oz | NA150 | 28.8 |
| | | NA68 | 28.6 |
| | | NA157 | 27.0 |
| | | NA41 | 25.9 |
| | | R. NORKOTAH | 24.8 |
| | 6-10 oz | R. NORKOTAH | 38.8 |
| | | NA41 | 33.9 |
| | | NA68 | 33.1 |
| | | NA150 | 31.1 |
| | | NA157 | 29.8 |
| | 10-14 oz | NA41 | 13.1 |
| | | R. NORKOTAH | 12.3 |
| | | NA68 | 10.5 |
| | | NA157 | 10.5 |
| | | NA150 | 9.4 |
| | >14 oz | R. NORKOTAH | 3.4 |
| | | NA150 | 3.0 |
| | | NA157 | 3.0 |
| | | NA41 | 2.8 |
| | | NA68 | 1.3 |
| WAEPHR | <4 oz | NA157 | 11.4* |
| | | NA68 | 8.2 |
| | | NA41 | 5.8 |
| | | R. NORKOTAH | 5.5 |
| | | NA150 | 4.2 |
| | 4-6 oz | NA157 | 15.5* |
| | | NA68 | 10.8 |
| | | R. NORKOTAH | 9.8 |
| | | NA150 | 9.4 |
| | | NA41 | 8.6 |
| | 6-10 oz | NA157 | 32.2 |
| | | NA68 | 28.9 |
| | | R. NORKOTAH | 27.7 |
| | | NA41 | 23.4 |
| | | NA150 | 18.4 |
| | 10-14 oz | NA157 | 25.5 |
| | | NA41 | 24.9 |
| | | NA68 | 24.5 |
| | | NA150 | 23.0 |
| | | R. NORKOTAH | 22.5 |
| | >14 oz | NA150 | 45.0 |
| | | NA41 | 37.3 |
| | | R. NORKOTAH | 34.5 |
| | | NA68 | 27.6 |
| | | NA157 | 15.5* |

*Represents a statistically significant (p < 0.05) difference in tuber size compared to the control.

Specific gravity was determined for the same samples that were graded (Table 15). No differences were detected among the lines for Year 1 at the WAEPHR site in terms of the specific gravity values, which were consistent with the previous year results.

Higher specific gravities were also observed for Year 1 at the IDIDAH site. This is likely due to the smaller, less mature tubers harvested there. Nevertheless, all NA lines were not different than the Russet Norkotah control.

TABLE 15
Year 1 Specific Gravity from the IDIDAH and WAEPHR Sites

| Site | Line | Specific Gravity |
|---|---|---|
| 19-IDIDAH | NA150 | 1.090 |
| 19-IDIDAH | NA157 | 1.088 |
| 19-IDIDAH | R. NORKOTAH | 1.086 |
| 19-IDIDAH | NA41 | 1.085 |
| 19-IDIDAH | NA68 | 1.084 |
| 19-WAEPHR | R. NORKOTAH | 1.069 |
| 19-WAEPHR | NA41 | 1.067 |
| 19-WAEPHR | NA150 | 1.066 |
| 19-WAEPHR | NA157 | 1.065 |
| 19-WAEPHR | NA68 | 1.065 |

Samples from the Year 1 IDIDAH site were used in the controlled bruise test (Table 16). A controlled bruise evaluation was conducted using a 100 tuber sample of tubers harvested from field plots. A custom built jig designed to drop a weight on each tuber applied 0.74 newton-meters of energy to the apical and basal end of each tuber. Bruising was evaluated 24 hours afterward and the number of bruises, their color, and size were recorded. Significant differences were found for the number of bruises that developed on lines 41, 68 and 157. The bruises that developed on all of the NA lines were significantly smaller in size and less dark in color than the untransformed control line (R. NORKOTAH), which provides evidence that the reduction in Polyphenol oxidase in the selected transformants (i.e. NA lines in Table 16) is effective at minimizing the incidence and severity of black spot bruising.

TABLE 16
2019 Controlled Bruise Results from the IDIDAH Site

| | Number of Bruises | Size of Bruises, % | | | Color of Bruises, % | | |
|---|---|---|---|---|---|---|---|
| Line | on 100 Tubers | Small | Medium | Large | White | Brown | Black |
| NA150 | 46 | 95.6 | 4.4 | 0.0 | 17.2 | 82.8 | 0.0 |
| NA157 | 40* | 83.0 | 17.0 | 0.0 | 16.0 | 84.1 | 0.0 |
| NA41 | 31* | 76.8 | 23.2 | 0.0 | 18.3 | 81.7 | 0.0 |
| NA68 | 44* | 88.1 | 12.0 | 0.0 | 19.4 | 80.6 | 0.0 |
| NK296 | 64 | 64.7 | 35.3 | 0.0 | 1.1 | 83.4 | 15.4 |

*Represents a statistically significant (p < 0.05) difference in bruising compared to the control.

### Example 10: Biochemical and Pathological Characteristics of Potato Cultivar(s)/Plant(s) Transformed with pSIM4363 and/or pSIM4617

Russet Norkotah lines transformed with pSIM4363 (NA lines) were evaluated for late blight and potato virus Y (PVY) resistance in controlled laboratory tests. The parent variety, Russet Norkotah as the untransformed control is known to be susceptible to both pathogens. Indigenous strains of both Phytophthora infestans and PVY were collected from fields across the US to ensure NA line performance against relevant strains.

### (i) PVY Resistance Scores

Selected NA lines were evaluated against three strains of PVY common to the US, which are PVY^{N-Wi}, PVY^{O}, and PVY^{NTN}. Inoculum was rubbed onto lower leaves of plantlets with an abrasive powder to facilitate epidermal wounding and permit entry of the virus. Six weeks after inoculation virus titer was quantified using TAS-ELISA. While Russet Norkotah controls were susceptible, no virus was detected in several NA lines that harbor the T-DNA insert of pSIM4363 (Table 17).

TABLE 17
Reaction of NA lines to 3 strains of PVY in lab tests.

| Genotype | PVY^{NTN} | PVY^{O} | PVY^{N-Wi} |
|---|---|---|---|
| | − | − | − |
| NA150 | − | − | − |
| NA157 | − | − | − |
| NA41 | − | − | − |
| NA68 | − | − | − |
| R. NORKOTAH | + | + | + |

### (ii) Late Blight Resistance Scores

Selected NA lines were evaluated against seven P. infestans isolates differing in virulence and ability to identify expression and function of the 3 Rpi genes used. For example, strain EC1 is not recognized by Rpi-vnt1. Any line with functional Rpi-blb2 or Rpi-amr3 activity is expected to be resistant against P. infestans isolates.

Spray inoculation with sporangia concentrations of 5×10⁵ were used on young plantlets having 6-7 true leaves. Ten days after infection, lines tested in this example were scored by an objective scaling 1 to 7; 1 being susceptible and 7 being resistant. Resistance values are represented as an average of 3 replicates per line in Table 18 with all NA lines exhibiting a high degree of resistance to all strains of P. infestans. All lines had a degree of defoliation at such high concentrations of inoculum, but only older leaves were affected.

TABLE 18
Reaction of NA lines to 3 strains of PVY in lab tests. Numerals
are average reaction of 3 replicates per line.

| | Reaction against P. infestans isolates | | | | |
|---|---|---|---|---|---|
| Genotype | NL08797 | EC1 | NL13015 | NL07434 | NL14277 |
| NA150 | 5.7 | 7.0 | 5.0 | 6.3 | 5.0 |
| NA157 | 6.0 | 6.3 | 5.0 | 5.7 | 5.0 |
| NA41 | 5.7 | 7.0 | 5.0 | 5.7 | 7.0 |
| NA68 | 5.7 | 6.3 | 5.0 | 5.7 | 7.0 |
| R. NORKOTAH | 1.0 | 1.5 | 1.1 | 1.1 | 1.2 |

Tubers of Russet Norkotah lines transformed with pSIM4363 (NA lines) were evaluated for biochemical trait efficacy. Both polyphenol oxidase and vacuolar invertase activity were expected to be reduced in NA lines because of RNA inference methods employed in pSIM4363 to repress or silence expression of the relevant enzymes, and eventually suppress their activity.

Polyphenol oxidase (PPO) activity is high in the Russet Norkotah variety and accounts for an active browning reaction in tubers of the variety that are cut or peeled. The variety is also prone to blackspot bruising at sites of physical trauma during harvest and post-harvest handling. PPO is down-regulated in the pSIM4363 transformed Russet Norkotah lines. A benchtop spectrometer method was be used to score NA tuber extracts for PPO activity. Results verified a significant reduction in PPO activity in lines known/verified to have pSIM4363 T-DNA. While measure of control tubers indicated 123 units of PPO activity, NA lines ranged from 4 to 6.5 units of activity, which is a significant and meaningful reduction as seen in both slower browning reactions in peeled tubers and lower blackspot bruising in field-grown NA lines.

Invertase enzymes, including vacuolar invertase (VInv), hydrolyze sucrose into glucose and fructose. Glucose and fructose can accumulate in tubers during cold storage in a process known as cold-induced sweetening. If potatoes contain high levels of reducing sugars, they can become undesirably brown and develop bitter flavors after frying. Proof-of-concept experiments demonstrated that the most pronounced effect on reducing sugar levels could be accomplished by down regulation of VInv. High throughput methods enabled the rapid quantification of sucrose and reducing sugars in tuber samples from NA lines harboring pSIM4363 inserts engineered with a silencing element for VInv.

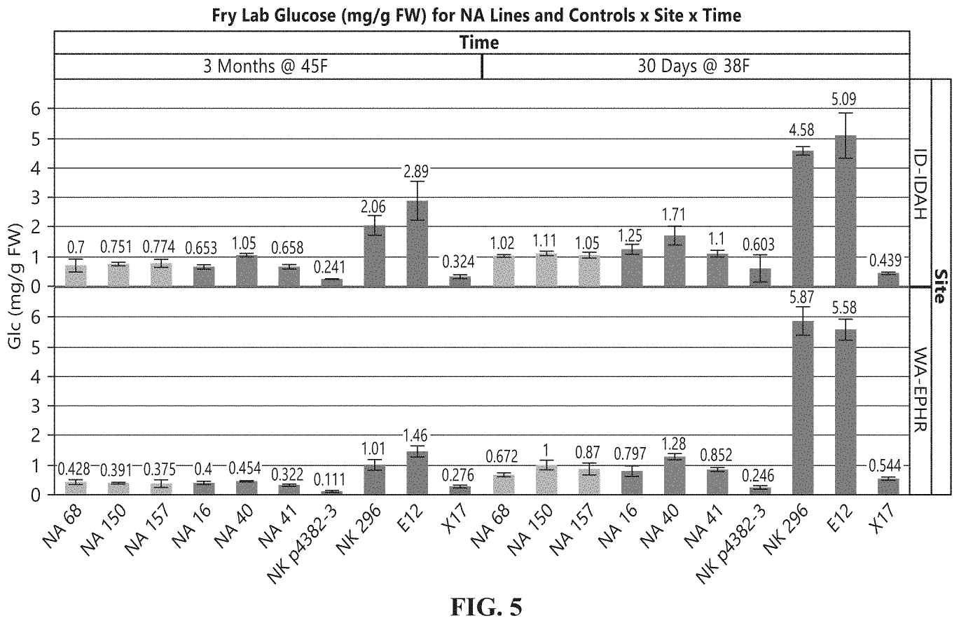

FIG. 5 illustrates that glucose accumulated to a high concentration in Russet Norkotah tubers when tubers are stored under two different cold conditions. When tubers were stored at 45° F. for 3 months, glucose concentrations rise slowly in NA lines known to have down-regulated VInv activity. Under more extreme cold, glucose levels in most NA lines are ¼ (i.e. 25%) of the control. A reduction in glucose levels correlates to more French fries being light colored. Fewer French fries are discarded for dark fry color.

### DEPOSIT INFORMATION

Vector deposits of the J. R. Simplot Company proprietary pSIM4363 and pSIM4617 disclosed above and recited in the appended claims has been made with the American Type Culture Collection (ATCC), 10801 University Boulevard, Manassas, Va. 20110. The date of deposit was made on Mar. 25, 2020. The deposit of vectors was taken from the same deposit maintained by J.R. Simplot Company since prior to the filing date of this application. All restrictions will be irrevocably removed upon granting of a patent, and the deposit is intended to meet all of the requirements of 37 C.F.R. §§ 1.801-1.809. The ATCC Accession Numbers for pSIM4363 and pSIM4617 are PTA-126737 and PTA-126738, respectively. The deposit will be maintained in the depository for a period of thirty years, or five years after the last request, or for the enforceable life of the patent, whichever is longer, and will be replaced as necessary during that period.

Tuber deposits of the J.R. Simplot Company proprietary Potato Cultivars/Events disclosed herewith will be made with an international depositary authority under the Budapest Treaty. The deposit will be taken from the same deposit maintained by J.R. Simplot Company. All restrictions will be irrevocably removed upon granting of a patent, and the deposit is intended to meet all of the requirements of 37 C.F.R. §§ 1.801-1.809. The deposit will be maintained in the depository for a period of thirty years, or five years after the last request, or for the enforceable life of the patent, whichever is longer, and will be replaced as necessary during that period.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

It should be understood that the above description is only representative of illustrative embodiments and examples. For the convenience of the reader, the above description has focused on a limited number of representative examples of all possible embodiments, examples that teach the principles of the disclosure. The description has not attempted to exhaustively enumerate all possible variations or even combinations of those variations described. That alternate embodiments may not have been presented for a specific portion of the disclosure, or that further undescribed alternate embodiments may be available for a portion, is not to be considered a disclaimer of those alternate embodiments. One of ordinary skill will appreciate that many of those undescribed embodiments, involve differences in technology and materials rather than differences in the application of the principles of the disclosure. Accordingly, the disclosure is not intended to be limited to less than the scope set forth in the following claims and equivalents.

### REFERENCES

Garbarino, J. E., and Belknap, W. R. (1994). Isolation of a Ubiquitin-Ribosomal Protein Gene (Ubi3) from Potato and Expression of Its Promoter in Transgenic Plants. Plant Molecular Biology 24, 119-127.
Richael, C. M., Kalyaeva, M., Chretien, R. C., Yan, H., Adimulam, S., Stivison, A., Weeks, J. T., and Rommens, C. M. (2008). Cytokinin Vectors Mediate Marker-Free and Backbone-Free Plant Transformation. Transgenic Research 17, 905-917.
Smigocki, A. C., and Owens, L. D. (1988). Cytokinin Gene Fused with a Strong Promoter Enhances Shoot Organogenesis and Zeatin Levels in Transformed Plant Cells. PNAS 85, 5131-5135.
Toro, N., Datta, A., Yanofsky, M., and Nester, E. (1988). Role of the Overdrive Sequence in T-DNA Border Cleavage in Agrobacterium. PNAS 85, 8558-8562.
Nicot, N., Hausman, J.-F., Hoffmann, L., & Evers, D. (2005). Housekeeping gene selection for real-time R T-PCR normalization in potato during biotic and abiotic stress. Journal of Experimental Botany, 56(421), 2907-14.
Gray, G. S., and Fitch, W. M. (1983). Evolution of Antibiotic Resistance Genes: The DNA Sequence of a Kanamycin Resistance Gene from Staphylococcus Aureus. Molecular Biology and Evolution 1,57-66.
Jimenez, A. and Davies, J. (1980) Expression of a transposable antibiotic resistance element in Saccharomyces, Nature, 287,869-871.
Davies, J. and Smith, D. I. (1978) Plasmid-determined resistance to antimicrobial agents. Annu. Rev. Microbiol., 32,469-518.
Gutierrez Sanchez, P. A., Babujee, L., Jaramillo Mesa, H. et al. Overexpression of a modified eIF4E regulates potato virus Y resistance at the transcriptional level in potato. BMC Genomics 21,18 (2020).
Grech-Baran M, Witek K, Szajko K, Witek A I, Morgiewicz K, Wasilewicz-Flis I, Jakuczun H, Marczewski W, Jones JDG, Hennig J. Extreme resistance to Potato virus Y in potato carrying the Ryₛₜₒ gene is mediated by a TIR-NLR immune receptor. Plant Biotechnol J. 2020 March; 18(3):655-667.
Cavatorta J, Perez K W, Gray S M, Van Eck J, Yeam I, Jahn M. Engineering virus resistance using a modified potato gene. Plant Biotechnol J. 2011 December; 9(9):1014-21.
Kaniewski, W. K., and Thomas, P. E., 2004. The potato story. AgBioForum 7: 41-46.
Song Y, Zhang Z, Seidl M F, Majer A, Jakse J, Javornik B, Thomma B P. Broad taxonomic characterization of Verticillium wilt resistance genes reveals an ancient origin of the tomato Vel immune receptor. Mol Plant Pathol. 2017 February; 18(2):195-209
Jun-Zhi Wei, Kristina Hale, Lynn Carta, Edward Platzer, Cynthie Wong, Su-Chiung Fang, Raffi V. Aroian, Bacillus thuringiensis crystal proteins that target nematodes Proceedings of the National Academy of Sciences (PNAS March 2003, 100 (5) 2760-2765
Xiang-Qian Li, Anderson Tan, Michael Voegtline, Senait Bekele, Chang-Shi Chen, Raffi V. Aroian, Expression of Cry5B protein from Bacillus thuringiensis in plant roots confers resistance to root-knot nematode, Biological Control, Volume 47, Issue 1, October 2008, Pages 97-102
Du and Vleeshouwers (2017) New Strategies Towards Durable Late Blight Resistance in Potato in Pages 161-169 of Book titled “The Potato Genome”, Springer;
Witek et al. (2021) A complex resistance locus in Solanum americanum recognizes a conserved Phytophthora effector. Nature Plants 7: 198-208
Zhu et al. (2012) Functional stacking of three resistance genes against Phytophthora infestans in potato. Transgenic Res. 21(1):89-99

### NUMBERED EMBODIMENTS OF THE DISCLOSURE

Notwithstanding the appended claims, the disclosure sets forth the following numbered embodiments:

1. A transfer-DNA (T-DNA) characterized by a plurality of expression cassettes, comprising:

at least one resistance (R) gene to potato late blight;

a polynucleotide for decreasing acrylamide;

a polynucleotide for reducing black spot; and

a polynucleotide conferring protection to potato virus Y,

wherein each expression cassette comprises at least one promoter.

2. The T-DNA of embodiment 1, wherein the T-DNA comprises two resistance (R) genes to potato late blight.

3. The T-DNA of embodiment 1, wherein the T-DNA comprises three resistance (R) genes to potato late blight.

4. The T-DNA of any one of embodiments 1-3, wherein the resistance (R) genes are selected from the group consisting of Rpi-Vnt1, Rpi-Amr3, Rpi-Blb2, RpiBlb1, Rpi-mcq1, Rpi-Amr1, and Rpi-chc1.

5. The T-DNA of any one of embodiments 1-4, wherein the polynucleotide for decreasing acrylamide is a silencing element selected from the group consisting of: an inverted repeat of a vacuolar invertase (VInv) gene fragment, an inverted repeat of asparagine synthetase-1 gene (Asn1) fragment, an inverted repeat of asparagine synthetase-2 gene (Asn2) fragment, an inverted repeat of phosphorylase-L (PhL) fragment, and an inverted repeat of water dikinase-(R1) gene fragment.

6. The T-DNA of any one of embodiments 1-4, wherein the polynucleotide for reducing black spot is a silencing element comprising an inverted repeat of a polyphenol oxidase gene (Ppo) fragment.

7. The T-DNA of any one of embodiments 1-4, wherein the polynucleotide conferring protection to potato virus Y is selected from the group consisting of: a silencing element comprising an inverted repeat of a potato virus Y coat protein-encoding gene (PVY-Cp) fragment, overexpression of eukaryotic translation initiation factor (eIF4E) gene, and overexpression of a Rysto gene derived from Solanum stoloniferum.

8. The T-DNA of any one of embodiments 1-7, wherein the polynucleotide for decreasing acrylamide lowers the level of reducing sugars, which confers resistance to cold-induced sweetening.

9. The T-DNA of embodiment 1, wherein the plurality of expression cassettes comprises five cassettes, comprising:

a Rpi-Vnt1 resistance (R) gene;

a Rpi-Amr3 resistance (R) gene;

a Rpi-Blb2 resistance (R) gene;

an inverted repeat of vacuolar invertase (VInv) gene fragment and an inverted repeat of polyphenol oxidase (Ppo) gene fragment; and

an inverted repeat of a potato virus Y coat protein-encoding gene (PVY-Cp) fragment.

10. The T-DNA of embodiment 1, wherein the plurality of expression cassettes comprises six cassettes, comprising:

a Rpi-Vnt1 resistance (R) gene;

a Rpi-Amr3 resistance (R) gene;

a Rpi-Blb2 resistance (R) gene;

an inverted repeat of vacuolar invertase (VInv) gene fragment;

an inverted repeat of polyphenol oxidase (Ppo) gene fragment; and

an inverted repeat of a potato virus Y coat protein-encoding gene (PVY-Cp) fragment.

11. The T-DNA of embodiment 9 or 10, further comprising a Rpi-Vnt1 promoter and terminator operably linked to the Rpi-Vnt1 gene.

12. The T-DNA of embodiment 9 or 10, further comprising a Rpi-Amr3 promoter and terminator operably linked to the Rpi-Amr3 gene.

13. The T-DNA of embodiment 9 or 10, further comprising a Rpi-Blb2 promoter and terminator operably linked to the Rpi-Blb2 gene.

14. The T-DNA of embodiment 9 or 10, further comprising a granule-bound starch synthase gene (pGbss) promoter and an ADP glucose pyrophosphorylase gene (pAgp) promoter operably linked to the inverted repeat of VInv gene fragment.

15. The T-DNA of embodiment 9 or 10, further comprising a granule-bound starch synthase gene (pGbss) promoter and an ADP glucose pyrophosphorylase gene (pAgp) promoter operably linked to the inverted repeat of Ppo gene fragment.

16. The T-DNA of embodiment 9 or 10, further comprising a polyubiquitin promoter (pUbi7) and terminator (tUbi3) operably linked to the PVY-Cp gene fragment.

17. The T-DNA of any one of embodiments 1-16, further comprising an expression cassette comprising a selection marker, a polynucleotide conferring protection to Potato virus X, a polynucleotide conferring protection to Potato leaf roll virus, a polynucleotide conferring protection to tobacco rattle virus, a polynucleotide conferring protection to verticillium wilt, and/or a polynucleotide conferring protection to a parasitic nematode.

18. The T-DNA of embodiment 17, wherein the selection marker is a modified acetolactate synthase gene (StmAls).

19. The T-DNA of embodiment 18, further comprising a polyubiquitin promoter (pUbi7) and terminator (tUbi3) operably linked to the StmAls gene.

20. The T-DNA of embodiment 18 or 19, wherein a polypeptide encoding the modified ALS protein comprises the at least one modification at an amino acid residue corresponding to position W563, or position 5642, or combination thereof.

21. The T-DNA of embodiment 20, wherein the at least one modification at the amino acid residue corresponding to position W563 is Leu (L).

22. The T-DNA of embodiment 20, wherein the at least one modification at the amino acid residue corresponding to position S642 is Ile (I).

23. A T-DNA region comprising the nucleotide sequence set forth in SEQ ID NO:2 or a sequence 90% identical thereto.

24. A T-DNA region comprising the nucleotide sequence set forth in SEQ ID NO:3 or a sequence 90% identical thereto.

25. The T-DNA of any one of embodiments 1-24, wherein the T-DNA is inserted into a binary vector.

26. A potato transformation vector designated pSIM4363, wherein the vector includes a T-DNA region comprising the nucleotide sequence set forth in SEQ ID NO:2, and wherein the plant transformation vector was deposited under ATCC Accession No. PTA-126737.

27. A potato transformation vector designated pSIM4617, wherein the vector includes a T-DNA region comprising the nucleotide sequence set forth in SEQ ID NO:3, and wherein the plant transformation vector was deposited under ATCC Accession No. PTA-126738.

28. A potato plant, a part thereof, or a cell thereof, comprising the T-DNA of any one of embodiments 1-27, wherein the plant exhibits:

resistance to potato late blight caused by Phytophthora infestans;

increased black spot bruise tolerance;

decreased acrylamide; and

resistance to potato virus Y.

29. A tissue culture of cells produced from the plant of embodiment 28, wherein the tissue cultured cells comprise the T-DNA.

30. A potato plant, or a part thereof, regenerated from the tissue culture of embodiment 29, wherein the plant exhibits:

resistance to potato late blight caused by Phytophthora infestans;

increased black spot bruise tolerance;

decreased acrylamide; and

resistance to potato virus Y.

31. A potato seed produced by growing the potato plant of embodiment 28 or 30, wherein the seed comprises the T-DNA.

32. A potato plant, or a part thereof, produced by growing the seed of embodiment 31.

33. A commodity plant product produced from the potato plant of any one of embodiments 28, 30, or 32, wherein the product comprises the T-DNA.

34. A potato plant, tuber, or a part thereof, comprising the T-DNA insert region of the plant transformation vector pSIM4363, wherein vector pSMIM4363 was deposited under ATCC Accession No. PTA-126737.

35. A potato plant, tuber, or a part of a tuber, comprising the T-DNA insert region of the plant transformation vector pSIM4617, wherein vector pSIM4617 was deposited under ATCC Accession No. PTA-126738.

36. A food product made from the potato tuber of embodiment 34 or 35.

37. A food product made from the potato tuber of embodiment 34 or 35, wherein the food product is a sliced potato tuber food product.

38. A food product made from the potato tuber of embodiment 34 or 35, wherein the food product is a French fry or chip.

39. A heat-processed tuber product obtained from the potato tuber of embodiment 34 or 35.

40. A heat-processed tuber product obtained from the potato tuber of embodiment 34 or 35, wherein the heat processed tuber product is selected from the group consisting of a French fry, a chip, and a baked potato.

41. A method for detecting presence of a target insert region in a nucleic acid sample, the method comprising:

(i isolating the nucleic acid sample from a plant, or plant part, or plant-derived food product;

(ii) screening presence of the target insert region of pSIM4363 or pSIM4617, and

(iii) selecting a transformed plant with the target insert region.

42. The method of embodiment 41, wherein the presence of the target insert region is determined by polymerase-chain reaction (PCR) with a forward primer and a reverse primer.

43. The method of embodiment 41, wherein the PCR is standard PCR, quantitative PCR, PCR arrays, chip PCR, reverse-transcriptase PCT, multiplext PCR, nested PCR, long-range PCR or GC-rich PCR.

44. The method of embodiment 41, wherein the forward primer sequence comprises SEQ ID NO:4, 36, 39, 42, 45, 48, 51, 54, 57, 60, 63, 66, 69, 72, 75, 78, 81, 84, 87, 90, 93, 96, or 99.

45. The method of embodiment 41, wherein the reverse primer sequence comprises SEQ ID NO:5, 37, 40, 43, 46, 49, 52, 55, 58, 61, 64, 67, 70, 73, 76, 79, 82, 85, 88, 91, 94, 97, or 100.

46. The method of embodiment 41, wherein the plant is a potato plant.

47. The method of embodiment 41, wherein the plant part is selected from the group consisting of potato flowers, potato tepals, potato petals, potato sepals, potato anthers, potato pollen, potato seeds, potato leaves, potato petioles, potato stems, potato roots, potato rhizomes, potato stolons, potato tubers, potato shoots, potato cells, potato protoplasts, potato plant tissues, and combinations thereof

48. The method of embodiment 41, wherein the plant-derived food product is selected from the group consisting of a potato processed food product, a potato livestock feed material, French fries, potato chips, dehydrated potato material, potato flakes, potato granules, potato protein powder, potato starch, potato flour, instant potato products, and combinations thereof.

1. A transfer-DNA (T-DNA) characterized by a plurality of expression cassettes, comprising:
at least one resistance (R) gene to potato late blight;
a polynucleotide for decreasing acrylamide;
a polynucleotide for reducing black spot; and
a polynucleotide conferring protection to potato virus Y,
wherein each expression cassette comprises at least one promoter.

2. The T-DNA of claim 1, wherein the T-DNA comprises two resistance (R) genes to potato late blight.

3. The T-DNA of claim 1, wherein the T-DNA comprises three resistance (R) genes to potato late blight.

4. The T-DNA of claim 1, wherein the resistance (R) genes are selected from the group consisting of Rpi-Vnt1, Rpi-Amr3, Rpi-B1b2, RpiBlb1, Rpi-mcq1, Rpi-Amr1, and Rpi-chc1.

5. The T-DNA of claim 1, wherein the polynucleotide for decreasing acrylamide is a silencing element selected from the group consisting of: an inverted repeat of a vacuolar invertase (VInv) gene fragment, an inverted repeat of asparagine synthetase-1 gene (Asn1) fragment, an inverted repeat of asparagine synthetase-2 gene (Asn2) fragment, an inverted repeat of phosphorylase-L (PhL) fragment, and an inverted repeat of water dikinase-(R1) gene fragment.

6. The T-DNA of claim 1, wherein the polynucleotide for reducing black spot is a silencing element comprising an inverted repeat of a polyphenol oxidase gene (Ppo) fragment.

7. The T-DNA of claim 1, wherein the polynucleotide conferring protection to potato virus Y is selected from the group consisting of: a silencing element comprising an inverted repeat of a potato virus Y coat protein-encoding gene (PVY-Cp) fragment, overexpression of eukaryotic translation initiation factor (eIF4E) gene, and overexpression of a Rysto gene derived from Solanum stoloniferum.

8. The T-DNA of claim 1, wherein the polynucleotide for decreasing acrylamide lowers the level of reducing sugars, which confers resistance to cold-induced sweetening.

9. The T-DNA of claim 1, wherein the plurality of expression cassettes comprises five cassettes, comprising:
a Rpi-Vnt1 resistance (R) gene;
a Rpi-Amr3 resistance (R) gene;
a Rpi-Blb2 resistance (R) gene;
an inverted repeat of vacuolar invertase (VInv) gene fragment and an inverted repeat of polyphenol oxidase (Ppo) gene fragment; and
an inverted repeat of a potato virus Y coat protein-encoding gene (PVY-Cp) fragment.

10. The T-DNA of claim 1, wherein the plurality of expression cassettes comprises six cassettes, comprising:
a Rpi-Vnt1 resistance (R) gene;
a Rpi-Amr3 resistance (R) gene;
a Rpi-Blb2 resistance (R) gene;
an inverted repeat of vacuolar invertase (VInv) gene fragment;
an inverted repeat of polyphenol oxidase (Ppo) gene fragment; and
an inverted repeat of a potato virus Y coat protein-encoding gene (PVY-Cp) fragment.

11. The T-DNA of claim 9, further comprising a Rpi-Vnt1 promoter and terminator operably linked to the Rpi-Vnt1 gene.

12. The T-DNA of claim 9, further comprising a Rpi-Amr3 promoter and terminator operably linked to the Rpi-Amr3 gene.

13. The T-DNA of claim 9, further comprising a Rpi-Blb2 promoter and terminator operably linked to the Rpi-Blb2 gene.

14. The T-DNA of claim 9, further comprising a granule-bound starch synthase gene (pGbss) promoter and an ADP glucose pyrophosphorylase gene (pAgp) promoter operably linked to the inverted repeat of VInv gene fragment.

15. The T-DNA of claim 9, further comprising a granule-bound starch synthase gene (pGbss) promoter and an ADP glucose pyrophosphorylase gene (pAgp) promoter operably linked to the inverted repeat of Ppo gene fragment.

16. The T-DNA of claim 9, further comprising a polyubiquitin promoter (pUbi7) and terminator (tUbi3) operably linked to the PVY-Cp gene fragment.

17. The T-DNA of claim 1, further comprising an expression cassette comprising a selection marker, a polynucleotide conferring protection to Potato virus X, a polynucleotide conferring protection to Potato leaf roll virus, a polynucleotide conferring protection to tobacco rattle virus, a polynucleotide conferring protection to verticillium wilt, and/or a polynucleotide conferring protection to a parasitic nematode.

18. The T-DNA of claim 17, wherein the selection marker is a modified acetolactate synthase gene (StmAls).

19. The T-DNA of claim 18, further comprising a polyubiquitin promoter (pUbi7) and terminator (tUbi3) operably linked to the StmAls gene.

20. The T-DNA of claim 18, wherein a polypeptide encoding the modified ALS protein comprises the at least one modification at an amino acid residue corresponding to position W563, or position 5642, or combination thereof

21. The T-DNA of claim 20, wherein the at least one modification at the amino acid residue corresponding to position W563 is Leu (L).

22. The T-DNA of claim 20, wherein the at least one modification at the amino acid residue corresponding to position S642 is Ile (I).

23. The T-DNA of claim 1, wherein the T-DNA comprises a nucleotide sequence set forth in SEQ ID NO:2 or a sequence 90% identical thereto.

24. The T-DNA of claim 1, wherein the T-DNA comprises a nucleotide sequence set forth in SEQ ID NO:3 or a sequence 90% identical thereto.

25. The T-DNA of claim 1, wherein the T-DNA is inserted into a binary vector.

26. A potato transformation vector designated pSIM4363, wherein the vector includes a T-DNA region comprising the nucleotide sequence set forth in SEQ ID NO:2, and wherein the plant transformation vector was deposited under ATCC Accession No. PTA-126737.

27. A potato transformation vector designated pSIM4617, wherein the vector includes a T-DNA region comprising the nucleotide sequence set forth in SEQ ID NO:3, and wherein the plant transformation vector was deposited under ATCC Accession No. PTA-126738.

28. A potato plant, a part thereof, or a cell thereof, comprising the T-DNA of claim 1, wherein the plant exhibits:
resistance to potato late blight caused by Phytophthora infestans;
increased black spot bruise tolerance;
decreased acrylamide; and
resistance to potato virus Y.

29. A tissue culture of cells produced from the plant of claim 28, wherein the tissue cultured cells comprise the T-DNA.

30. A potato plant, or a part thereof, regenerated from the tissue culture of claim 29, wherein the plant exhibits:
resistance to potato late blight caused by Phytophthora infestans;
increased black spot bruise tolerance;
decreased acrylamide; and
resistance to potato virus Y.

31. A potato seed produced by growing the potato plant of claim 28, wherein the seed comprises the T-DNA.

32. A potato plant, or a part thereof, produced by growing the seed of claim 31.

33. A commodity plant product produced from the potato plant of claim 28, wherein the product comprises the T-DNA.

34.-48. (canceled)
